(19) United States
(12) Patent Application Publication
(10) Pub. No.: US 2021/0023223 A1
(43) Pub. Date: Jan. 28, 2021

(54) HYDROCARBON / LIPID - CAROTENOID COMPLEXES

(71) Applicant: IP SCIENCE LIMITED, Cambridge, Cambridgeshire (GB)

(72) Inventor: Ivan PETYAEV, Cambridge, Cambridgeshire (GB)

(73) Assignee: IP SCIENCE LIMITED, Cambridge, Cambridgeshire (GB)

(21) Appl. No.: 16/967,843

(22) PCT Filed: Feb. 7, 2019
(86) PCT No.: PCT/GB2019/050333

§ 371 Date: Aug. 6, 2020

(30) Foreign Application Priority Data
Feb. 7, 2018 (GB) 1801985.1

(51) Int. Cl. A61K 47/06; A61K 31/202; A23D 9/007; A23G 9/32; A61K 9/10; A61K 9/107; A23L 33/115; A23P 10/35; A23L 25/10; A61K 8/04; A23C 9/13; A61Q 19/06; A61K 31/122; A23G 1/36; A61K 8/31; C10L 1/185; A61K 31/685; A61Q 19/00; A23L 5/44; A23C 15/12; A23C 9/152; A61K 8/35; A61K 9/06; C10L 1/16; A61K 31/01; A61K 8/06

(52) U.S. Cl. CPC A61K 47/06; C10L 2270/08; A23D 9/007; A23G 9/327; A61K 9/10; A61K 9/107; A23L 33/115; A23P 10/35; A23L 25/10; A61K 8/04; A23C 9/1307; A61Q 19/06; A61K 31/122; A23G 1/36; A61K 8/31; C10L 1/1857; A61K 31/685; A61Q 19/00; A23L 5/44; A23C 15/12; A23C 9/1528; A61K 8/35; A61K 9/06; C10L 1/1608; A61K 31/01; A61K 8/06; A23V 2002/00; C10L 2230/22; A61K 31/202

(57) ABSTRACT

The invention relates to uses of hydrocarbon/lipid complexes with carotenoids for improvement properties of hydrocarbon-based products as well as related methods and uses.

### INTRODUCTION

Modification and diversification of physical and chemical properties of long-chain hydrocarbons, for example lipids, is one of the essential objectives in the history of technology evolution, whether it is in the field of foods, health, fuels, electronics or engineering. There are a number of processes and/or other types of agents or molecules that are used for these purposes. Hydrocarbons are present in a number of industrially important compounds, including foodstuff and fuel. Modifying the properties of hydrocarbons within such products, for example reducing viscosity, thermal energy storage and conductivity or altering other physical characteristics can have a wide variety of applications. The present invention is aimed at addressing these areas.

### SUMMARY

The inventor has surprisingly found that carotenoids can form physical complexes with hydrocarbons, which significantly changes the properties of hydrocarbons. As illustrated in the examples, when carotenoids were incorporated into hydrocarbons, in particular lipid structures, this resulted in a significant change of hydrocarbon architecture as well as a change in physical and other properties. Without wishing to be bound by theory, we believe that these changes cannot be attributed to mechanical interactions of carotenoids with lipids and other hydrocarbons, but to a new phenomenon of disruption of their configuration, such as folding, structural organisation, packing and interaction of the hydrocarbon molecules with each other.

This invention relates to hydrocarbon particles, complexes or continuum matrixes which have embedded in their structure a carotenoid, methods for making such hydrocarbons, use of a carotenoid in changing/disrupting the configuration, structural organisation, for example folding, packing and interaction of a hydrocarbon molecules and to methods for measuring the disruption of the configuration, structural organisation, packing and interaction of hydrophobic molecules by carotenoids. The invention also relates to a formulation comprising such particles or complexes.

The disruption of the structure of a hydrocarbon results in a change of their physical and chemical properties, which can be exploited as set out in the detailed description.

One or more of the following properties may be changed: viscosity, melting point, thermal energy storage and conductivity, resistance to oxidation and degradation or digestibility.

### FIGURES

The invention is further illustrated in the following non-limiting figures.

FIG. 1. Lutein: Meso-Zeaxanthin, 50:50, increases lipid size droplets of dairy butter

FIG. 2. Increase of size of pork fat globules by lycopene

FIG. 3. Increase of the size of beef fat globules by lycopene.

FIG. 4 A-C. A—Increase of the size of beef fat globules by astaxanthin. B—Control effect of lycopene on the size of cocoa butter fat globules; C—lycopene in cocoa butter ratio 1:40,000.

FIG. 5. Reduction of the dairy butter viscosity by astaxanthin.

FIG. 6 A-D. A—Lycopene reduces lipid viscosity assessed by the size of the lipid drops on the water surface. Labels: from left to right for each chart: control 0.5 mg/30 ml, 1.0 mg/30 ml, 3.5 mg/30 ml, 7.0 mg/30 ml, 10.0 mg/30 ml. B—Control cocoa butter; C—Lycopene increases surface size of a molten Cocoa butter drop; the drop mass was in both cases 23±0.15 mg. D-Lycopene reduces Doxosahexaenoic Acid, DHA, viscosity and enlarges lipid surface at pH 2.5; left—control DHA, right with 0.5% lycopene; drop mass in both cases 24±0.2 mg

FIG. 7. Oil volume (ml) required for achieving unbroken oil overlay formation on the surface of a 100 ml water sample.

FIG. 8. Reduction of melting time of dairy butter by astaxanthin.

FIG. 9. Reduction of melting time of pork fat by lycopene.

FIG. 10. Reduction of melting time of pork fat by astaxanthin. Labels: from left to right for each chart: control, lycopene, lutein, astaxanthin.

FIG. 11. Reduction of melting time of beef fat by lycopene.

FIG. 12. Reduction of melting time of beef fat by astaxanthin.

FIG. 13. Reduction of melting time of cocoa butter by lycopene.

FIG. 14. Reduction of melting time of white chocolate butter by lycopene.

FIG. 15. Reduction of melting time of dark chocolate by different carotenoids.

FIG. 16. Reduction of melting time of dark chocolate by Lutein in different concentrations

FIG. 17. Reduction of melting time of dark chocolate by Zeaxanthin and Lutein-Zeaxanthin at different concentrations. Labels: from left to right for each chart: melting times for zeaxanthin chocolate, melting times for zeaxanthin+lutein chocolate.

FIG. 18. Reduction of melting time of chocolate spread by lycopene.

FIG. 19. Reduction of melting time of peanut butter by lycopene.

FIG. 20 A-B. Astaxanthin creates a physical complex with cocoa butter which affects its viscosity and helps to reduce chocolate bloom; Green & Black's Cocoa 70%+15% hazelnuts—100 g; A) control; B) chocolate with 40 mg astaxanthin.

FIG. 21. Reduction of melting time of frozen olive oil by lycopene.

FIG. 22. Astaxanthin reduces melting time of frozen olive oil-dose dependent effect. Astaxanthin concentration per 30 grams of the oil.

FIG. 23. β-Carotene reduces melting time of frozen olive oil-dose dependent effect, concentration per 30 grams of the oil.

FIG. 24. Astaxanthin reduces melting time of frozen sunflower oil-dose dependent effect. Astaxanthin concentration per 30 grams of the oil.

FIG. 25. Reduction of melting time of frozen sunflower oil by lycopene.

FIG. 26. Reduction of melting time of frozen sunflower oil by β-Carotene.

FIG. 27. Reduction of melting time of frozen canola oil by lycopene.

FIG. 28. Reduction of melting time of frozen cod liver oil by lycopene.

FIG. 29. Reduction of melting time of frozen cod liver oil by astaxanthin

FIG. 30 A-C. A)—Reduction of melting time of lamp oil by astaxanthin and lycopene. B) Control palm oil; C) Anti-freezing effect of lycopene on solidifying Palm Oil. 24 h after a drop of molten at 40° C. oil, with or without lycopene, was place on water surface at 20° C.; oil drop mass in both cases was 23±0.1 mg.

FIG. 31. Lycopene accelerate bringing dairy butter to its boiling point.

FIG. 32. Carotenoids accelerate olive oil-in-water 50:50 emulsion heating.

FIG. 33. Carotenoids accelerate heating of water—olive oil mixture in ratio: 75%:25%.

FIG. 34. An explosion of a hydrocarbon—lycopene complex after one thermo-cycle: from +20° C. to −18° C. for 24 hours, and brining back to +20° C.

FIG. 35. Acceleration of cooking time of chicken liver by olive oil with carotenoids.

FIG. 36. Acceleration of cooking time of salmon fillet by olive oil with carotenoids.

FIG. 37. Acceleration of cooking time of tuna fillet by olive oil with astaxanthin.

FIG. 38. Effect of carotenoids on cooking time of marinated lamb steak.

FIG. 39. Dynamics of internal temperature changes during cooking of chicken livers at 180° C. and addition of different ingredients (Astaxanthin, Lycopene, Olive Oil and Water respectively). Dotted lines of the same colour show samples cooked when fresh lemon juice was added. Results from the two cooking experiments were combined to prepare this graph (average temperatures are shown).

FIG. 40. Absolute values (results for undiluted samples) of Vitamin B12 concentrations in raw chicken liver (Fresh) and chicken liver cooked at 180° C. at different conditions (using olive oil containing 7 mg/ml of Lycopene, olive oil containing 7 mg/ml of Astaxanthin, pure olive oil and water respectively). Second bars presented for all variants of cooking show Vitamin B12 concentrations for chicken livers prepared in the same conditions, but with fresh lemon juice added.

FIG. 41. Dynamics of internal temperature changes during cooking of wild salmon pieces at 180° C. and addition of different ingredients (Astaxanthin, Lycopene, Olive Oil and Water respectively).

FIG. 42. Vitamin B12 concentrations in wild salmon cooked at 180° C. in different conditions (using olive oil containing 7 mg/ml of Astaxanthin, olive oil containing 7 mg/ml of Lycopene, pure olive oil and water respectively).

FIG. 43. Vitamin D3 concentrations in wild salmon cooked at 180° C. in different conditions (using olive oil containing 7 mg/30 ml of Astaxanthin, olive oil containing 7 mg/30 ml of Lycopene, pure olive oil and water respectively).

FIG. 44. Proportion of Vitamin B12 and Vitamin D3 retention (yellow/pale grey) and loss (black) in wild salmon cooked using olive oil supplemented with Lycopene (Lycopene), olive oil supplemented with Astaxanthin (Astaxanthin), pure olive oil (Olive oil) and without oil (Water).

FIG. 45 A-B. Lycopene changes in lipid folding of Cocoa butter reduces its digestibility by pancreatic lipase; 24 hours in PBS at 37° C., cocoa particle mass in both cases below was 22±0.11 mg. A—start of the experiment; B— after 24 hours.

FIG. 46. Lycopene stimulate formation of lipid droplets in B10.MLM cells.

The cells were set up and grown as described in the Material and Methods. Lipid droplets were visualized as described above in 24, 30 and 48 hours after lycopene addition. 1st column—control cells, 2nd column —cells incubated with olive oil only, 3rd column—cells incubated with oil-formulated lycopene, 4th column—cells incubated with starch only, 5th column—cells incubated with starch microencapsulated lycopene FIG. 47 A-B. A) Lycopene stimulate formation of lipid droplets within alveolar macrophages. Incubation time 48 hours, (↓) lipid droplets, lycopene concentration 0.5 mg/ml, ×6000; B—Lycopene stimulate formation of lipid droplets within alveolar macrophages. Incubation time 48 hours, (↓) lipid droplets, lycopene concentration 0.5 mg/ml, ×15,000.

FIG. 48 A-B. Lycopene stimulates formation of lipid droplets and growth of mitochondria in alveolar macrophages. A—oil form of lycopene and ascorbic acid after 48 hours incubation at ×17,000; B—microencapsulated lycopene and ascorbic acid following 48 hour incubation at ×17,000.

FIG. 49. Changes in the size of the lipid droplets collected from the surface of the skin of volunteers after 4 weeks of supplementation by 4 mg of formulated astaxanthin.

FIG. 50 A-B. A—Changes in the number of desquamated corneocytes and B—intensity of the bacteria load on the surface of the skin of volunteers after 4 weeks of supplementation by 4 mg of formulated astaxanthin.

FIG. 51 A-B. Typical example of changes in the number and the quality of desquamated corneocytes of the skin of a volunteer after 4 weeks of supplementation by 4 mg of formulated astaxanthin. A—before supplementation; B— after supplementation with 4 mg of formulated astaxanthin.

FIG. 52 A-B. A—Lycopene accumulation in sebum and B— desquamated corneocytes of the skin of a volunteer during 4 weeks of supplementation with 7 mg of formulated lycopene.

FIG. 53 A-B. Changes in the size of the lipid droplets collected from the surface of the skin of a middle-aged volunteer after 4 weeks of supplementation with 7 mg of formulated lycopene. A—at the start; B—after four weeks of supplementation with 7 mg of formulated lycopene.

FIG. 54 A-B. Changes in the gram-positive bacteria load of the skin of a middle-aged volunteer after 4 weeks of supplementation with 7 mg of formulated lycopene. A—before supplementation; B—after 4 weeks of supplementation with 7 mg of formulated lycopene.

FIG. 55 A-B. Changes in the numbers and quality of desquamated corneocytes size of the skin of a middle-aged volunteer after 4 weeks of supplementation with 7 mg of formulated lycopene. A—before supplementation; B—after 4 weeks of supplementation with 7 mg of formulated lycopene.

FIG. 56 A-B. Changes in the size of the lipid droplets collected from the surface of the skin of volunteers after 4 weeks of supplementation with a combination of 7 mg formulated Lutein and 1.4 mg Zeaxanthin. A before supplementation; B—after 4 weeks of supplementation with a combination of 7 mg formulated Lutein and 1.4 mg Zeaxanthin.

FIG. 57 A-B. Boost of sebum production by lycopene supplementation. A—before; B—after 4 weeks.

FIG. 58 A-B. Reduction of the exfoliation of corneocyte from the surface of the skin, and disappearance of their damaged clustered forms after 4 weeks of lycopene supplementation. A—before; B—after 4 weeks of lycopene supplementation.

FIG. 59 A-C. Therapeutic effect of lycopene supplementation on micro-abscess on the face of the patient—disappearance of gram-positive bacteria and inflammatory cells. A—before supplementation; B—after 2 weeks; C—after 3 weeks of supplementation.

FIG. 60. Red-shift in light absorption peaks of lycopene embedded into sunflower oil—blue, control lycopene—red, control sunflower oil—green.

FIG. 61. Red-shift in light absorption peaks of lutein embedded into sunflower oil—blue, control lycopene—red, control sunflower oil—green.

FIG. 62. Hyperchromism in light absorption peaks of lutein embedded into cocoa butter—blue, control lycopene—red, control cocoa butter—green.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

This invention utilises a hitherto unknown property of carotenoids, which is based on their ability to create physical complexes with other hydrocarbons, and in particular with long-chain hydrocarbons, for example lipids, or other hydrophobic molecules, which can disrupt their configuration, structural organisation, packing and interaction with each other. This results in products with new beneficial physical and chemical parameters, which can be used in a range of different fields and applications. For example, these applications include, but are not limited to the following, which are all within the scope of the invention.

Food Engineering

In the field of food engineering, embedding carotenoids in a hydrocarbon, thereby changing the folding structure of the hydrocarbon, can result in one or more of the following:
1) disruption of lipid folding which results in a change of physical and consumer properties not only for fat or oil based products, but also for products where lipids are only a part of the food matrix, or of a beverage volume, but still keep their new properties, which would benefit the whole product or beverage properties;
2) increase of spreadability of oil, butter, margarine and other fat-based, or fat-rich, products;
3) reduction of susceptibility of a hydrocarbon/lipid containing foodstuff, pharmaceutical, nutraceutical or other bioactive molecules blended/incorporated into this hydrocarbon, for example lipids, to acidic, or other forms of oxidation or degradation in product matrixes;
4) reduction of a digestibility rate of a hydrocarbon/lipid containing foodstuff, pharmaceutical, nutraceutical or other bioactive molecules blended/incorporated into this hydrocarbon, for example lipids, by enzymatic catalysis;
5) reduction of melting and defrosting time or anti-freezing of lipid containing food stuff;
6) reduction or prevention of blooming of chocolate and improve its taste,
7) improvement of the taste of ice cream and other fat containing food or beverages;
8) improvement of cooking oils and fats by increasing the rate of heating of the cooking oils and fats, resulting in reduced cooking time, helping to create healthier food/meal containing more heat-sensitive nutrient, micro-nutrients and vitamins;

Nutrition, Pharmaceuticals and Cosmetics

In the field of nutrition, pharmaceuticals and cosmetics, embedding carotenoids in a hydrocarbon, thereby changing the folding structure of the hydrocarbon can result in:
1) increasing the surface of droplets, continuum or other hydrocarbon/lipid formations, including pharmaceutical, nutraceutical or other bioactive molecules blended/incorporated into this hydrocarbon, for example lipids, which reduces their ability of oxidise in general, and acid modification or degradation in particular, for example, but not limited in the stomach environment, which may result in improvement of the absorption of these molecules in unmodified forms, hence their bioavailability and clinical efficacy;
2) creating nutritional products with increased size of lipid droplets or globules. This results in reduced viscosity and accelerated gastro-intestinal transit time, with lowered rate of lipid digestion, and consequently reduced postprandial lipidaemia and absorption of calories-rich lipids. This can help to improve weight management, control lipid metabolism, metabolic syndrome and obesity;
3) nutritional, nutraceutical or pharmaceutical products to activate intracellular lipid droplet formation:
   a) which, on the one hand, can boost mitochondrial respiration which is essential in stimulation of not-shivering thermogenesis and activation of beige and brown fat metabolism,
   b) and, on the one hand, can stimulate physical and mental performance, boost oxygen metabolism in sub-clinical or clinical hypoxia, and in regenerating, ageing and cancerous tissues, etc.;
4) nutritional, nutraceutical or pharmaceutical products to increase size of circulating plasma lipoproteins, important new therapeutic approach to prevent and treat atherosclerosis. The larger the lipids, the less likely it is that atherosclerosis occurs. In other words, smaller lipids are known to be causing atherosclerosis. Thus, including carotenoids in a functional food as described herein can prevent atherosclerosis.
5) nutritional, nutraceutical or pharmaceutical products to boost hydrophobicity of circulating plasma lipoproteins, and hence their ability to transport molecular oxygen to the tissues;
6) nutritional, cosmetic, nutraceutical or pharmaceutical products to reduce the viscosity of the sebum and boost its declined production in ageing, stress or disease, this would improve the lubrication and nourishing of the skin tissue, reduce its dryness, reduce level of desquamation of corneocytes and the level of their damage, and improve control of skin microbiota, increase skin protection from the UV-light, pollution and other environmental damaging factors;
7) restoration and improvement of lipid-containing secretions and fluids enriched with carotenoids can increase presence of these molecules on the surface of the eyes, lining in the mouth, throat, nose, ear, trachea, bronchi, pulmonary alveoli, ureter, urinary bladder, urethra, fallopian tube, vagina, nose mucosae, joint cavity, synovial membrane. In particular, it is the lubrications of the linings of the various organs and part of the body as described above that can help to maintain the right environment for certain advantageous bacteria to grow, for example Micrococcus luteus on the surface of the skin. This can have a positive effect on the immune system and a healthy microbiome can improve conditions such as skin conditions, for example those associated with dryness.
8) cosmetic, nutraceutical or pharmaceutical products such as oils, ointments, lotions, waxes, suppositories, creams with reduced viscosity and reduced melting or cooling time.

Machinery, Fuel, Etc.

In the field of machinery, fuel, and the like, hydrocarbon complexes with embedded carotenoids, which change the configuration, structural organisation, packing and interaction of its molecules with each other, can result in one or more of:
1) reduction of the viscosity of lubricants;
2) reduction of the freezing point of antifreeze;
3) being used as an anti-freeze;
4) reduction of viscosity, melting and boiling points, improving combustion and efficiency of biodiesels or bio-fuels, or other hydrocarbon fuels;
5) increase of thermal energy storage,
6) increase of thermal energy conductivity,
7) accelerating heating and cooling of fuel,
8) increase of burning time and heat generation of fuel without increased or with reduced fuel consumption.

This invention thus relates to hydrocarbon particles which have embedded in their structure a carotenoid, that is particles where the hydrocarbon forms a complex with a carotenoid. The invention thus relates to hydrocarbon/carotenoid complexes. Thus, the caroteinoid/hydrocarbon complex is formed by an association between the hydrocarbon and carotenoid. The invention also relates to methods for making such hydrocarbons, use of a carotenoid in changing/disrupting the configuration, structural organisation, for example folding, packing and interaction of the molecules of a hydrocarbon, and methods for measuring of the disruption of the configuration, structural organisation, packing and interaction of the molecules of a hydrocarbon by carotenoids as detailed below.

In a first aspect, the invention relates to a complex comprising a hydrocarbon and a carotenoid wherein said carotenoid is embedded in the hydrocarbon.

In one embodiment, the ratio of carotenoid:hydrocarbon, e.g. lipid as described in the various aspects herein is 1:100 to 1:1,000,000.

Thus, the carotenoid is embedded in the hydrocarbon, that is, it is incorporated into the hydrocarbon structure, creating a physical complex. Thus, the carotenoid is not merely present as a mixture with the hydrocarbon. The carotenoid and the hydrocarbon, for example a lipid, thus form a physical complex. The physical and functional properties of the hydrocarbon are changed due to the embedded carotenoid and such properties, for example light absorption, can be measured as explained below.

A hydrocarbon can be selected from an industrial, mechanical, technical or cosmetic oil, fat, wax, lubricant, greases, antifreeze, bio- or other fuel, hydraulic and other engine and machinery liquid. A hydrocarbon can be part of a therapeutic, cosmetic or personal hygiene oil, ointment, cream, wax or suppository.

In one embodiment, the hydrocarbon is a long chain hydrocarbon. In one embodiment, the hydrocarbon is a lipid. The term lipid(s) as used herein includes hydrophobic or amphiphilic small molecules, in particular fatty acids and or their derivatives, fats or wax. Lipids can be chemically synthetised, industrially produced by bacteria or fungi, or assembled in vivo, in humans, or animals, or vertebra, or plants. A lipid can be selected from products comprising fatty acids, monoglycerides or diglycerides or triglycerides or other glycerolipids, phosphatic acid or phosphatidylethanolamine or phosphatidylcholine or phosphatidylserine or phosphatidylinositol or other glycerophospholipids, ceramides or sphingolipids, sterols, waxes, fat-soluble vitamins, prenols, saccharolipids, polyketides, or their derivatives in pure, or blended, or co-synthesised, or co-produced, or co-existing with each other from the above list, or with other molecules or substances, forms.

Preferred lipids are: fatty acids, glycerolipids, glycerophospholipids, sphingolipids, sterols, waxes.

Carotenoid compounds are tetraterpenoids, which contain long polyene chains. Carotenoid compounds include xanthophylls such as lutein and zeaxanthin, and carotenes, such as beta-carotene, alpha-carotene, zeto-carotene, and lycopene compounds.

In a particular embodiment, the carotenoid is a xanthophyll. In one embodiment, the xanthophyll is selected from the group consisting of α-cryptoxantin, β-cryptoxantin, adonirubin, adonixanthin, alloxanthin, amarouciaxanthin A, antheraxanthin, astaxanthin, auroxanthin, caloxanthin, cantaxanthin, capsanthin, capsanthin-5-6-epoxide, capsorubin, crocoxanthin, diadinoxanthin, diatoxanthin, echinenone, fucoxanthin, fucoxanthinol, iso-fucoxanthin, iso-fucoxanthinol, lutein, luteoxanthin, mutatoxanthin, neoxanthin, nostoxanthin, violaxanthin, zeaxanthin and combinations thereof.

In one embodiment, the carotenoid is a carotene. In another embodiment, the carotene is selected from the group consisting of α-carotene, β-carotene, γ-carotene, δ-carotene, ε-carotene, ζ-carotene, lycopene, neurosporene, phytoene, phytofluene and combinations thereof.

In one embodiment, the carotenes and xantophylles described above refer to the all-trans forms thereof. In another embodiment, the xantophylles and carotenes for use in the present invention include derivatives containing one or more cis double bond.

In one embodiment, the carotenoid compound is a lycopene compound. Lycopene compounds may include lycopene, I-HO-3′, 4′-didehydrolycopene, 3, 1′-(HO) 2-gamma-carotene,1, 1′-(HO) 2-3, 4, 3′, 4′-tetradehydrolycopene, 1, 1′-(HO) 2-3, 4-didehydrolycopene.

In some embodiments, the carotenoid compound is a lycopene compound such as lycopene. Lycopene is an open-chain unsaturated C40 carotenoid of structure I (Chemical Abstracts Service Registry Number 502-65-8, C₄₀H₅₆).

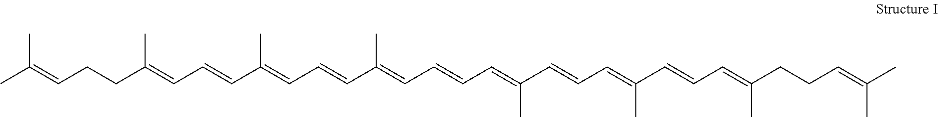 [SMILES: CC(C)=CCC/C(C)=C/C=C/C(C)=C/C=C/C(C)=C/C=C/C=C(C)/C=C/C=C(C)/C=C/C=C(\C)CCC=C(C)C]

Lycopene occurs naturally in plants such as tomatoes, guava rosehip, watermelon and pink grapefruit and any such sources of lycopene may be, for instance, employed.

Lycopene for use as described herein may comprise one or more different isomers. For example, lycopene may include cis-lycopene isomers, trans-lycopene isomers and mixtures of the cis- and trans-isomers. Lycopene may comprise at least 10%, at least 20%, at least 30%, at least 40%, at least 50%, at least 60%, at least 70%, at least 80%, at least 90%, or at least 95% (Z)-isomers, (all-E)-isomers, or cis-isomers, such as 5-cis- or 9-cis- or 13-cis-isomers, which have improved bioavailability relative to trans isomers. Trans isomers may isomerise into cis forms in vivo, or during storage and processing.

Carotenoid compounds, such as lycopene, for use as described herein may be natural i.e. obtained from a natural source, for example, extracted from a carotenoid-rich fruit, vegetable or other plant, such as a tomato or melon, or from fungi, algae or bacteria. In one instance, the carotenoid compound may be, or comprise, oleoresin, particularly tomato oleoresin.

A range of methods for extracting, concentrating and/or purifying carotenoids from plants are known in the art. For example, solvent extraction using ethanol, DMSO, ethyl acetate, hexane, acetone, soya or other vegetable oil, or non-vegetable oils may be employed.

Carotenoid compounds, such as lycopene, for use as described herein may be synthetic i.e. produced by artificial means, for example, by chemical synthesis. A range of methods for chemical synthesis of lycopene and other carotenoids are known in the art. For example, a three-stage chemical synthesis based on the standard Wittig olefination reaction scheme for carotenoid synthesis may be employed, in which an organic solution of Ci5 phosphonium methanesulfonate in dichloromethane (DCM) and an organic solution of Ci0 dialdehyde in toluene are produced, and the two organic solutions are gradually combined with sodium methoxide solution and undergo a condensation reaction to form crude lycopene. The crude lycopene may then be purified using routine techniques, for example by adding glacial acetic acid and deionized water to the mixture, stirring vigorously, allowing the aqueous and organic phases to separate, and extracting the organic phase containing DCM and crude lycopene with water. Methanol is added to the organic phase and the DCM removed via distillation under reduced pressure. The crude methanolic lycopene solution is then be heated and cooled to crystalline slurry that is filtered and washed with methanol. The lycopene crystals may then be re-crystalized and dried under heated nitrogen. Synthetic carotenoids, such as lycopene, are also available from commercial suppliers (e.g. BASF Corp, NJ USA).

Synthetic carotenoid compounds, such as lycopene, may comprise an increased proportion of cis isomers relative to natural carotenoid compounds. For example, synthetic lycopene may be up to 25% 5-cis, 1% 9-cis, 1% 13-cis, and 3% other cis isomers, whilst lycopene produced by tomatoes may be 3-5% 5-cis, 0-1% 9-cis, 1% 13-cis, and <1% other cis isomers. Since cis-lycopene has increased bioavailability relative to trans-lycopene, synthetic lycopene is preferred in some embodiments.

Derivatives of carotenoids as described above may be produced by chemical synthesis analogous to the synthesis described above or by chemical modification of natural carotenoids extracted from plant material.

As shown in the examples, the effects of carotenoids on the structure of a hydrocarbon, in particular lipids, are present even at low ratios of carotenoid to hydrocarbon. However, as also demonstrated in the examples, increasing the concentration of the carotenoid enhances the effect of disruption of the configuration, structural organisation, packing and interaction of the hydrocarbon molecules with each other.

According to the various aspects described above, including the particle, methods and uses, the ratio of carotenoid:hydrocarbon is between when they were added in concentrations 1:100 to 1:1,000,000. For example, the concentration may be 1:1000 to 1:1,000,000 or from 1:3,000 to 100,000.

As also shown in the example, the disruption of or changes in the lipid structure as a result of the incorporation of the carotenoid into the hydrocarbon structure can be assessed by measuring the droplet or continuum sheet size, or other formations of the lipid when it is within or on the surface of an aqueous solution.

The examples clearly demonstrate that lipid globules that have been prepared according to the methods described in the examples and have embedded in their structure a carotenoid have a larger droplet size compared to a control. For example, the increase in droplet size can be 2- to 100 fold, for example 2-, 3-, 4-, 5-, 6-, 7-, 8, 9-10-, 20-, 30-, 40, 50-, 60, 70-, 80-, 90 or 100-fold. For example, droplet size can be at least 100 μm in diameter. In another embodiment, the increase can be expressed in percentage, for example at least 10, 20, 30, 40, 50, 60, 70%.

Moreover, the disruption of the structure also results in a change of one or more of the following properties compared to a control particle that does not include a carotenoid:reduction of viscosity, reduction of density, increase in spreadability, increase of susceptibility to acid or other forms of oxidation or degradation, increase in bioavailability and clinical efficacy, reduction in digestibility, increased permeability and fluidity, increase in greasing and lubrication properties, reduction of freezing and melting time, increase in thermal conductivity, heat capacity and thermal energy storage, accelerated heating and cooling time, increase of burning time and heat generation without increased, or with reduced, fuel consumption.

In one embodiment, the modification of the hydrocarbon can be selected from one or more of the following: increase of their molecular gas, and in particular O₂, holding capacity of lipids and their ability to transport molecular oxygen, accelerated cooking time for cooking oils resulting in cooked food with increased content of vitamins and essential nutrients, formation of lipid-based or lipid-rich food, or lipid containing food, milk, cream, ice cream or beverage products, with reduced lipid digestion rate and subsequently reduced absorption of calories-rich lipids, reduction or prevention of chocolate blooming and improve chocolate taste.

An increase as used herein can be at least 1% to 100%, for example 10%, 20%, 30%, 40%, 50%, 60%, 70%, 80%, 90%. A reduction as used herein can be at least 1% to 100%, for example 10%, 20%, 30%, 40%, 50%, 60%, 70%, 80%, 90%.

In one embodiment, the modification of the hydrocarbon can be selected from one or more of the following:
acceleration of gastro-intestinal transit time for oil or fat containing food or beverages,
reduced acidic or other forms of oxidation or degradation, hence improve bioavailability and biological efficacy of essential fatty acids, phospholipids or other lipids, and embedded into them pharmaceutical, nutraceutical and other bioactive molecules, processing of which in gastrointestinal tract does not involved lipase digestion,
reduced enzymatic digestion rate for oil or fat containing food or beverages,
reduced postprandial lipidaemia and reduced absorption of calories rich lipids,
stimulation formation of molecular oxygen rich intracellular lipid droplets;
activation of mitochondria growth and respiration;
stimulation of non-shivering thermogenesis and formation of beige fat cells,
facilitating shift from glycolysis to aerobic respiration, one of the key objectives in treatment of cancer;
stimulation of assembly and/or formation of less atherogenic larger size of plasma/serum lipoproteins with increased capacity to transport molecular oxygen;
reduction of tissue hypoxia and stimulation of tissue oxygen saturation and respiration;
reduction of the sebum viscosity, facilitating its passage through skin pores, which may help to prevent or treat acne and seborrhea;
improvement of the sebum quantity and quality which can be translated into better skin lubrication, its protection from dehydration, pollution, UV-light damage and other forms of stress, and also improving skin immune defence and reduction on its surface presence of pathological or health damaging bacteria, or other microorganisms;
reduction of the viscosity of lipid-containing mucosal secretion would facilitate its passage from producing cells or glands to the surface of the lining of the mouth, throat, esophagus, stomach, intestine, colon, bile duct, eye, nose, ear, trachea, bronchi, pulmonary alveoli, fallopian tube, vagina, seminal vesicle;
improvement of the mucosal production/passage would facilitate increase in delivery of not only concomitant health beneficial molecules, for example of the immune system, but also facilitate transport of excreted/produced cells, like oocytes or sperm cells;
restoration and improvement of mucosal production will increase lubrication of these organs and protecting them from dehydration, reduce their dryness and exfoliation of the lining cells;
restoration and/or increase in sebum production and in particular enriched with carotenoids can increase presence of these molecules on the surface of the skin;
restoration and improvement of lipid-containing secretions and fluids enriched with carotenoids can increase presence of these molecules on the surface of the eyes, lining in the mouth, throat, gallbladder, bile duct, eye, nose, ear, trachea, bronchi, pulmonary alveoli, ureter, urinary blabber, urethra, fallopian tube, vagina, nose mucosae, joint cavity, synovial membrane, which would facilitate their protection from UV-radiation, pollution, chemical and physical environmental damage, reduce the dryness and exfoliation of the cells lining the surface of these organs, nourishing growth and strength of carotenoid metabolising probiotic or health beneficial bacteria, for example Micrococcus luteus on the skin;
improvements in sebum quantity and quality can also be benefit to skin cells and corneocytes, in particular to reduce their damage and the rate of desquamation, and overall slowdown and/or reverse changes in skin associated with its ageing and/or stress;
reduction of adipose tissue viscosity which could be used as priming procedure to facilitate tissues liposuction;
reduction of adipose tissues viscosity which could also be used to help to prevent and/or to manage these tissues pathological conditions such as obesity, cellulite and others;
reduce chocolate bloom;
improve or enrich chocolate and ice cream taste.

In one embodiment, the disruption of the configuration, structural organisation, packing and interaction of the molecules of a hydrocarbon with each other can be used in prevention or treatment of some physiological or pathological conditions or disease where correct folding is important, for example:
to prevent or treat constipations,
to activate non-shivering thermogenesis and formation of beige fat cells,
to enlarge the size of circulating lipoproteins and reduce their atherogenic properties,
to stimulate mitochondria growth and respiration,
to shift cellular/tissue metabolism from glycolysis to the aerobic pathway in treatment of cancer and other pathologies where aerobic pathway is depressed;
to improve molecular oxygen delivery and tissues oxygenation, as anti-hypoxia prevention, intervention, or treatment,
to improve quantity and quality of the sebum, to improve health of corneocytes, reduce or eliminate level of their clusterisation or cross-linking, to reduce rate of their exfoliation;
to prevent or to treat acne and seborrhea;
to prevent and to treat skin dryness, to improve skin lubrication and prevent its dehydration, to improve its microbiota, immunity and defence from pollution, UV and other radiation and physical and chemical factors;
to slow down and/or reverse changes in skin associated with ageing and/or stress;
to prevent and to treat skin infections;
to prevent and to treat skin inflammatory conditions;
to prevent and treat infections and inflammatory conditions or disease, associated with deficiencies of the production, or abnormalities or pathologies of lipid-containing secretions or fluids in on the surface of the eyes, lining in the mouth, throat, gallbladder, bile duct, eye, nose, ear, trachea, bronchi, pulmonary alveoli, ureter, urinary blabber, urethra, fallopian tube, vagina, nose mucosae, joint cavity, synovial membrane, which would facilitate nourishing growth and strength of carotenoid metabolising probiotic or health beneficial bacteria, for example but not limited to Micrococcus luteus on the skin, which would improve profile of microbiota in these tissues, and help to prevent or treat associated pathologies not only locally in these organs but, by releasing antibacterial, anti-infective, anti-inflammatory molecules or metabolites, which can be absorbed and be circulated around whole organism, to prevent ant treat infections or inflammatory conditions in other organs and tissues, or systemically, in the whole body;
to improve quantity and quality of the mucosal secretion and transport of secreted cells like oocytes and sperm cells;
to prevent and to treat dry eye syndrome, to improve eye surface lubrication and prevent its dehydration, to improve eye microbiota through better lubrication, immunity and defence from pollution, UV and other radiation and physical and chemical factors;
to prevent and to treat vaginal, throat and other mucosal dryness, to improve mucosal lubrication and prevent its dehydration, to improve vaginal and throat microbiota, immunity and defence from pollution, UV and other radiation and physical and chemical factors;
to reverse changes in skin and mucosa associated with ageing and/or stress,
these improvements can for example be applied to the mucosa or mucosal surface of mouth, throat, eye, nose, trachea, bronchi, pulmonary alveoli, fallopian tube, vagina, seminal vesicle.

As explained herein and demonstrated in the examples, the hydrocarbon which forms a complex with a carotenoid embedded in its structure has different physical and thus functional properties compared to a control hydrocarbon. In particular it has improved viscosity, acid or other forms of oxidation ore degradation, thermal energy conductivity and storage. Melting and/or freezing points are also modified.

Moreover, the hydrocarbon which has a complex with a carotenoid embedded has different light absorption spectrum compared to the control. This is significant as it demonstrates that a physical complex has formed between the hydrocarbon and the carotenoid, which causes a shift in the light absorption spectrum. This interaction results in conformational changes not only in the lipids, but also in the carotenoids. For example, these changes may result in appearance of a red-shift in carotenoid light absorption. In FIGS. 60 and 61, the red-shift can be clearly observed, both in lycopene and lutein absorption after they were embedded in sunflower oil in a ratio of 1:100. The red-shift is at least 1 nm or more, and/or the hyperchromism is at least 2% or more.

Another change in carotenoid conformation, as a result of formation of carotenoid-lipid complexes, can be the development of hyperchromicity. This effect develops when the same amount of molecules starts to absorb more light due to their clusterisation. In our case molecules of lutein appear to absorb significantly more light after their interaction with lipids of cocoa butter (FIG. 62). The carotenoid:lipid ratio in this experiment was 1:10. In all described experiments, the solvent was a mixture of ethanol and methyl chloride in ratio 1:5.

Thus, the particle of the invention is also characterised by a red-shift in the absorption spectrum as shown in the figures.

In one embodiment, the changes caused by formation of the complex between and a carotenpoid which is embedded/incorporated into the hydrocarbon, and a lipid in particular, can be translated into a larger size matrix of the product, which may not contain unmodified hydrocarbons, or may not contain them at all.

In another aspect, the invention relates to an emulsion comprising a particle or complex as described above.

In another aspect, the invention relates to foodstuff comprising a particle or complex as described above.

The foodstuff can be a functional or medical food or beverage, a dietary supplement, or a nutraceutical product.

In one embodiment, said foodstuff is a diary product.

In one embodiment, said foodstuff is a liquid or solid fat.

In one embodiment, said foodstuff is butter, margarine, ice cream oil shortening, lard, chocolate, peanut butter, fat based creams and spreads, milk, cream, ice cream, yogurt.

Food products can be in solid, frozen, semi-solid, gel, molten, semi-liquid or in liquid form.

For example, incorporation of a carotenoid in a dairy butter is the first step to disrupt milk fat globules, and on the second step to add to milk, cream, ice cream, yogurt or any other dairy or beverage product.

In another aspect, the invention relates to a method for disrupting the structure, e.g. folding of a hydrocarbon comprising the step of forming a physical complex of a hydrocarbon and embedding a carotenoid.

The method includes the step of melting a lipid and thoroughly mixing the lipid with the carotenoid at a temperature, which is above the melting point of the lipid. If the lipid is in a liquid state, then the blending process could be performed straight away. Further steps include measuring the droplet or continuum sheet or another structure, size, or changed lipid viscosity, or hydrophobicity, or thermoconductivity, or thermal energy storing capacity and compared to a control and/or the absorption spectrum to confirm complex formation, i.e. to ensure that the carotenoid is embedded into the hydrocarbon.

In one embodiment, the lipid has a high fat content, for example more than 50%.

The invention also relates to a product obtained by such method.

The inventor has also shown that once the lipid/fat is created, a physical complex with an embedded carotenoid is formed. It is possible to use such product to incorporate it into another matrix with lower fat content. For example, diary products such as milk or ice cream have a lower fat content than butter. Once the fat/lipid molecules of a dairy product create a complex with an embedded carotenoid, it is possible to use some of this product, such as butter as described in the examples below, and mix it with another diary product with a lower fat content (i.e. below 50%, for example milk or ice cream) to embed the carotenoid in the fat of such product. Thus, in one embodiment, the method comprises mixing a diary product with high fat content, such as butter with lipids in a physical complex with an embedded carotenoid, with a diary product with low fat content, such as milk or ice cream. The resulting low fat product includes lipids in a physical complex with the embedded carotenoid.

In another aspect, the invention relates to a use of carotenoid for disrupting the configuration, structural organisation, packing and interaction of the hydrocarbon molecules with each other.

The invention also relates to a method for measuring the disruption of the configuration, structural organisation, packing and interaction of the hydrocarbon molecules in a particle or in a matrix which comprises a hydrocarbon in its physical complex with an embedded carotenoid comprising one or more of the following steps:
a) exposing said hydrocarbon to an aqueous solution;
b) measuring changes in hydrocarbon spreadability, in terms of a droplet, continuum sheet or other formation size and their integrity;
c) measuring changes in hydrocarbon viscosity;
d) measuring changes in hydrocarbon, and/or incorporated into it, other molecules' susceptibility to acidic or other forms of oxidation, or degradation;
e) measuring changes in hydrocarbon, and/or incorporated into it, other molecules' enzymatic digestibility;
f) measuring hydrocarbon melting or boiling time;
g) measuring hydrocarbon hydrophobicity;
h) measuring hydrocarbon thermal energy storage capacity;
i) measuring hydrocarbon thermoconductivity;
j) measuring hydrocarbon heat capacity.

Skin Improvements

The observation that carotenoids have an effect on the droplet size of lipids can also be useful in therapeutic and cosmetic applications for skin improvement. As shown in the examples, carotenoids can stimulate droplet formation, which results in a boost of mitochondria and respiratory activity; they also increase the molecular oxygen capacity of serum lipoproteins and serum lipoprotein size and hydrophobicity. They also reduce sebum viscosity, which aids its outflow and skin lubrication. This leads to better skin protection from the environmental factors including bacteria present on the skin. In addition, improved sebum production can reduce skin dryness, protect corneocytes from the damage and reduce their rate of exfoliation. By providing a lubricated environment, a healthy microbiome of the skin can be maintained. Importantly, once carotenoids are ingested, they can alter the size of lipoproteins in the body by forming complexes as explained herein. This in turn leads to the advantages as described above, which include lubrication and maintaining an environment which allows the growth of certain bacteria, but also helps to prevent atherosclerosis as this is usually associated with small lipoproteins.

The applicant observed that supplementation of the skin with lycopene in middle-aged human subjects resulted in the restoration of the sebum viscosity, reduction of the corneocyte damage and desquamation, by reducing the growth of commensal skin bacteria. The applicant has also shown that lycopene can be secreted to the surface of the human body either with the cerumen, or sebum and that an increase of lycopene found in serum correlates with that found in the skin.

Thus, the invention also relates to uses of carotenoids for improving skin health, reducing sebum viscosity, stimulating sebum droplet formation in the skin, increasing the size of sebum droplets, increasing the molecular oxygen capacity of serum lipoproteins and serum lipoprotein hydrophobicity, improving skin lubrication and protection, reduction of corneocyte damage/exfoliation rate and/or reducing the amount of bacteria present on the skin. In one embodiment, the carotenoid is lycopene.

In another aspect, the invention relates to a method for improving skin health, reducing sebum viscosity, stimulating sebum droplet formation in the skin, increasing the size of sebum droplets, increasing the molecular oxygen capacity of serum lipoproteins and serum lipoprotein hydrophobicity, improving skin lubrication, reduction of corneocyte damage/exfoliation rate and protection and/or reducing the amount of bacteria present on the skin by administration of a carotenoid. In one embodiment, the carotenoid is lycopene.

In one embodiment, lycopene is administered at a dosage of 5 to 50 mg per day, for example about 20 mg per day.

In one embodiment, the rate of corneocyte exfoliation is reduced by at least 10 percent, for example at least 10 or 20%. In one embodiment, the reduction is 17%. In one embodiment, the level of their corneocyte damage, in terms of the number of the cross-linked clusters of these cells, was reduced by at least 10%, for example 20%, 30%, 40%, 50%, 60% or 70%.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments, which are described.

The invention is further described in the non-limiting examples.

### Examples

Methods—Product Preparation

In this section we describe examples of incorporation of carotenoids in different lipid or fat based food products. In most cases, we used lycopene and astaxanthin. They represent two main groups of carotenoids, carotenes and xanthophylls, respectively. In some cases molecules from the latter group were also used, Lutein and Zeaxanthin. However, the same protocols can be used for all other known carotenoids.

### Example 1

Saturated Predominantly Long-Chain Fatty Acids—Dairy Butter

Laboratory Production Method of Lycopene Dairy Butter, L-Butter

(although this is an example for the particular carotenoid and dairy butter, the same protocol can be used for other carotenoids and solid fat-based food products)

This method describes the production of 2,340 g of L-Butter dispensed as individual 30 g butter each containing 7 mg of lycopene, or 0.23 mg of lycopene embedded into 1 of butter.

Ingredients: 2,340 g unsalted butter, President, 3.640 g LycoRed Lyc-O-Mato 15% Oleoresin Ambient temperature of the production environment should be 20-21° C.

Warm the bulk stock of Lyc-O-Mato 15% oleoresin to a temperature of 40° C. and maintain at this temperature until required later.

In a suitable container melt the butter to a temperature of 45° C.±1° C. Do not exceed this temperature during the melting process. Stir the butter during the melting process. Take the bulk stock of Lyc O Mato 15% oleoresin from 40° C. incubation. Mix the Lyc-O-Mato 15% oleoresin thoroughly by rotation and inversion to ensure even mixing. Do not shake.

Carefully dispense 3.640 g of the Lyc-O-Mato 15% oleoresin on to the molten butter by pouring. Excess Lyc-O-Mato 15% oleoresin can be removed with a sterile pipette. (3.640 g Lyc-O-Mato 15% oleoresin contains 546 mg lycopene). Keep stirring thoroughly to ensure an even mixture and maintain a temperature of 45° C.±1° C. for a further 10 minutes then remove from the heat. Cool down to 35° C.±1° and then begin to dispense 30 g quantities into suitable individual moulds/containers by pouring.

Stir the mixture frequently during the dispensing process to ensure an even distribution of lycopene. Maintain the mixture at a temperature of 35° C.±1° C. during the dispensing process by carefully applying a small amount of heat. Allow the individual aliquots of 30 g butter to solidify at an ambient temperature of 20-21° C. for 1 hour.

Each 30 g of butter contains 7 mg lycopene. Storage should be in sealed containers at −20° C. up to 3 months, at +4-8° C. no more than 1 month.

Quality Control

Guarantee uniformity through out the butter mass with:
Microscopy of at least 10 fields of 800μ² (×1,000) per samples
uniformity of lycopene embedment into butter matrix
assurance no free lycopene crystals observed
HPLC—concentration of trans-lycopene 0.233 mg/1 g of butter mass, cut-off+0.02 mg.

### Example 2

Saturated Predominantly Long-Chain Fatty Acids Dairy Fat Only a Part or a Small Part of the Finished Food Matrix or Beverage.

a) Milk and Dairy Cream

Laboratory Production Method of Lycopene Milk or Dairy Cream, L-Milk or L-Cream (although this is an example for the particular carotenoid and the particular type of milk, or dairy cream, or their products, the same protocol can be used for other carotenoids and products where milk or cream are only an ingredient there, and moreover in other fat containing food or beverage products, where fat or lipids are a part, or even small part of the total mass or volume of these products; these products can be in frozen, solid, semi-solid, soft, molten, gel, gum, semi-liquid, liquid or other physical status forms).

100 ml of Cow Milk with embedded 7 mg of lycopene—3% fat.

Composition—Ratio of Ingredients:

a)

| Ingredients | Mass, in gram | % |
|---|---|---|
| Milk 0.5% fat | 100 | 99.4 |
| Dairy Butter 72% fat | 3.2 | 055 |
| Lycopene 15% oleoresin | 0.05 | 0.05 |
| | Total = 100.6 | 100 |

100 ml of Dairy Cream embedded with 7 mg of lycopene—11% fat (could be 22%, or 33%, or any other percent of fat).

Composition—Ratio of Ingredients:

a)

| Ingredients | Mass, in gram | % |
|---|---|---|
| Dairy Cream 11%, or 22%, or | 100 | 99.4 |
| 33% fat | | |
| Dairy Butter 72% fat | 3.2 | 055 |
| Lycopene 15% oleoresin | 0.05 | 0.05 |
| | Total = 100.6 | 100 |

Equipment: balances, mixer, homogeniser, spatula, thermo-controlled blender, heating plate.

Protocol
1. Warm the butter to 40° C. and soften. After that introduce oleoresin and thoroughly blend it in until complete uniformity.
2. Warm milk or cream to 50° C. Then introduce lycopene containing butter, and while thoroughly blending, heat the mixture up to up to 60-70° C. Use the homogeniser to achieve maximum uniformity.

* During prolonged storage in the fridge there is a possibility of separation of the lycopene containing fraction. Before using, this mixture can be warmed up and homogenised again.

Laboratory Production Method of Lycopene Ice Cream, L-Ice Cream

(although this is an example for the particular carotenoid and the particular type of ice cream, or ice cream based products, the same protocol can be used for other carotenoids and food or beverage products, where ice cream is only an ingredient therein)

50 gram of Vanilla Ice Cream with embedded 7 mg of lycopene—with 15% fat.

Equipment: balances, mixer, homogeniser, freezer, fridge, thermo-resistant containers, heating plate. Composition—ratio of ingredients:

| Ingredients | Mass, in gram | % |
|---|---|---|
| Milk 0.5% fat | 413 | 41.8 |
| Butter 82% fat | 17 | 1.7 |
| Dairy cream 33% fat | 360 | 36.4 |
| Dried powdered milk | 50 | 5.1 |
| Sugar powder | 140 | 14.2 |
| Vanillin | 1.5 | 0.15 |
| Gelatin | 6 | 0.6 |
| Lycopene 15% oleoresin | 0.93 | 0.09 |
| | Total = 988 | 100 |

Protocol
1. Heat dairy butter to 40° C., then introduce lycopene oleoresin and stir manually until evenly blended.
2. Heat powdered milk to 50° C. and introduce it into the lycopene-butter blend. Stir the milk-butter blend manually and heat to 60-70° C. When the blend has cooled, a small fraction of the vegetable oil from the oleoresin may appear. To avoid this, before using this blend further, it may need to be heated up and stirred using the homogeniser.
3. Introduce 6 g of gelatine into 50 g of milk (11-12%); leave the mixture at room temperature for about 1 hour to let gelatine swell.
4. Introduce into the thermo-resistant container sugar powder, powdered milk and vanillin. Slowly add the milk-butter-lycopene blend and keep manually stirring the mixture. While continuing to stir heat the blend to 50-65° C. and add swelled gelatine. Keep stirring until all ingredients are dissolved and homogenized
5. Pasteurise for 5 minutes at 80° C.
6. Place dairy cream into a separate container, cool down to 0-4° C. and whip it up using the mixer.
7. Age for 24 hours at 0-4° C.
8. Add and mix this whipped cream into the main blend (point 4), and stir it until a uniformed mass is obtained.
9. Place this new blend, “ice cream to be”, into a freezer at −5 to −7° C. Stir it every 15-20 minutes. When the blend thickened transfer into a container for freezing.
10. Hardening and short-term storage should be done at −20° C.

c) Yogurt

Laboratory Production Method of Lycopene Yogurt, L-Yogurt

(although this is an example for the particular carotenoid and the particular type of yogurt, or yogurt based their products the same protocol can be used for other carotenoids and products where yogurt is only an ingredient therein)

50 g of Yogurt with embedded 7 mg of lycopene.

Composition—Ratio of Ingredients:
b)

| Ingredients | Mass, in gram | % |
|---|---|---|
| Dairy milk 0.5% fat | 1,000 | 85.14 |
| Dairy cream 33% | 150 | 14.19 |
| Dairy butter 72% fat | 5.5 | 0.52 |
| Lycopene 15% oleoresin | 0.05 | 0.05 |
| Yogurt culture | 1 | 0.01 |
| | Total = 1,057 | 100 |

Equipment: Equipment: balances, mixer, homogeniser, spatula, thermo-controlled blender, heating plate, thermostat.

Protocol
1. Warm up the butter up to 40° C. and soften up. After that introduce oleoresin and thoroughly blend it in until completely uniform.
2. Warm cream up to 50° C. Then introduce lycopene containing butter, and while thoroughly blending, heat the mixture up to up to 60-70° C. Use the homogeniser to achieve maximum uniformity.
3. Warm milk up to 50° C. Then introduce lycopene containing butter, and while thoroughly blending heat the mixture up to up to 60-70° C. Use the homogeniser to achieve maximum uniformity.
4. Cool down the mixture to 40° C. Then add into it the yogurt culture and leave it in the thermostat at this temperature for 8 to 12 hours. Stir it up the mixture, at regular intervals as frequent as possible.
5. After that homogenise the yogurt blend until complete uniformity.

### Example 3

Saturated Predominately Medium-Chain Fatty Acids—Chocolate or Chocolate Spread

Laboratory Production Method of Lycopene Chocolate, L-Chocolate

(although this is an example for the particular carotenoid and dark chocolate, the same protocol can be used for other carotenoids and milk and white chocolate, or just cocoa butter, and also on products where chocolate is only an ingredient of other food or beverage products; the same protocol can be used for other carotenoids and oil or fat products congaing a combination of saturated, medium-chain and other fatty acids)

a) Chocolate

This method describes the production of 1000 g of lycopene Chocolate dispensed as individual 10 g chocolates each containing 7 mg of lycopene-embedded into chocolate matrix.

Ingredients: 1000 g Green & Black's Organic 85% Cocoa Chocolate

4.667 g Lycored Lyc-O-Mato 15% Oleoresin

Ambient temperature in the production environment should be 20-21° C.

Warm the bulk stock of Lyc-O-Mato 15% oleoresin to a temperature of 40° C. and maintain at this temperature until required later.

Break off a single rectangular piece of chocolate approx. 25-30 g in weight. Store this piece of chocolate in a separate container until required later.

Break up the remainder of the 1000 g of chocolate into small pieces.

In a suitable container melt the chocolate to a temperature of 48° C.±1° C. Do not exceed this temperature during the melting process.

Stir the chocolate during the melting process. When the chocolate appears to have melted completely stir thoroughly to ensure an even mixture with all chocolate melted.

Place the reserved 25-30 g piece of chocolate in an open container on a suitable balance, smooth surface of the chocolate uppermost. Tare the balance to zero. Take the bulk stock of Lyc-O-Mato 15% oleoresin from 40° C. incubation. Mix the Lyc-O-Mato 15% oleoresin thoroughly by rotation and inversion to ensure even mixing. Do not shake.

Carefully dispense 4.667 g of the Lyc-O-Mato 15% oleoresin on to the 25-30 g piece of chocolate by pouring. Excess Lyc-O-Mato 15% oleoresin can be removed with a sterile pipette. (4.667 g Lyc-O-Mato 15% oleoresin contains 700 mg lycopene).

Add the chocolate piece with the Lyc-O-Mato 15% oleoresin to the molten chocolate mixture at 48° C.±1° C. Allow the chocolate piece to melt while stirring thoroughly to disperse the Lyc-O-Mato 15% oleoresin.

Once the chocolate piece has melted continue to stir the mixture and maintain a temperature of 48° C.±1° C. for a further 10 minutes then remove the heat.

Allow the mixture to cool to a temperature of 31° C. at an ambient temperature of 20-21° C. Stir the mixture as it cools.

Quality Control

Guarantee uniformity through out the chocolate mass with:
Microscopy of at least 10 fields of 800μ² (×1,000) per samples
uniformity of lycopene embedment into chocolate matrix
assurance no free lycopene crystals observed
HPLC—concentration of trans-lycopene 0.7 mg/1 g of chocolate mass, cut-off+0.05 mg.

When the mixture reaches a temperature of 31° C. begin to dispense 10 g quantities into suitable individual moulds by pouring. Stir the mixture frequently during the dispense process to ensure even distribution of lycopene.

Maintain the mixture at a temperature of 29-31° C. during the dispense process by careful application of a small amount of heat. Allow the individual 10 g chocolates to solidify at an ambient temperature of 20-21° C. Each 10 g chocolate contains 7 mg lycopene.

Once solidified, store the chocolates away from light at 18-22° C. for wrapping.

b) Chocolate Spread

Equipment—see above as for dairy butter.

Protocol

Warm up the chocolate spread up to 40° C. and soften up. After that introduce oleoresin and thoroughly blend it by using homogeniser to achieve maximum uniformity.

### Example 4

Saturated Predominately Medium-Chain Fatty Acids—Nut Based Products

Laboratory Production Method of Lycopene Peanut Butter, L-Peanut Butter (although the example here is for the particular carotenoid and peanut butter any the same protocol can be used for other nut based products, other carotenoids and any food and beverage products where any nut products are the main, 50% or more, or only fat ingredients therein)

Equipment—see above.

Protocol

Warm up the peanut butter up to 40° C. and soften up. After that introduce oleoresin and thoroughly blend it by using homogeniser to achieve maximum uniformity.

### Example 5

Monounsaturated and Polyunsaturated Fatty Acids—Vegetable Oils

Laboratory Production Method of Lycopene Oils, L-Oils

(although here is an example for the particular carotenoid and sunflower oil, the same protocol can be used for other carotenoids and vegetable, nut, seed and fish oils; the same protocol can be used for other carotenoids and lipid products, such as Omega 3, other essential fatty acids, phospholipids, or incorporated or blended into these lipids other pharmaceutical, nutraceutical and bioactive molecules, etc.)

This method describes the production of a 50 ml of vegetable oils with embedded 7 mg lycopene. The method can be used for both sunflower, or olive, or any other vegetable, nut or fish oil which can be liquid at ambient temperature.

Ingredients: 50 ml Flora Sunflower Oil

or

50 ml Napolina Extra Virgin Olive Oil

70 mg LycoRed Lyc-O-Mato 10% Oleoresin

Ambient temperature in the production environment should be 20-21° C.

Note that olive oil may become cloudy if it has cooled to around 10° C. on storage.

Allow warming to an ambient temperature of 20-21° C. and the olive oil will clear. This may take some time. Warm the bulk stock of Lyc-O-Mato 10% oleoresin to a temperature of +40° C. and maintain at this temperature until required later. Measure 50 ml of oil in a suitable glass measuring cylinder. Transfer the oil to a suitably sized glass bottle. This bottle should be sealable as it will serve as the final storage container for the Lycopene Oil. Place the bottle containing the oil on a suitable laboratory balance. Tare the balance to zero. Take the bulk stock of Lyc-O-Mato 10% oleoresin, which has been warmed to +40° C.

Mix the bulk stock of Lyc-O-Mato 10% oleoresin thoroughly by rotation and inversion to ensure even mixing. Do not shake. Carefully dispense 70 mg of the Lyc-O-Mato 10% oleoresin into the 50 ml volume of oil on the tared balance. Take care not to exceed an addition of 70 mg as adjustments cannot be made (70 mg of Lyc-O-Mato 10% oleoresin contains 7 mg lycopene).

Note that addition of Lyc-O-Mato 10% oleoresin to the 50 ml volume of oil minimises contact of the oleoresin with the glass surface and allows mixing to be carried out more readily.

Seal the bottle and mix the oil and Lyc-O-Mato 10% oleoresin by gentle swirling and inversion. Do not shake. Complete dispersion of the Lyc-O-Mato 10% oleoresin may take some time. Once dispersed, protect the Lycopene Oil from light and allow standing overnight at the ambient temperature of 20-21° C. to allow any further dispersal to take place.

The following day mix again by gentle swirling and inversion. The Lycopene Oil is now ready for use. Store the L-Tug Oil at an ambient temperature of 18-24° C. away from light.

Mix by gentle swirling and inversion immediately before use to ensure even dispersal of the blend.

Quality Control

Guarantee uniformity through out the oil mass with:
Microscopy of at least 10 fields of 800μ² (×1,000) per samples
uniformity of lycopene embedment into oil matrix
assurance no free lycopene crystals observed

HPLC—concentration of trans-lycopene 0.233 mg/1 g of oil mass, cut-off+0.02 mg.

Analytical Methods

Microscopy

For measurement of lipid droplets and fat globules and quantification of their size binocular microscope Olympus BX41 was used with Cell\hat{ }B software for morphometric analysis. All parameters were collected from 10 randomly selected microscopic fields at ×1000).

Hydrophobicity

Principle of the Assay

There could be a number of methods to measure changes in a hydrocarbon, and in particular in the configuration, structural organisation, packing and interaction of the lipid molecules with each other. One of the main features of chemically intact but physically disturbed lipids would be certain changes in their hydrophobicity.

Therefore it would be useful to have an assay, which could help to measure these changes caused by carotenoids.

It is known that gas molecules are more soluble in hydrophobic domains than in polar structures or liquids. For example, solubility of molecular oxygen could be 4 to 10 fold greater in intact lipid membrane than in a surrounding aqueous solution [1-5]. In other words one can use the measure of soluble gas in lipids, or other carbohydrates, as a measure of its hydrophobicity. This approach seem logical, however to the best of our knowledge has never been published.

Here we describe a new method to measure hydrophobicity of lipids as a function of concentration of soluble molecular oxygen within their matrix.

There are a number of ways to measure molecular oxygen in solid, crystal and indeed lipid structures. Almost all of them are physical methods such as ESR [1] or polarography [3]. However, we decided to use much simpler and faster micellar acceleration red-ox catalymetry [6]. This test can measure O₂ in a dozen parallel samples not only lipids in model systems but also in circulating lipoproteins from human blood [7].

Results

Size of Lipid Droplets and Fat Globules

Incorporation of lycopene into the butter led to a significant increase in the size of its lipid droplets and many reached more than 100 or even 300 fold of their diameter. Statistical quantification of the changes in the lipid droplets is presented in table 1. This effect was dose-dependent, and can already be observed for less than 1 molecule of lycopene to 224,000 molecules of butter triglycerides.

This increase reaches its saturation plateau level at a ratio of 1:3,200 or above (table 1).

TABLE 1
Effect of lycopene on changes in size
of the lipid droplets of dairy butter.

| | Size of butter lipid droplets | | | | |
|---|---|---|---|---|---|
| | Number | | | | |
| Lycopene dose per | analysed | Mean* | | | |
| 30 g of butter | droplets | in μm | Min.* | Max.* | Std. Dev. |
| 0 (control) | 246 | 1.73 | 0.50 | 3.40 | 0.57 |
| 0.1 | 159 | 34.32 | 7.00 | 79.00 | 14.35 |
| 0.5 | 99 | 60.39 | 32.00 | 150.00 | 23.70 |
| 1.0 | 78 | 92.35 | 30.00 | 219.00 | 46.48 |
| 3.5 | 99 | 90.18 | 30.00 | 276.00 | 60.25 |
| 7.0 | 60 | 178.95 | 42.00 | 473.00 | 135.44 |
| 10.0 | 69 | 141.17 | 63.00 | 350.00 | 93.43 |

Similar changes were observed when astaxanthin was added to the butter shown in table 2.

TABLE 2
Effect of astaxanthin on the size of
the lipid droplets of dairy butter.

| Astaxanthin dose | Size of butter lipid droplets | | |
|---|---|---|---|
| per 30 g of butter | Mean*, in mm | SE | SD (Σ) |
| 0 (control) | 0.831 | 0.134 | 0.519 |
| 0.1 | 7.437 | 1.572 | 6.290 |
| 0.5 | 9.235 | 1.254 | 4.857 |
| 1.0 | 12.4 | 1.452 | 5.435 |
| 2.0 | 14.687 | 2.094 | 8.113 |
| 3.5 | 12.235 | 1.694 | 6.777 |
| 7.0 | 25 | 4.269 | 17.078 |
| 10.0 | 34.117 | 6.176 | 24.705 |

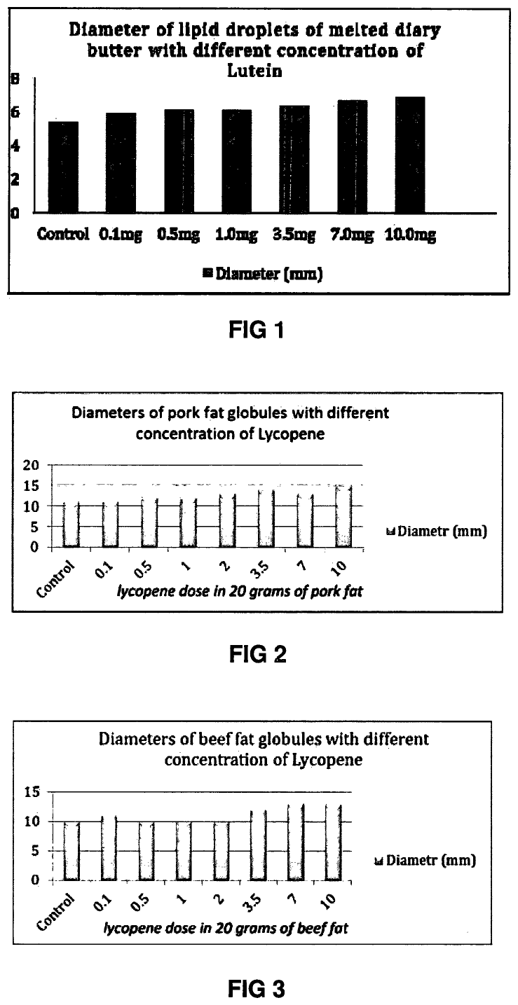

Similar changes were observed when a combination of two other carotenoids, lutein and meso-zeaxanthin, 50:50, was added to the butter, FIG. 1. Significant changes in the diameter of the lipid droplets were already observed at a concentration as low as 1 molecule of the carotenoids to 224,000 molecules of butter triglycerides.

Interestingly when lycopene was added to the dairy butter and then subsequently added into milk, or a cream the enlargement of the lipid droplets remained even after their significant dilution by an aqueous media. Moreover, this enlargement was not affected by the fermentation of the product by the yogurt culture.

Furthermore, when the lycopene containing butter was originally added to the milk and then into the blend for making ice cream, the lipid droplets in the finished project remained enlarged. This was observed for both vanilla and chocolate ice creams.

When carotenoids were added to animal fat, with a relatively high percentage of saturated fatty acid, a similar trend in increased fat globules was observed, although to a lesser degree. The maximum doses of lycopene or astaxanthin caused a similar increase in the pork fat globules diameter, only 50%, FIG. 2 and table 3.

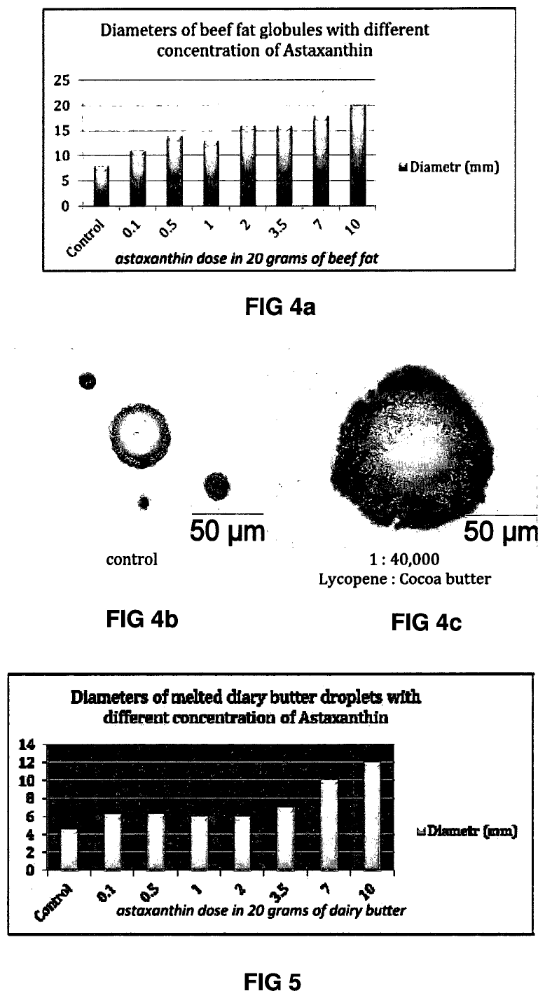

In case of beef fat, lycopene incorporation led to a smaller increase of its fat globules, only by 30%, FIG. 3. However, when astaxanthin was added their diameter increased by 2.5 folds, FIG. 4a.

TABLE 3
Effect of astaxanthin on the size of the pork fat globules.

| Astaxanthin dose per | Size of pork fat globules | | |
|---|---|---|---|
| 30 g of pork fat | Mean*, in μm | SE | SD (Σ) |
| 0 (control) | 5.25 | 0.751 | 2.909 |
| 0.1 | 5.687 | 0.798 | 3.093 |
| 0.5 | 4.937 | 0.635 | 2.462 |
| 1.0 | 5.333 | 0.704 | 2.636 |
| 2.0 | 6.812 | 0.654 | 2.535 |
| 3.5 | 8.307 | 0.952 | 3.301 |
| 7.0 | 7.9 | 1.181 | 3.541 |
| 10.0 | 8.692 | 0.748 | 2.594 |

Similar effect of enlargement of fat globules was observed when one of the carotenoids, lycopene, was added to cocoa butter, another product rich in unsaturated medium-chain fatty acids (FIG. 4b and FIG. 4c).

When lycopene was added to olive oil, rich with monounsaturated fatty acids, it increased the size of its lipid droplets in a dose-dependent manner, and table 4.

TABLE 4
Increase of Olive Oil lipid droplets size by lycopene.

| Lycopene dose | Size of Olive oil lipid droplets | | | | | |
|---|---|---|---|---|---|---|
| per 30 g of | Number of | Mean | | | Std. | Droplet |
| Olive oil | droplets | in μm | Min. | Max. | Dev. | square |
| 0 (control) | 58 | 21.40 | 12.40 | 36.60 | 8.09 | 3.69 |
| 0.1 | 17 | 46.24 | 19.30 | 75.90 | 22.43 | 27.2 |
| 0.5 | 11 | 40.00 | 15.70 | 61.40 | 13.24 | 36.36 |
| 1.0 | 18 | 185.13 | 89.00 | 413.00 | 139.59 | 10.29 |
| 2.0 | 15 | 260.80 | 126.00 | 396.00 | 110.27 | 17.39 |
| 3.5 | 19 | 457.00 | 67.00 | 988.00 | 332.63 | 24.05 |
| 7.0 | 8 | 490.50 | 205.00 | 734.00 | 205.58 | 61.31 |
| 10.0 | 10 | 367.70 | 282.00 | 469.00 | 66.81 | 36.77 |

When another carotene, β-Carotene, was added to this oil, there was a similar dose-dependent increase in the size the lipid droplets.

Addition of lycopene to polyunsaturated sunflower or canola oils resulted in a similar effect of enlargement of their lipid droplets in a dose-dependent manner, see table 5.

TABLE 5
Increase of Sunflower Oil lipid droplets size by lycopene.

| Lycopene dose | Size of Sunflower Oil lipid droplets | | | | | |
|---|---|---|---|---|---|---|
| per 30 g of | Number of | | | | Std. | Droplet |
| the oil | droplets | Mean | Min. | Max. | Dev. | square |
| 0 (control) | 221 | 93.00 | 62.00 | 126.00 | 23.53 | 0.42 |
| 0.1 | 180 | 65.16 | 43.00 | 91.00 | 13.91 | 0.36 |
| 0.5 | 121 | 144.82 | 101.00 | 199.00 | 28.90 | 1.20 |
| 1.0 | 110 | 79.73 | 56.00 | 109.00 | 14.33 | 0.72 |
| 2.0 | 113 | 149.25 | 94.00 | 298.00 | 65.42 | 1.32 |
| 3.5 | 71 | 286.00 | 105.00 | 580.00 | 192.53 | 4.03 |
| 7.0 | 59 | 202.78 | 89.00 | 325.00 | 85.15 | 3.44 |
| 10.0 | 21 | 434.81 | 118.00 | 997.00 | 284.76 | 20.71 |

The data of morphometric analysis were provided with Cell\hat{ }B programme Olympus BX41; Microscopic characteristics of oil droplets size in sunflower oil with different concentration of Lycopene. (10 randomly selected microscopic fields at x1000)

Addition of β-Carotene to the sunflower oil also caused a dose-dependent increase in its lipid droplets.

The effect of adding lycopene into another polyunsaturated oil, in this case cod liver oil, was a dose-dependent increase in the size of its lipid structures. For Olive and Sunflower oils this increase in diameter caused by lycopene could reach 20 fold, for Canola and Cod Liver oil it was 50 or 100 fold.

Like in the experiments with dairy butter, this droplet size increase can already be observed when lycopene was incorporated a ratio less than 1 of its molecule to 100,000 molecules of the fatty acids of these oils.

Hydrophobicity

The physical expansion of the size of the oil droplets observed in the above experiments was estimated by independent measurements on changes in their hydrophobicity. Data presented in table 6 show that inclusion of lycopene into sunflower oil increases the hydrophobicity of their droplets, in terms of an increase of the molecular oxygen solubility therein. With this approach it was possible to detect these changes when ratio lycopene to fatty acids reached 1 to 10,000 and then it was a dose dependent effect. This rise reached its plateau level at the ratio 1:4,000.

TABLE 6
Lycopene increase hydrophobicity of sunflower
oil in terms of oxygen gas solubility therein.

| Ratio of molecules | |
|---|---|
| of lycopene per | Hydrophobicity in terms of gas solubility, |
| molecules of fatty acids | 10⁻⁶ M O₂ per mg of sunflower oil |
| 0 | 147 |
| 1:20,000 | 143-155 |
| 1:10,000 | 180-182 |
| 1:5,000 | 197-198 |
| 1:4,000 | 275-321 |
| 1:3,000 | 248-287 |

Carotenoids Reduce Viscosity and Density of Hydrocarbons: Fats and Oils—Lubrication and Beyond

Methods to Measure Level of Carotenoid Disruption of Hydrocarbon/Lipid Folding

Methods—Product Preparation

All products used in the experiments described in this section were made as described above.

Methods and Results

### Example 6

Method—Spreadability of Carotenoid Incorporated Fat/Hydrocarbon Products on the Hard or Liquid Surface.

The described above enlargement of lipid droplets or fat globules, caused by carotenoids, translated into reduction of the viscosity of their product. This could be visually observed without any instrumentation—the larger surface area is covered by spreading a drop of oil or molten fat to lower the viscosity of this food mass.

When astaxanthin was added to the dairy butter reduction its viscosity could be visually observed After measuring and comparing the size of the spread drops this decrease was estimated to be about 3, (FIG. 5).

A similar reduction in the viscosity was observed when either lycopene or astaxanthin was added to another fat food, beef fat.

### Example 7

Method—Spreadability of Carotenoid Incorporated Fat/Hydrocarbon Products on the Surface of the Water or Other Aqueous Liquids.

1. A petri dish of 13.5 cm diameter was filled to the brim with unheated water.

2. 0.3 ml of Olive or Sunflower oil was dropped onto the surface from a standardised height (30 cm) using a 1 ml pipette.

3. The resultant droplets were photographed and measured 30 sec after impact.

4. For increased reliability, the procedure was repeated 5 times.

5. Steps 1-4 were then repeated for Olive and Sunflower oils with or without carotenoids in different concentrations.

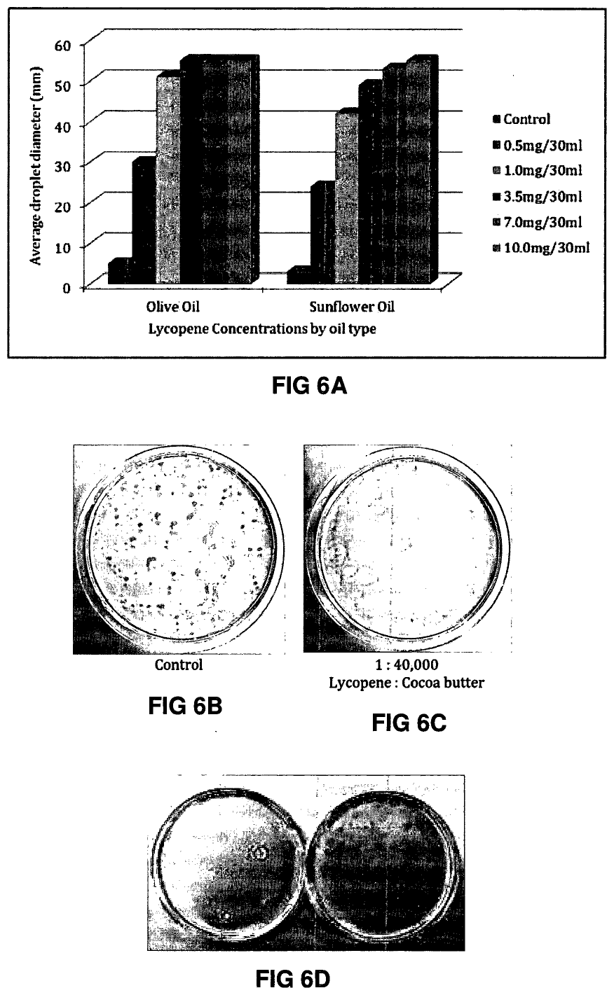

In these experiments on vegetable oils the addition of lycopene or astaxanthin caused a significantly more profound reduction in their viscosity than in the above examples on saturated fat products, FIG. 6A. For example, lycopene increased spreadability the of the olive oil on the surface of the water by 11 fold, and the sunflower oil by 23 fold. These changes were dose dependent. What was remarkable was that the sensitivity of this simple method of measuring the size of spread drops was that it was able to detect changes in the lipid folding, via changes in its physical properties when only 1 molecule of lycopene was within about 120,000 molecules of these oil lipids. The diameter of the spread on the surface of the water lipid drops of either olive or sunflower oil, with lycopene in this small ratio, was already 6-8 fold larger than the diameter drops of the intact oils.

In another experiment, when lycopene was blended into molten cocoa butter, in a molecular ratio 1 to 40,000, this resulted in solidification of the butter in a form of continuum block covering most of the aqueous surface in a Petri dish (FIG. 6C); while in the control experiment the molten butter solidified in a myriad of small droplets (FIG. 6B).

To assess a possible behaviour/spreadability of a particular lipid molecule in a stomach modelling environment 0.05M phosphoric acid—NaCl buffer with pH 2.5 was used. It was demonstrated that a single drop of Doxosahexaenoic Omega 3 acid, DHA, with 0.5% lycopene, spread instantly and created a film covering the whole surface of the buffer in the Petri dish. An application of the same size of the drop of DHA alone resulted in its re-assembly into two compact units with significantly smaller lipid surface area (FIG. 6D).

### Example 8

Method—Measuring Mass of Carotenoid Incorporated Fat/Hydrocarbon Products Needed to Cover Certain Fix Area/Surface.

The other way to assess changes in oil viscosity, caused by carotenoids, would be to measure how much oil would be needed to cover a water surface of the same size. The lower viscosity of the oil the more spreadable it will be, and consequently less of it would be needed to cover this surface. Therefore, by measuring how much oil would be required to cover a certain surface of water we can asses how deep changes in the lipid folding were made by incorporating carotenoids.

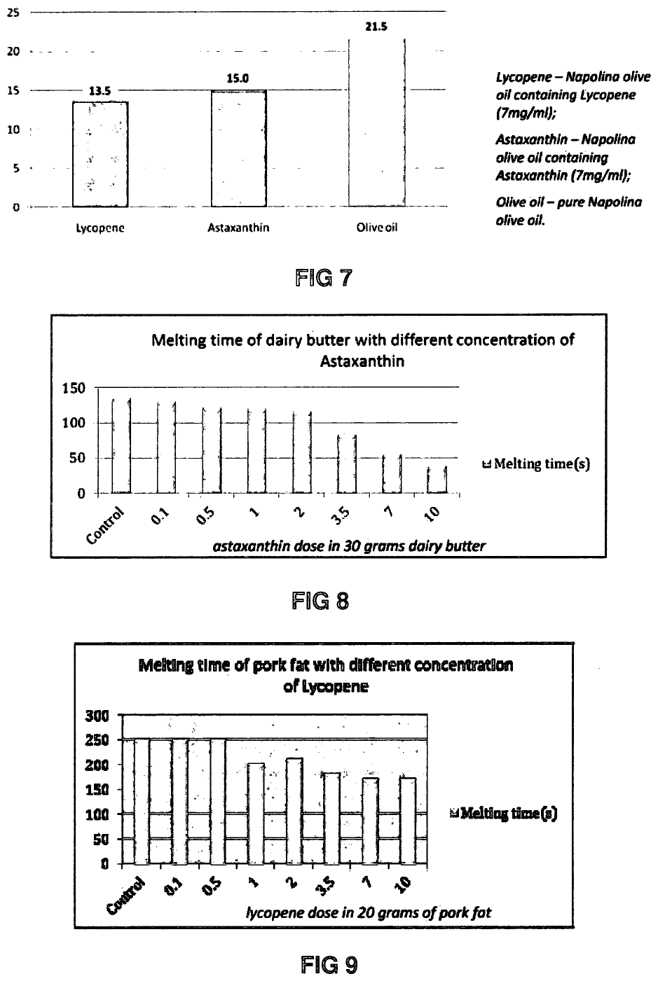

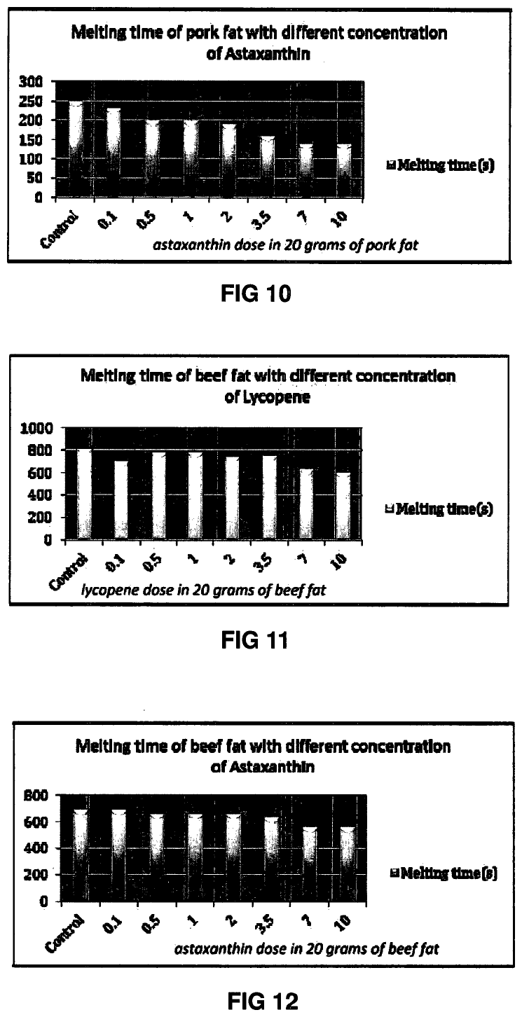

This approach was illustrated by the experimental results, which are in FIG. 7. This graph shows that for the Olive oil, when blended with astaxanthin, only 15 ml was needed to cover the surface of the water, whereas for the unmodified oil 43% more, 21.5 ml, was needed. Lycopene incorporation caused even deeper disruption of the lipids of the oil, meaning that almost 60% less of it is required to cover the same water area.

Measuring changes of the viscosity through the spreadability could be applied not just for liquid hydrocarbons but other liquids, including aqueous ones, for example, but not limited to, lipid suspensions, emulsions, droplets or micelles.

In our experiment it was shown that the effect of the reduction of the viscosity of dairy butter, caused by lycopene incorporation, remained even after its particles were significantly diluted and blended in the milk or cream. It was interesting to notice that the changes of the physical properties in the small component of the products, for example milk where fat was only 3%, affected the viscosity of the whole products. The size of the drops of the milk and cream, which spread over the glass surface, were noticeably larger for the products where fat was modified with lycopene than the control unmodified products. Moreover, this effect was not affected by the fermentation of the product by the yogurt culture.

In these experiments we also noticed that the disruption of lipids with lycopene resulted in changes to other physical properties, independent from viscosity—such as their density and lipid fractions containing lycopene were lighter than those without it.

### Conclusions

Reduction of the viscosity and the density of fats and oils with different profiles and concentrations of saturated and unsaturated fatty acids indicate that changes in configuration, structural organisation, packing and interaction of lipid molecules with each other made by carotenoids could be used across broad range of hydrocarbons.

Applications of these new properties can be used but not limited to:
to increase spreadability of oil, butter, margarine and other fat based food products;
to increase spreadability of food or beverage products where fat is only a part of them;
to increase spreadability and permeability of industrial solid fat based products;
to increase permeability and spreadability of industrial liquid oil or other hydrocarbon based products;
by increasing spreadability and permeability of industrial fats, or oil or other hydrocarbon based products their utility properties such as lubrication or greasing for any purposes, including diminishing friction of mechanical parts, or other technical, transport, engineering or other applications can be improved;
to reduce/save/economise the use of edible fat or oil in cooking practice when less viscous and lower density material would be required to cover surface of cooking utensils or surface of the cooking food;—this would reduce the quantity of the fat or oil based products used for these purposes, hence reducing the cost of the cooking products and the cost of the cooking process itself, and as a result of this to reduce the cost of the cooked meal;
to reduce/save the use of industrial fat, or oil or other hydrocarbon based products when less viscous and lower density material would be required to cover the same surface for lubrication or greasing for any purposes, including diminishing friction of mechanical parts, or in other technical, transport, engineering or other applications;—this would reduce the quantity of the fat, or oil or other hydrocarbon based products used, hence reduce their cost and the cost of processes when they were used, and as a result of this to reduce the cost of the products when these hydrocarbons will be applied;
to reduce viscosity and/or lower density of biodiesels or other hydrocarbon fuels which would lead to mitigation of wear of fuel pumps and injectors, this would improve the structure of the fuel spray coming out of the injectors, decrease spray angle and increase spray penetration, which would improve efficacy, expand life time and reduce cost of the engine or machinery using fuels modified by carotenoids.

Carotenoids Reduce Melting Time and Defrosting Time of Hydrocarbons: Fats and Oils—from Food to Grease, Antifreeze and Fuel

Methods—Product Preparation

All products used in experiments described in this section were made as described above. Methods

### Example 9

Melting Time

Approximately 20 mg of butter, or pork fat, or beef fat, or cocoa butter, or chocolate, with or without certain concentrations of carotenoids, were placed on the surface of the laboratory slides and incubated at 37° C. in a laboratory incubator (TLK39).

### Example 10

Defrosting Time

To determined defrosting time, frozen oil aliquots of 200 μI were incubated at ambient temperature of 20±2 C° until they were completely melted. Period of time, which was required for the tested sample to be melted, was measured with a laboratory timer (QUANTUM).

Results

Melting

The above described enlargement of lipid droplets or fat globules, caused by carotenoids, translated into other physical changes of the tested products, reduction of melting time and defrosting time. Incorporation of astaxanthin into dairy butter led to accelerating melting process in a dose dependent manner. At its concentration of 0.3 mg per 1 g of butter the melting time reduced from 140 seconds to 38 seconds, more than 3.5 fold, FIG. 8.

Incorporation of lycopene or astaxanthin into pork fat also resulted in a dose dependent reduction of its melting time, FIG. 9, 10. Interestingly, these changes were already registered at a ratio of a number of molecules of astaxanthin to a number of these fat molecules in 1 to 100,000.

Similarly, although to a lesser degree, this dose dependent effect of carotenoid was observed for the beef fat too, FIG. 11, 12.

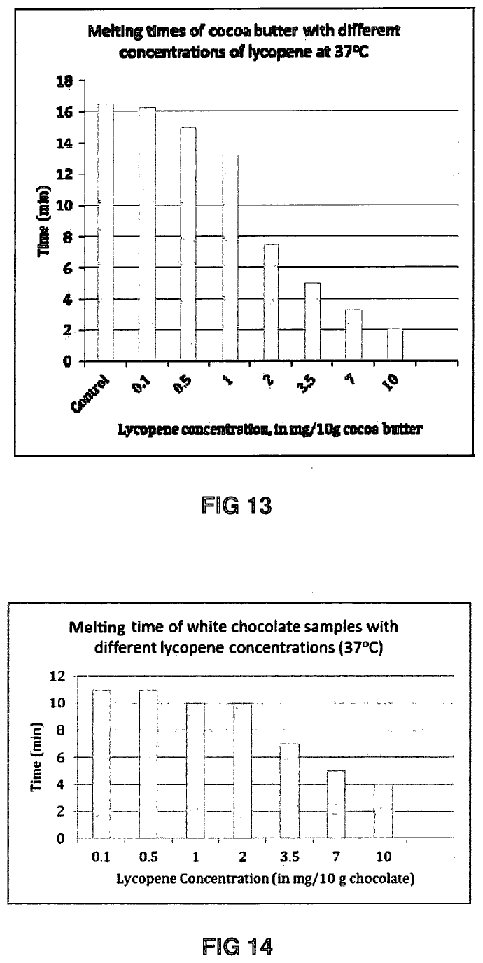

A dose depended acceleration of melting time of cocoa butter by incorporating lycopene is presented in FIG. 13. At its concentration of 0.3 mg per 1 g of this butter the melting time reduced from 16.5 minutes to just 2 minutes, more than 8 fold.

Similar significant changes in the melting time were observed in cocoa butter based products such as white, milk and dark chocolate. Results presented in FIG. 14 show dose-dependent reduction of the melting time of white chocolate by incorporating lycopene. At its concentration of 0.3 mg per 1 g of the chocolate the melting time reduced from 11 minutes to 4 minutes, just under 3 fold.

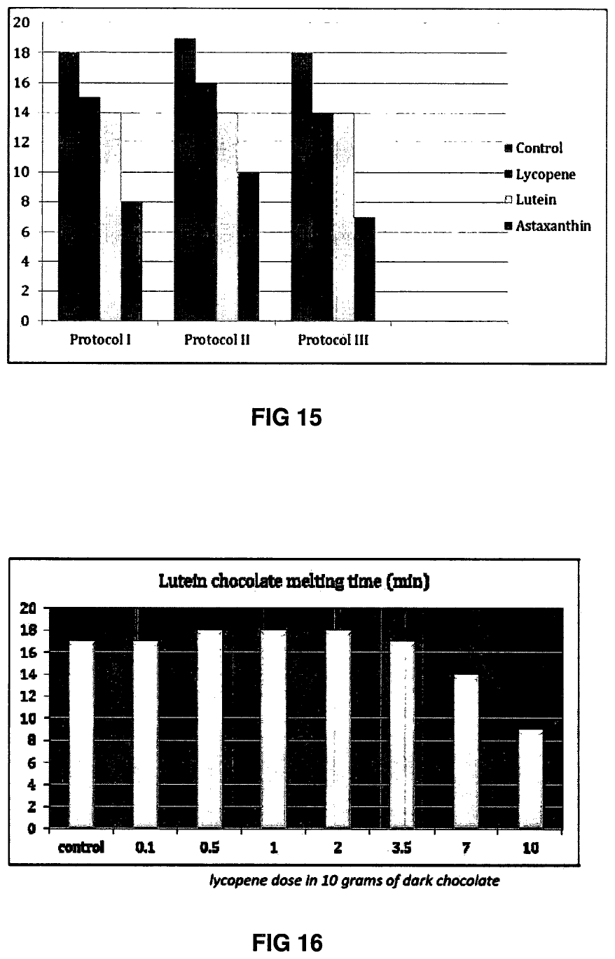

Results presented in FIG. 15 show three different carotenoids—lycopene, lutein and astaxanthin, can reduce, albeit to a different degree, the melting time of dark chocolate regardless of its tempering protocols: Protocol I—28° C., Protocol II—29° C., Protocol III—30° C.

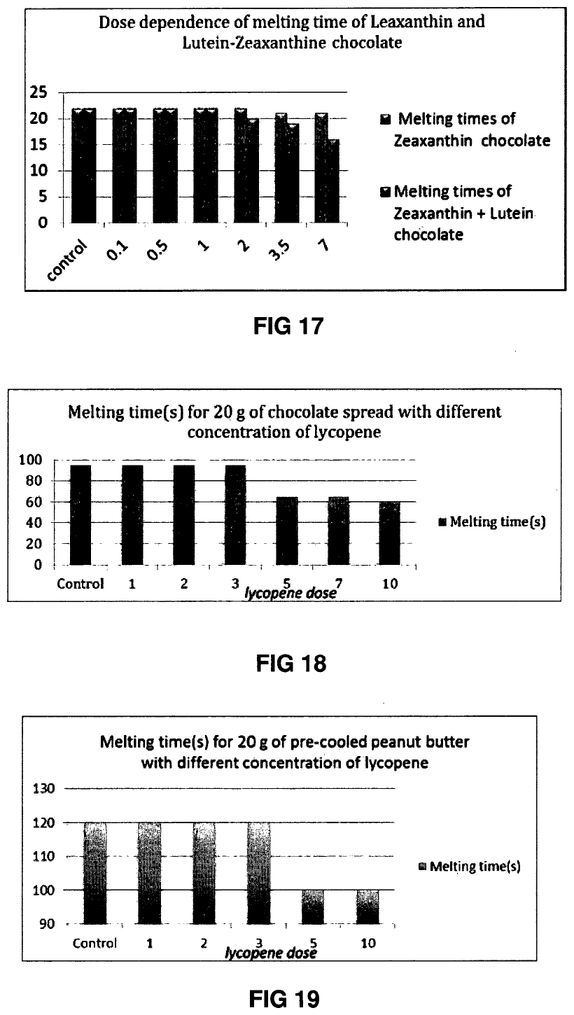

Dose dependent effect of these carotenoids on changes of the meting time of dark chocolate is presented in FIG. 15. At a concentration of 1 mg per 1 g of the chocolate the melting time reduced from 19 minutes to 8 minutes for lycopene, 6 for lutein and 5 for astaxanthin. This is further shown in FIG. 16 (lycopene dark chocolate) and FIG. 17 (Zeaxanthin and Zeaxanthin+Lutein chocolate).

Control of the melting time of chocolate is an important physical factor especially valued by consumers, hence by the chocolate industry. The faster chocolate melts the more enjoyable the ‘mouth experience’ and also the better/richer chocolate taste.

Interestingly, the changes in the melting time of chocolate caused by carotenoids were also observed in products where chocolate was not the only ingredient.

For example, in experiments with the chocolate spread, when the lycopene was 0.25 to 0.5 mg per 1 gram of this product its melting time reduced by 30-35%, FIG. 18.

Moreover, this phenomenon was not limited to chocolate or chocolate containing products. In a completely different fat containing product we observed a similar effect with the addition of lycopene into peanut butter resulting in a reduction of its melting time, FIG. 19.

Results

Chocolate Blooming

Milk chocolate is typically a blend of two different fats, cocoa butter and milk. Cocoa butter itself, hence dark chocolate, contains a mixture of different triglyceride fractions. Different fats and different fractions of the same origin fat have different melting temperature and melting rates. As a result of this during storage or temperature stress chocolate develops so call “blooming”. This occurs when chocolate, after been partially melted, starts to cool down. During this process different triglycerides would start to solidify at different rate, which would lead to their phase separation and appearing different fat fractions on the chocolate surface and within its matrix.

This would negatively change not just appearance of the chocolate but its taste too.

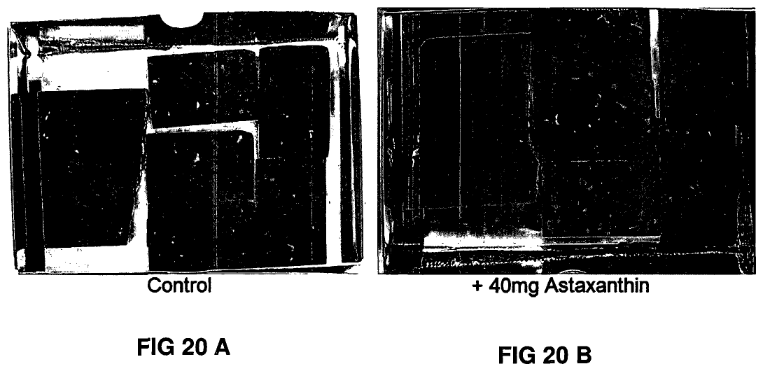

In our experiments we noticed that reducing the melting time of cocoa butter either in a free form or as part of milk or dark chocolate it was possible to reduce or prevent blooming. In one of our experiments we incubated a dark chocolate sample with or without embedded astaxanthin. Both of these samples were with 15% coarse hazelnuts. After incubation for 3 months at 28° C., and then for 3 days at 20° C., we observed appearing chocolate bloom. As it shown in FIG. 20A-B, the chocolate with blended in astaxanthin it seems was more resistance to these changes in cocoa butter viscosity than in the control sample.

Moreover, melting in the mouth is an important parameter for consumers, and therefore in the chocolate industry to manufacturers. The faster and smoother it melts the better not only the so called “mouth experience” but chocolate taste too. This observation indicates that embedding carotenoids into the chocolate matrix favoured formation of smaller chocolate crystals, which is known to interact differently with taste receptors compared to larger, coarser ones. The former interaction leads to a perception of richer chocolate aroma and taste, and the latter results in less aroma, more bitter and a worse taste.

Results

Defrosting

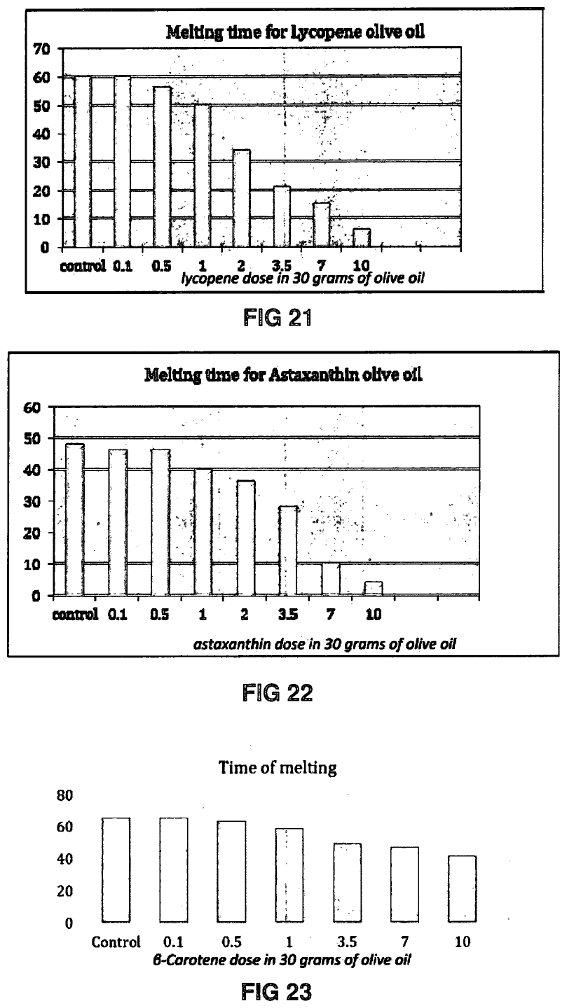

Results presented in FIG. 21 shows that the incorporation of lycopene into Olive oil can significantly accelerate its thawing/defrosting time. At its concentration of 0.3 mg per 1 ml of the oil the thawing time reduced from 60 seconds to 6 seconds, i.e. 10 fold. For astaxanthin, for the same concentration, this reduction was 11 fold, from 48 seconds to 4.3 seconds, FIG. 22. β-Carotene could also accelerate defrosting olive oil, although not to the same level as lycopene or astaxanthin, FIG. 23.

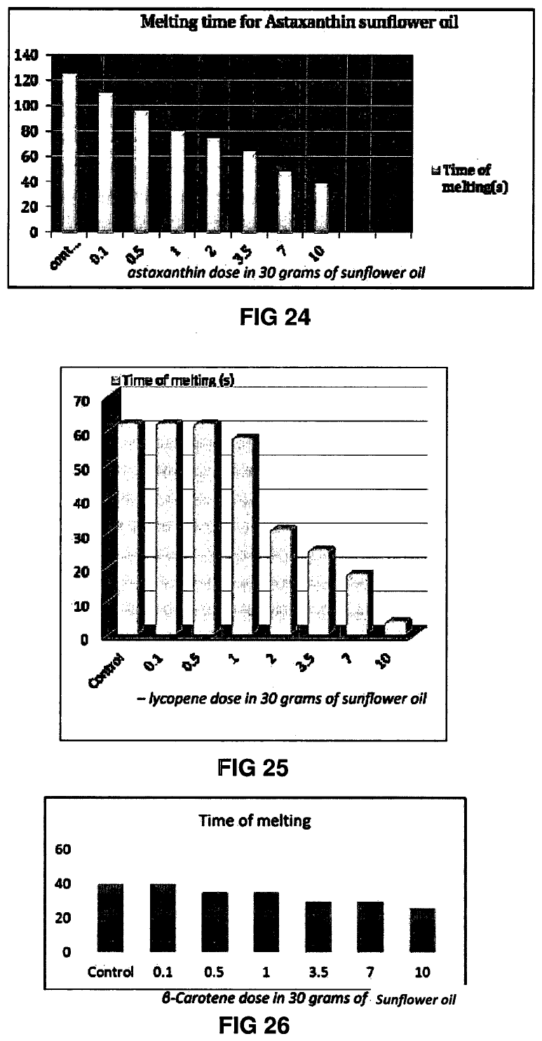

Similar effects of carotenoids were observed for Sunflower oil. For three carotenoids, astaxanthin, lycopene and β-carotene, there was a dose dependent reduction of the thawing/defrosting time, FIG. 24

FIG. 26 Astaxanthin at concentrations of 0.3 mg per 1 ml the oil reduced the thawing time by just above 3 fold, and lycopene in the same concentration by 17 fold.

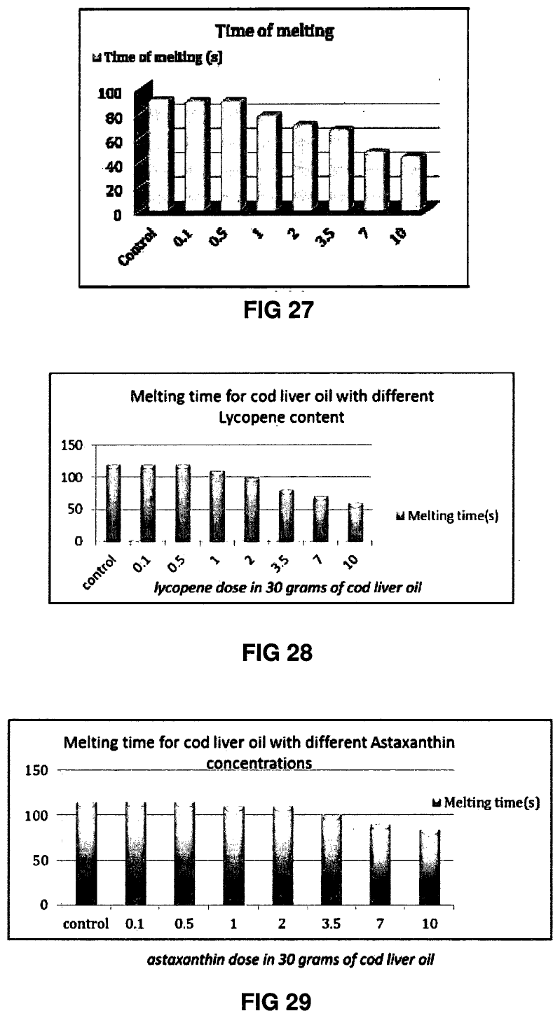

Incorporation of lycopene into Canola/Rapeseed also resulted in a similar dose dependent reduction of thawing/defrosting time. At its concentration of 0.3 mg per 1 ml of the oil the thawing time reduced from 85 seconds to 40, just over 2 fold, FIG. 27.

Incorporation of lycopene into another oil rich with polyunsaturated fatty acids, Cod Liver oil also resulted in a similar dose dependent reduction of thawing/defrosting time. At its concentration of 0.3 mg per 1 ml of the oil the thawing time reduced from 120 to 55 seconds, FIG. 28.

Incorporated astaxanthin had a similar effect, although not so prominent as above, on thawing of Cod liver oil, FIG. 29.

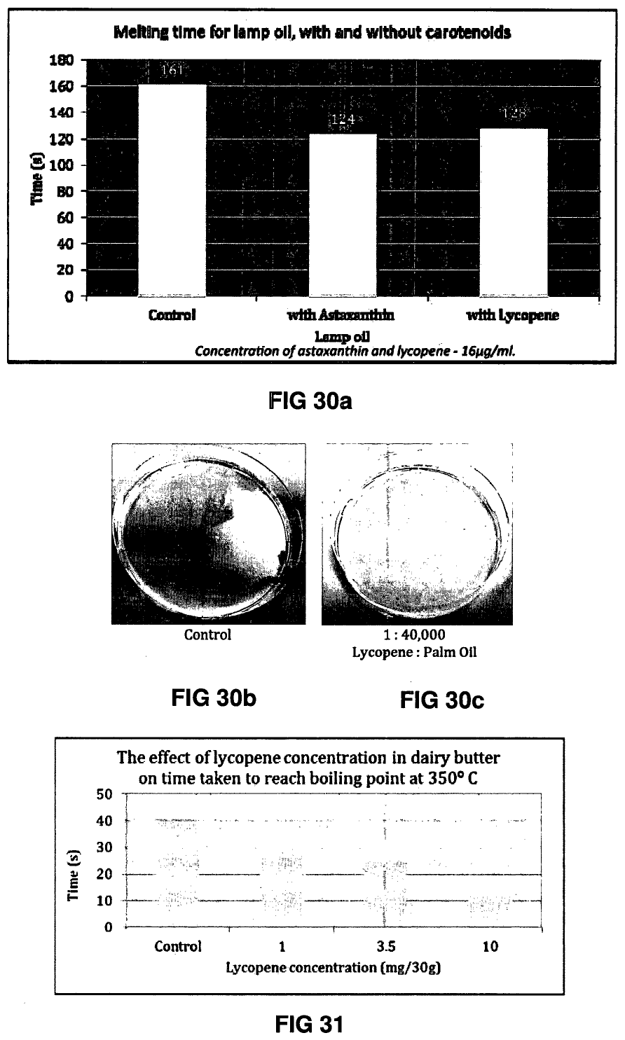

In FIG. 30A it is shown that addition of carotenoids such as astaxanthin or lycopene to kerosene or paraffin/lamp oil, an organic hydrocarbon product, could reduce its melting time. This effect was observed even when 1 molecule of either of these carotenoids was added to almost 180,000-190,000 molecules of this oil.

Anti-Freezing/Preventing Solidification

In an opposite experiment, a carotenoid was used to prevent freezing/solidification of a hydrocarbon. When a droplet of molten at +40° C. palm oil was placed on the water surface it solidified in a single unbroken floating block (FIG. 30B). However, when lycopene was blended into molten palm oil, in a molecular ratio 1 to 40,000, and its droplet of the same mass, as in the previous experiment, was place of the surface of the water it solidified and big number of separate broken particles (FIG. 30C).

### Conclusions

Reduction of the melting and/or defrosting time of fats and oils with different profiles and concentrations of saturated and unsaturated fatty acids indicates that the hydrocarbon folding disruption by carotenoids could be used across a broad range of products and applications.

This also means that carbohydrate products could be kept at a liquid state at lower temperatures, and carotenoids could be considered as a sort of antifreezes.

These properties can be used but are not limited:
to reduce viscosity and/or increase spreadability of oil, butter, margarine and other fat based or fat containing food products in lower/cooler temperatures, cooler seasons and cooler climates;
to reduce viscosity and/or increase spreadability and permeability of industrial solid fat based products in lower/cooler temperatures, cooler seasons and cooler climates;
to reduce viscosity and/or increase permeability and spreadability of industrial liquid oil or other hydrocarbon based products in lower/cooler temperatures, cooler seasons and cooler climates;
by reducing viscosity and/or increasing spreadability and permeability of industrial fat, or oil or other hydrocarbon based products to improve their utility properties such as lubrication or greasing for any purposes, including diminishing friction of mechanical parts, or other technical, transport, engineering or other applications in lower/cooler temperatures, cooler seasons and cooler climates;
by preventing freezing or accelerating defrosting of industrial fat, or oil or other hydrocarbon based products to improve their utility properties such as lubrication or greasing for any purposes, including diminishing friction of mechanical parts, or other technical, transport, engineering or other applications;
to prevent freezing or accelerate defrosting of fat, or oil or other hydrocarbon based food products;
to reduce melting time of chocolate, hence to reduce or prevent its blooming and improve its taste and mouth experience;
to reduce viscosity and/or increase spreadability and reduce melting time of therapeutic, cosmetic or personal hygiene oils, ointments, creams, waxes, suppositories, etc.

Carotenoids Increase Thermal Conductivity and Thermal Energy Storage Capacity of Hydrocarbons: Fats and Oils

The physical expansion of the size of the oil droplets observed in above experiments should result in increase of their thermal conductivity and heat/thermal energy storage capacity of oils, fats and other hydrocarbons.

### Example 11

Heating Dairy Butter—Design of the Experiment.

Small pieces of butter (about 1 g) were placed in a metallic tray. They were heated to 350° C. (Revotherm, Scientific Instruments Ltd.) and boiled for 3 minutes. Time taken to reach boiling point was fixed with a laboratory timer (QUANTUM).

In this experiments it was shown that addition of one molecule of lycopene to more than 22,000 molecules of dairy butter triglycerides reduced timing to bring this butter to its boiling point by 25%. When the number of lycopene molecules was increase to 10, the time reduced by 75%, FIG. 31.

### Example 12

Heating Oil-in-Water Emulsions

All experiments involving heating were carried out in the laboratory using SHC-I Mycrocrystal Top Stirrer Hotplate (Maplelab Scientific) that allows precise setting of hotplate temperature in the range between 20° C. and 550° C.

An additional nine heating experiments were carried out when either pure Napolina olive oil, Napolina olive oil containing Astaxanthin (7 mg/ml), or Napolina olive oil containing Lycopene (7 mg/ml) was carefully poured over the surface of the water in order to prevent evaporation. The amount of oil required to completely cover the surface of the 100 ml water sample in the cup (diameter—7.4 cm) was measured (15 ml Falcon laboratory tubes and 1 ml Pipetman pitettes were used) and recorded for each oil type. The thickness of the resulting oil film (necessary to completely cover water surface) was then calculated. Temperature change curves were compared for uncovered water and water samples overlayed with three varieties of oil at heating temperatures of 200° C., 250° C. and 300° C. Water samples overlaid with oils were heated until water reached boiling point (usually at 99° C.). Temperature measurements and result recording were done as described above.

Repeat protocol at least three times and record average temperature readings after each time period for each oil type.

Addition of carotenoids to fats or oils not only increases their own thermal conductivity but also their emulsions in aqueous solutions.

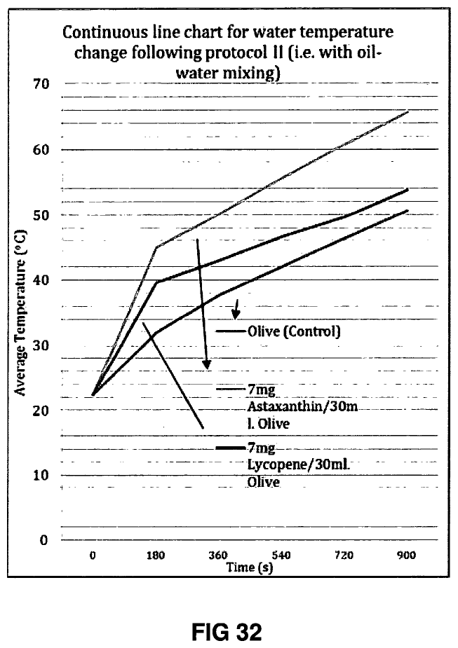

For example, the addition of 1 molecule of lycopene to 67,000 molecules of fatty acids of olive oil increased the rate of the heating of its emulsion in water, 50%:50%, from 10° C. to 18° C. in the first 3 minutes. The addition of 1 molecule of astaxanthin to 71,000 molecules of the fatty acids in the same water emulsion, increased this rate 10° C. to 23° C., FIG. 32.

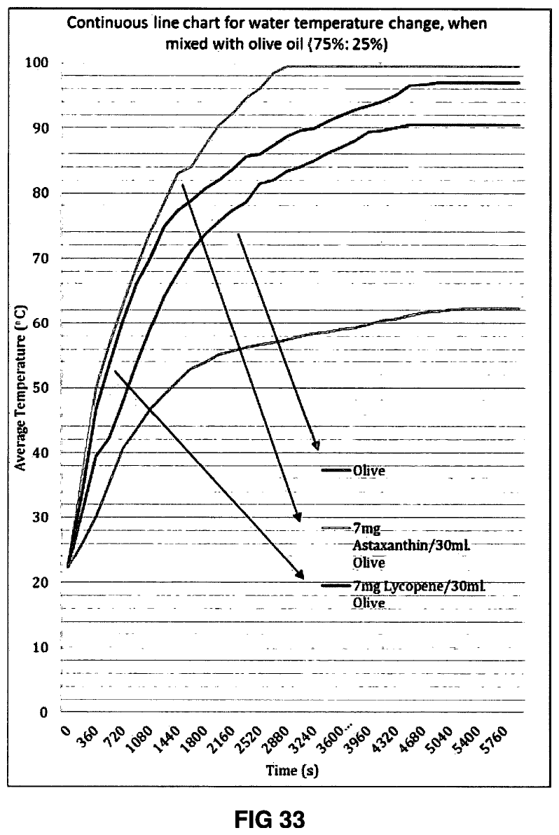

In the experiment, when the ratio of water to olive oil was 75%:25%, addition of astaxanthin to the oil, in the same concentration as in the previous example, not only accelerated its heating time but allowed a significant increase in the maximum temperature which this emulsion could reach, from 62° C. to 90° C. Addition of lycopene resulted in this maximum temperature reaching 100° C., FIG. 33.

### Example 13

A Complex of Hydrocarbon with a Carotenoid can Store and Release Thermal Energy.

Step 1. Formation of different types of hydrocarbon—carotenoid complexes at room temperature:
an oil/lipid complex with lycopene,
an oil/lipid complex with lycopene in emulsion with water;
a non-lipid hydrocarbon complex with lycopene,
a combined oil/lipid and a non-lipid hydrocarbon complex with lycopene with or without water.

Step 2. Wrap them in foil or other material preventing oxygen or any gas diffusion.

Step 3. Freeze the preparations above at for example—18° C. for 24 hours.

Step 4. Carefully return these preparations back to room temperature.

Step 5. Measure and observe the released accumulated thermal energy.

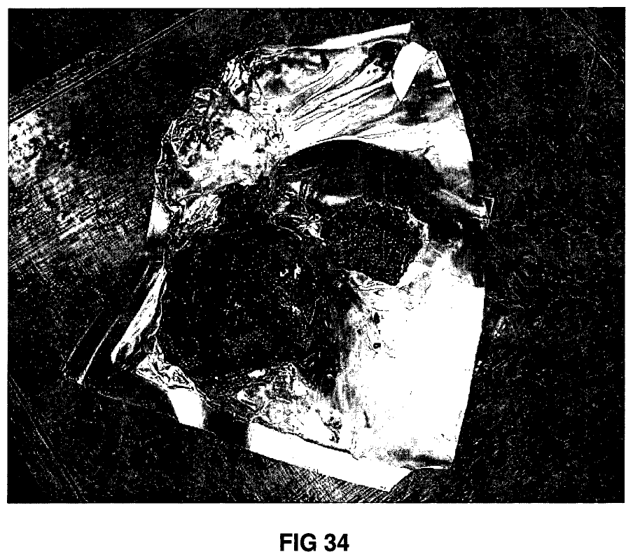

This release was rapid and resulted in an explosion; charcoal remains of the product containing hydrocarbon-lycopene complex can be seen on FIG. 34.

(It should be noted that the disclosed principles and steps of this thermo-cycle experiment is only an example, which could be modified, diversified, optimised and adapted for different applications of different hydrocarbons, lipids and carotenoids)

### Conclusion

Observed ability of hydrocarbon—carotenoid complexes accumulate, store and release thermal energy could be used for a number of practical application.

Carotenoids Accelerate Heating Time of Oils and Fats: Reduction of Cooking Time—Increase of Nutritional Value of Cooked Meal

Increased thermal conductivity of oil or fats, for example, may reduce time for cooking when these products are used.

Cooking Experiments

### Example 13

Cooking Chicken Breast
1. Weigh out a chicken breast sample of 150 g (if appropriate, exact weight can be altered depending on the meat in question, as long as the mass of all samples within any one category of meat is identical).
2. Douse the sample in the control (olive oil), ensuring that oil is in contact with the entire surface of the sample.
3. Pre-heat oven for 900 s to a temperature of 275° C.
4. Using probe thermometer, take digital reading of initial temperature of sample from its thickest point.
5. Place sample in oven for 300 s.
6. Remove sample and take digital thermometer reading from the same spot. Record the reading.
7. Repeat step (5).
8. Repeat step (6).
9. Repeat step (5).
10. Repeat step (6).
11. Now that the sample has been cooked for 900 s, switch off the oven. If the protocol has worked smoothly, sample temperature should exceed safety temperature at this point. The exact point at which the requisite temperature was reached can be attained by graphical extrapolation.
12. Record results for sample and tabulate data on excel spreadsheet.
13. Repeat steps (1-12) for sesame oil, 7 mg Astaxanthin/30 ml olive oil, and 7 mg Lycopene/30 ml olive oil. Record all results.
14. Repeat steps (1-13) three times for each oil type, taking averages of results and constructing graphs as appropriate for further analysis and graphical extrapolation techniques.

### Example 14

Cooking Salmon File

Note that this is based on the protocol formulated in the previous experiment. However, after extensive calibration certain masses have been altered and time lengths have been reduced to compensate for the lower target temperature required for a fish sample to be considered “cooked” (i.e. 63° C., and not 83° C. as required for chicken):
1. Weigh out a fillet sample of 110 g (salmon), or 120 g (tuna).
2. Douse the fillet sample in the control (olive oil), ensuring that oil is in contact with the entire surface of the sample.
3. Pre-heat oven for 900 s to a temperature of 275° C.
4. Using probe thermometer, take digital readings of initial temperature of the fillet sample from its thickest point.
5. Place sample in oven for 180 s.

Other steps are the same as in above experiment.

### Example 15

Cooking Marinated Lamb

To guarantee absolute methodological standardization, the protocol employed for cooking each marinated lamb leg steak was identical to the protocol used above. The marinating process was carried out as follows:
1. Weigh out four lamb leg steak samples of 90 g.
2. Place each sample in a sterile plastic marinating container.
3. Pour 60 mg of the oil being tested (Olive, Sesame, 7 mg Astaxanthin/30 ml Olive, 7 mg Lycopene/30 ml Olive) over each sample, so that it is fully submerged.
4. Affix lids to the marinating containers, pressing down firmly on the lamb leg steak and oil inside.
5. Leave for 120 minutes to ensure that the oil can soak into the lamb leg steak, meaning that each sample is adequately marinated in each respective oil type.
6. Remove samples from marinating containers and follow protocol recorded as in previous experiments.

### Example 16

Influence of Lycopene and Astaxanthin Added to Olive Oil on Vitamin B12 Preservation/Loss During Chicken Liver Cooking with and without Lemon Juice.

The purpose of this work package was to assess preservation/loss of Vitamin B12 during chicken liver cooking when the livers were cooked either with or without the addition of lemon juice. For this reason it was decided to check the possible influence of Lycopene and Astaxanthin on Vitamin B12 preservation during chicken liver cooking.

Vitamin B12 concentration was determined in chicken livers cooked under different conditions (addition of water, pure olive oil, olive oil containing Lycopene and olive oil containing Astaxanthin) as in the previous experiments. Two cooking modifications were performed at the same time; in one of them the livers were prepared without adding lemon juice; in the other one lemon juice was added. The experiments were repeated (cooking experiments 1 & 2) in order to provide two independent results for each measurement point in order to achieve higher result reliability.

Methodology

Cooking Experiments

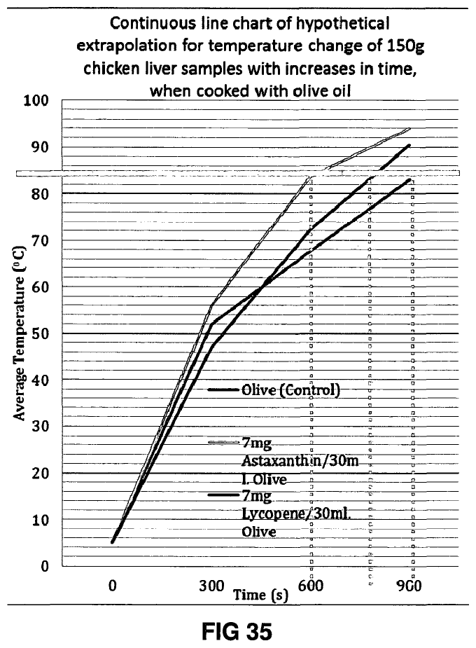

Whole chicken livers (weight between 25 and 35 g, see FIG. 35) sourced from Sainsbury's (Sainsbury's British Fresh Chicken Liver 400 g) were oven-cooked (oven temperature 180° C.) in individual small aluminium foil containers (one liver per container). The following preparation conditions were applied before cooking: 1) Addition of 15 ml of Napolina olive oil containing Lycopene (7 mg/ml); 2) Addition of 15 ml of Napolina olive oil containing Astaxanthin (7 mg/ml); 3) Addition of 15 ml of pure Napolina olive oil; 4) Addition of 15 ml of water; 5) Addition of 15 ml of Napolina olive oil containing Lycopene (7 mg/ml) and 3 ml of fresh lemon juice; 6) Addition of 15 ml of Napolina olive oil containing Astaxanthin (7 mg/ml) and 3 ml of fresh lemon juice; 7) Addition of 15 ml of pure Napolina olive oil and 3 ml of fresh lemon juice; 4) Addition of 15 ml of water and 3 ml of fresh lemon juice. 300 mg of salt was added to each chicken liver before cooking. Compared to previous experiments with salmon and mammalian (calf's and lamb) liver, olive oil, water and lemon juice amounts as well as added salt amount were reduced by 40% due to smaller sized chicken livers compared to previously tested products.

Livers were turned several times in order to make sure that they were completely covered with the added liquid following oil/water and lemon juice addition respectively. During the cooking process measurement of the internal (doneness) temperature of the livers was performed using a digital food thermometer at the following time points: 3 minutes, 6 minutes, 9 minutes, 12 minutes, 15 minutes and 18 minutes. Temperature measurements for each time point were registered and recorded.

All containers had to be taken out of the oven (together with the supporting oven wire rack) for temperature measurements; hence only times when the livers were in the oven was counted as cooking time. Once the internal temperature of the liver reached 75° C. (doneness temperature for chicken liver), a small (between 0.3 g and 0.5 g) fragment of liver tissue was carefully (special attention was always paid to avoiding simultaneous collection of cooking liquids) taken from the relevant liver (using a scalpel blade and thumb forceps) and immediately placed in a 15 ml laboratory tube containing 1 ml of sample dilution buffer (PBS from Vitamin B12 BioAssay ELISA Kit, see below). An additional sample of fresh chicken liver was also taken before cooking experiments.

Chicken Liver Sample Preparation

Tubes containing samples were transferred to the laboratory and weighed using analytical scales (Discovery DV114C, OHAUS Corp.). Sample dilution buffer was added to each sample to provide the ratio of 19 ml of sample dilution buffer per 1 g of chicken liver sample. Following the addition of the dilution buffer samples were homogenised using IKA T10 basic Ultra-Turrax homogeniser system at maximum speed (30,000 RPM). After homogenisation of each sample the homogeniser was disassembled and both its rotor and stator were carefully cleaned in order to prevent sample cross-contamination. Following homogenisation of all samples, the obtained homogenates were centrifuged for 10 minutes at RCF=1920 g (4500 RPM) using Hettich Universal 320 laboratory centrifuge. 600 μl of the resulting supernatant from each tube was transferred into pre-labelled individual Eppendorf tubes for Vitamin B12 concentration determination. Sample homogenisation and supernatant preparation were completed within 24 hours following each cooking experiment. Before analysis the supernatants were stored at 4° C.

Vitamin B12 Concentration Determination

All samples were analysed within 48 hours following cooking experiments. Vitamin B12 BioAssay™ ELISA Kit (US Biological) was used for Vitamin B12 concentration determination. Vitamin B concentrations in the samples from the cooking experiment were determined in both undiluted supernatants (1 g of liver+19 ml of buffer) and dilutions 1/2, 1/4 and 1/8 (the latter only for the samples cooked without lemon juice). The dilutions were prepared using sample dilution buffer (PBS) supplied with the kit. B12 concentration evaluation was performed in 50 μl of solution according to the protocol provided with the kit.

Vitamin B12 concentration determination was performed using Multiscan FC microplate photometer (Thermo Fisher Scientific) by measuring optical light absorbance at 450 nm (reference wavelength 620 nm) as recommended by the kit manufacturer. All calibration standards were measured in duplicates. Measurement results were analysed using Skanit software for Multiscan FC system (four-parameter logistic algorithm was applied). Vitamin B12 concentrations in the original samples were obtained by re-calculation taking into account sample dilutions during material processing. Once all measurements were completed, the results of the two cooking experiments were combined by taking the average concentration value for each set of conditions.

### Example 17

Influence of Astaxanthin and Lycopene Added to Olive Oil on Vitamin D3 Loss from Food During Cooking.

The purpose of this work package was to assess possible loss of Vitamin D3 due to thermal processing applied during food preparation. The work has been done on the basis of one main experiment: determination of Vitamin D3 concentration in wild salmon cooked under different conditions.

Methodology

Wild pacific Keta salmon filets (app. 115 g each) were oven-cooked (oven temperature 180° C.) in individual small aluminium foil containers. The following preparation conditions were applied before cooking: 1) Addition of 25 ml of Napolina olive oil containing Astaxanthin (7 mg/30 ml); 2) Addition of 25 ml of Napolina olive oil containing Lycopene (7 mg/30 ml); 3) Addition of 25 ml of pure Napolina olive oil; 4) Addition of 25 ml of water. Salmon filets were turned several times making sure that they were completely covered with the added liquid following oil or water addition respectively. After that 500 mg of salt and 5 ml of fresh lemon juice were applied on the surface of each portion of fish. During the cooking process the internal (doneness) temperature of the fish was measured using a digital thermometer at the following time points: 8 minutes, 12 minutes, 16 minutes, and 20 minutes. All containers had to be taken out of the oven for temperature measurement, hence only time when the fish was in the oven was counted as cooking time. Once the internal temperature of the fish reached 62° C. (doneness temperature for salmon), a small (about 1 g) fragment of fish was immediately taken from the relevant piece (using a scalpel blade and thumb forceps) and placed in a 15 ml laboratory tube containing 1 ml of distilled water. An additional sample of fish was also taken before cooking. 1 ml samples of fish ‘juice’ from the pack of fresh salmon and ‘sauce produced when salmon was cooked with water were taken as well. Temperature measurements for each time point were registered.

Salmon Sample Preparation

Samples were transferred to the laboratory and weighed using analytical scales (Discovery DV114C, OHAUS Corp.). Distilled water was added to each sample apart from the ‘juice’ and ‘sauce’ samples to provide the ratio of 9 ml of water per 1 g of sample (these samples were regarded as 1/10 dilutions). 1 ml of distilled water was added to the ‘juice’ and ‘sauce’ samples to produce 1/2 dilutions. Following this step all samples were homogenised using IKA T10 basic Ultra-Turrax homogeniser system at maximum speed (30,000 RPM). After homogenisation of each sample the homogeniser was disassembled and both its rotor and stator were carefully cleaned in order to prevent sample cross-contamination. Following homogenisation second dilutions with four volumes of distilled water (1 ml homogenate+4 ml of water) were prepared from the homogenates of the raw fish, fish cooked with water, fish cooked with pure olive oil, fish cooked with olive oil containing Lycopene and fish cooked with olive oil containing Astaxanthin (resulting in 50× dilutions). Similar dilutions were made from salmon ‘juice’ and ‘sauce’ samples described above (resulting in 10× dilutions). Finally, additional five-fold dilutions were made from the 50× dilutions of homogenates prepared from salmon cooked with olive oil, olive oil containing Lycopene and olive oil containing Astaxanthine (resulting in 250× dilutions). Once all sample dilutions were prepared, the extraction procedure was performed mostly according to the recommendations provided in the instructions for VitaKit D™ (Chrystal Chem, Cat. No. 72051). It should, however, be noted that modifications had to be introduced into the extraction procedure since the kit is designed for milk sample analysis. 1 ml of each homogenate was taken into a new 15 ml laboratory tube. 0.55 g of granular KOH was added to each tube. The tubes (put into a rack) were covered with aluminium foil (in order to protect samples from light) and subjected to three cycles of 4-minute incubation followed by vigorous shaking for 2 minutes. Then 2 ml of hexane was added to each tube, tubes were closed, again covered with aluminium foil and vigorously shaken for 2 minutes. After that all tubes were centrifuged for 5 minutes at RCF=1920 g (4500 RPM) using Hettich Universal 320 laboratory centrifuge. 200 μl of the resulting supernatant from each tube was transferred into pre-labelled individual Eppendorf tubes for Vitamin D3 concentration determination and kept in the dark before analysis.

Vitamin D3 Concentration Determination

VitaKit D™ ELISA Kit (Chrystal Chem, Cat. No 72051) was used for Vitamin D3 concentration determination in extracts produced from sample homogenates (see above). D3 concentration determination was performed in 10 μl fractions of the extracts following evaporation according to the protocol provided with the kit. All incubation steps were performed in the dark as recommended by the protocol.

The necessity of performing the ELISA test immediately following Vitamin D3 extraction limited the maximal number of assays in the experiment. For this reason nine microtiter well strips were removed from the microtiter plate provided with the kit. Only three microtiter well strips were used in the experiment.

Vitamin D3 concentration determination was performed using Multiscan FC microplate photometer (Thermo Fisher Scientific) by measuring light absorbance at 450 nm. The results were analysed using Skanit software for Multiscan FC (linear regression algorithm was applied). Vitamin B12 concentrations in the original samples were obtained by re-calculation taking into account sample dilutions during material processing.

Results

Reduction of Cooking Time

In our experiments it was shown that addition of carotenoids to the cooking oil resulted in reduction of time needed for cooking.

For example, the chicken liver cooked 2 minutes and 12 seconds faster when only 1 molecule of lycopene was embedded into its 67,000 molecules of fatty acids of olive oil. When 1 molecule of astaxanthin was added to 70,000 of the fatty acids the cooking time accelerated by 5 minutes, or by one third in comparison with the control olive oil process, FIG. 35.

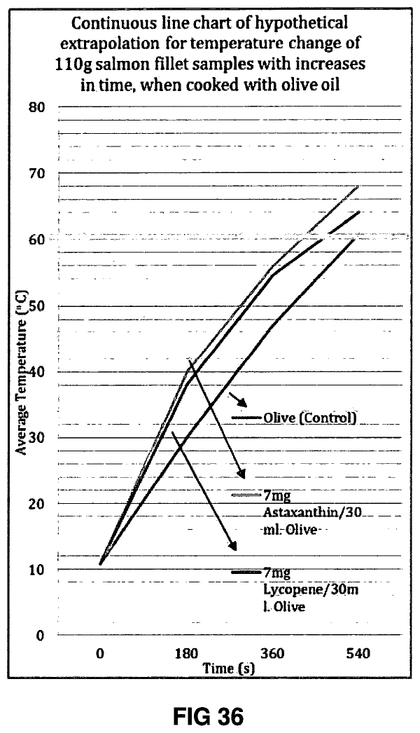

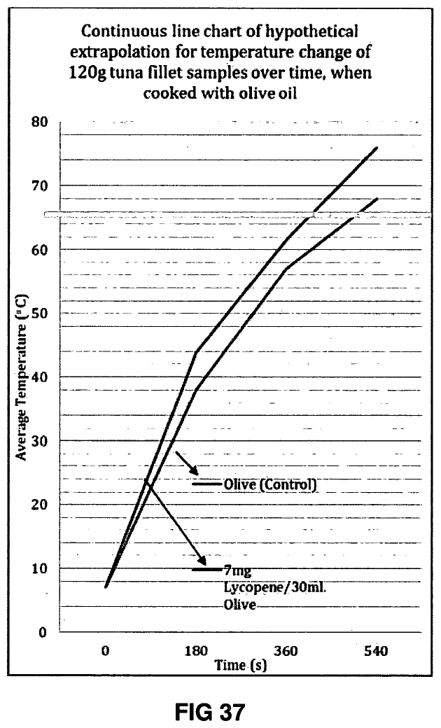

A similar positive effect was observed when fish was cooked. The cooking time in the lycopene olive oil was reduced by 41 seconds for the salmon fillet, and in astaxanthin oil by 1 minute and 4 seconds, FIG. 36. In another experiment cooking in the lycopene oil the tuna fillet was ready 1 minute faster, FIG. 37.

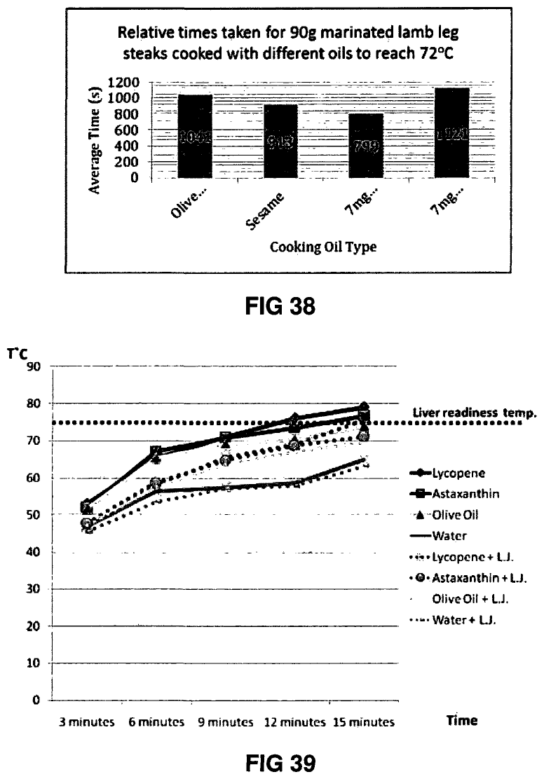

When lamb was cooked the astaxanthin oil did not have an accelerating effect as in above experiments. However, cooking in the lycopene oil the cooking time was reduced by 4 minutes and 2 seconds, or by one third in comparison with the control olive oil, FIG. 38.

Preservation of Health Important Nutrients, Vitamins D3 and B12

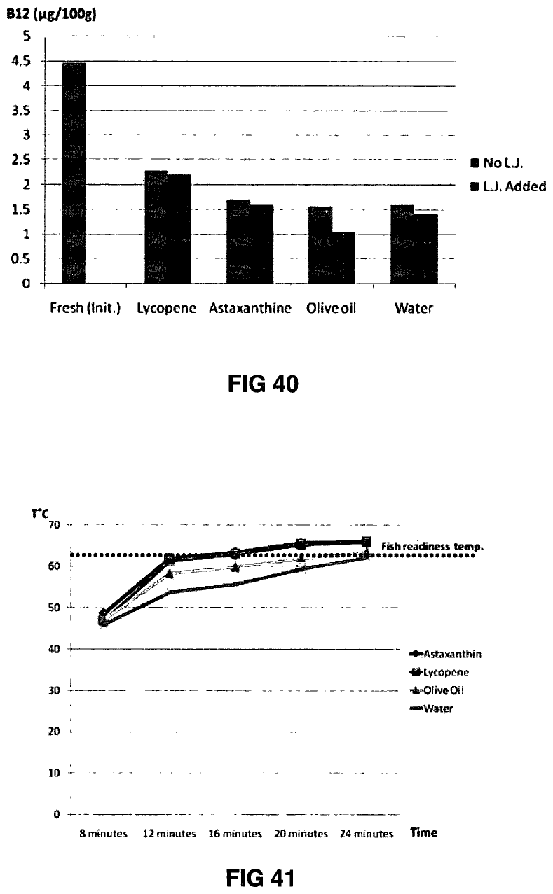

Acceleration of the cooking time for the chicken liver in the lycopene oil by more than 3 minutes and in the astaxanthin oil by 2 minutes, FIG. 39, resulted in a significantly higher level of preserved vitamin B12 in the ready to eat product. The concentration of this vitamin was more than double in the first experiment, regardless of whether it was cooked with or without additional lemon juice. Although the concentration of B12 was also higher for the astaxanthin oil, the difference was significant, by 50%, only when the liver was cooked with the lemon juice, FIG. 40.

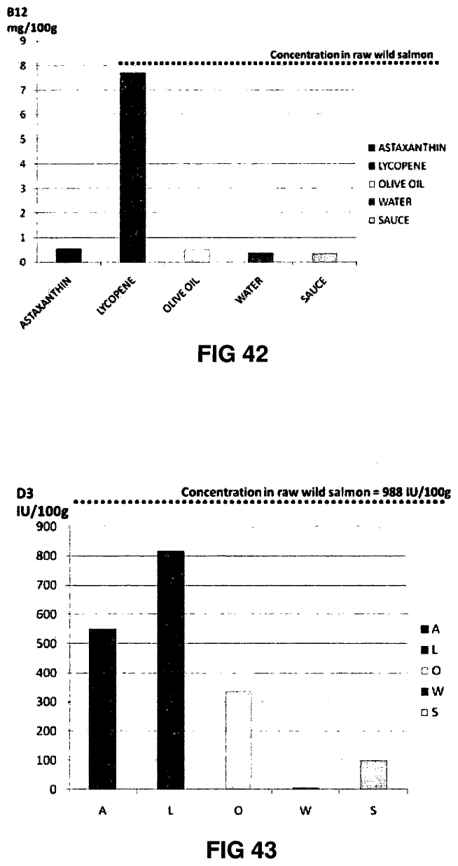

Acceleration of the cooking time for the wild salmon either in the lycopene or astaxanthin oil reduced it from 24 to 14 minutes, or by 40%, FIG. 41. However, it did not translate to any significant preservation of vitamin B12 in the product cooked in the latter experiment. At the same time cooking in the lycopene oil resulted that in the ready to eat fish the level of this vitamin was 15 fold higher than in the fish cooked in the control olive oil, FIG. 42.

For the vitamin D3 accelerated cooking, time was translated to its preservation for both types of carotenoid oils. For the astaxanthin oil, concentration of this vitamin was more than 60% higher than in the controlled cooked product, and for the lycopene oil it was about 2.4 times more, FIG. 43.

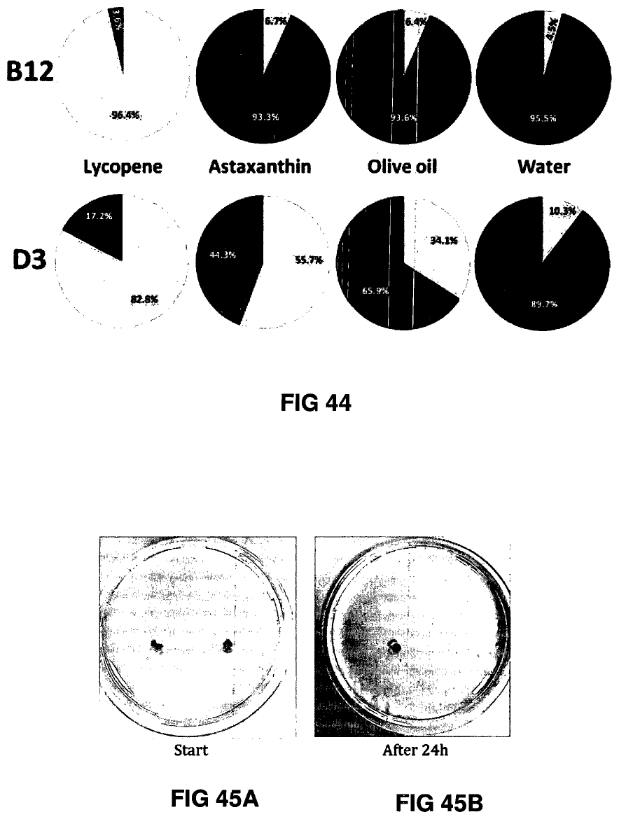

The summary of the preservation effect on vitamins B12 and D3 in the fish cooked in in oils with embedded carotenoids is presented in the FIG. 44.

### Conclusions

Increase of thermal conductivity and heat capacity of hydrocarbons, fats and oils in particular, indicates that the disruption of their folding by carotenoids could be used across broad range of products and applications. It is known that oils with higher thermal conductivity will transfer heat energy more effectively.

These properties can be used but are not limited:
to increase heat transfer abilities of engine, or radiator, or other industrial oils, or lubricants, or hydraulic or other fluids, which would result in reduction and mitigation of wear, and ultimately improvement of the efficacy of an engine or other machinery where they are used;
to increase heat transfer abilities of cooking oils with embedded carotenoids would lead to accelerating of the cooking or heating food processes, which in turn would result in saving time and fuel required for the cooking, hence increase the efficacy and reduce the cost of the process;
to preserve or reduce loss of essential nutrients, vitamins and other health important molecules in cooking with oil food, which can be degraded or neutralized by the heat; as a result of this—
to improve nutritional value of the cooked food.

Carotenoids Increase Fuel Efficacy of Hydrocarbons

The increase of their thermal conductivity and heat capacity of oils, fats and other hydrocarbons may in turn lead to increase in fuel efficacy.

### Example 18

Carotenoids Improve Efficacy of Burning Lamp Oil

Different dilutions of lycopene and astaxanthin were made of lamp oil, Paraffin (petroleum), C5-20, with flash point >65 C.

In this experiment two parameters were measured:
the burning time—the time from the lightening the wick and starting the flame, to its own extinguishing,
the residual amount of fuel left in the lamp after this extinguishing.

The experiment of the same design was repeated for different ranges of lamp oil volumes and carotenoid concentrations.

Results

In the experiment with 15 ml of the lamp oil in all lamps containing oil with carotenoids the flame was burning significantly longer than in the control lamp. When flame was extinguished itself in all lamps the residual volume of the fuel was measured. In all four lamps with astaxanthin oil and in three out of four with lycopene the residual volume of the oil was significantly larger than in the control lamp, between 2 and 5 fold, table 7.

TABLE 7
Residual volume of oil left in lamps after flame was extinguished

| | Astaxanthin in lamp | | | | Lycopene in lamp | | | |
|---|---|---|---|---|---|---|---|---|
| Control | oil, in μg/ml | | | | oil, in μg/ml | | | |
| oil | 2 | 4 | 8 | 16 | 2 | 4 | 8 | 16 |
| 2.2 ml | 4.7 ml | 4.5 ml | 9.5 ml | 10.5 ml | 9.0 ml | 2.0 ml | 11.2 ml | 5.0 ml |

In the experiment when the volume of the oil was increased to 33 ml, the burning time for the flame in the control lamp was about 7 hours, but in the lamp with astaxanthin oil the flame was observed after 8 hours. At the same time, like in the previous experiment, despite the fact that the burning of the flame in the lamp with oil containing this carotenoid lasted longer the amount of the burned oil was noticeably smaller. In this experiment the flame extinguished itself in 8 hours. The remaining un-burned volume of oils was 5 ml. When the person conducting this experiment extinguished the still burning oil with astaxanthin, the residual volume of oil in the lamp was 14 ml. This means 28 ml of oil was burned in 8 hours in the control experiment, i.e. the burning rate was 3.5 ml/hour.

In the astaxanthin oil 19 ml of oil was burned for the same time, hence the burning rate was 2.375 ml/hour, or almost 1.5 times more efficient.

In the following experiment when the volume of the lamp oil was increase to 100 ml the same effect was observed. The burning of the flame lasted more than 5 hours longer in the lamp oil with astaxanthin, but at the amount of the oil burned was significantly less than in the lamp with the control oil.

### Conclusions

The observed increase of the burning time of the same or even reduced amount of fuel is an indication that incorporation of carotenoids increases the fuel efficacy of oils and maybe other hydrocarbons. This use of carotenoids to disrupt the configuration, structural organisation, packing and interaction of the molecules of a hydrocarbon with each other could have a number of practical applications:
to increase burning time of fire, or flame, or heat generation without increase of, or with reduced, fuel consumption, hence
to increase efficiency of lamps, engines, generators, or other machinery or devices which burn fuel to produce light, heat, electricity, magnetism, mechanical or other forms of power, and consequently on the one hand to increase their efficacy and performance, and on another to save the cost of the fuel consumed and reduce the cost of using these devices.

Carotenoids Increase Size of Lipid Droplets and Globules and Reduce Viscosity of Edible Oils and Fats, which Results in:
Reduced Rate of Acidic/Stomach Degradation, hence Improve Lipids Bioavailability,
Reduced Lipid Enzymatic Digestion and Reduced Transit Time in Gastro-Intestinal Tract.

a) New Pharmaceuticals, Nutraceuticals and Functional Food with Increased Bioavailability and Improved Clinical Efficacy.

Stomach pH is a highly acidic environment and varies from 1.5 or 3.5 subject to its emptiness. Some hydrocarbons, and particular polyunsaturated fatty acids or phospholipids, which have nucleophilic elements in their structures, could be easily oxidised, modified and eventually degraded at this acidic pH. Since ingested lipids are presented in the stomach in a form of suspension or colloid, the main limiting factor regulating the rate of these particles oxidation will be the size of their surface.

### Example 19

Enlargement of Lipid Droplets by Carotenoids Protect them from Acidic Modification/Degradation.

This statement was confirmed in two in vitro experiments one with a fatty acid, DHA Omega 3, and another one with a phospholipid, Phosphatidylcholine PTC.

Method.

To measure and quantify these lipids we used Gas Chromatography analysis.

DHA and PTC concentrations in all samples were measured in duplicate by a gas-liquid chromatography (Bowen, Kehler, Evans 2010), with slight modifications. Briefly, 2 ml of stock solution required for each sample included 1.9 ml of methanol and 100 μl of acetyl chloride. Briefly, 100 μl of serum and 2 ml of the stock solution were combined in screw-capped glass tubes. The tubes were capped and heated at 100 C for 60 min. The tubes were allowed to cool down to the room temperature and were extracted twice with 1 ml hexane. The combined hexane solution was evaporated under vacuum (Scan Speed 32 centrifuge) and the residue reconstituted to the volume of 50 μl with of hexane, transferred to GC vials, and capped under nitrogen.

Fatty acids analyse was performed with a fused silica capillary column (HP-5), 30 m 0.32 mm inner diameter (ID), 0.25 m film thickness (Hewlett Packard, USA), a Shimadzu GC 2010 Gas chromatograph with Flame Ionization Detector and manual injection system (Shimadzu, Japan). Temperature program, initial: 130 C with a 4 min hold; ramp: 4 C/min to 280 C with a 2 min hold. Carrier gas was He, with a linear velocity of 30 cm/s. Fatty acid analysis was performed by injection of 1 μl of each sample at a split ratio of 50:1. The FID and the Injection port temperature was 300 C. The sampling frequency was 40 Hz. Fatty acids identification was performed with SUPELCO 37 COMPONENT FAME MIX (Supelco, USA). DHA concentrations were calculated on the basis of the standard concentrations. Analytical standard of DHA and PTC (Sigma, USA) were used in the assay.

To measure malondialdehyde, MDA, which is the end product of lipid acidic peroxidation we used earlier described method (Petyaev et al., 2012) when analysed samples were incubated overnight in 0.05 M PBS acetate buffer (pH 5.6). The following morning the reaction was stopped using trichloroacetic acid. The concentration of the MDA, and other possible thiobarbituric acid reactive substances (TBARS), was then measured by colorimetry using reagents and kits from Cayman Chemical (MC, USA).

Results.

Incubation of DHA in 0.05M phosphoric acid—sodium hydrochloride buffer pH2.5, at 37° C., resulted in a significant degradation of this fatty acid. However, when lycopene was blended in concentration of 1 mg or 3 mg per 1 gram of DHA this degradation was completely prevented, table 8a.

In this experiment we used the commercial preparation, which contained only 40% of DHA, the rest of the mass was a blend into other lipids. It was interesting to note that acidic oxidation was not only responsible for the degradation of DHA, but 19 other fatty acids, apart of pamitoleic acid, which were present in this product, and they include other omega 3 fatty acids—eicosapentaenoic acids, and also others too poly- and mono-unsaturated, saturated, short, medium and long chain fatty acids (table 8b).

TABLE 8a
Gas chromatography analysis of the changes in the
DHA matrix caused by Lycopene reduced its susceptibility
to acid induced degradation, 0.05M pH 2.5.

| Product composition | Incubation time, in min | DHA in μg/ml |
|---|---|---|
| 40% DHA | 0 | 987 |
| | 30 | 630 |
| | 60 | 665 |
| | 90 | 750 |
| | 120 | 636 |
| 1 mg lycopene per | 0 | 853 |
| 1 gram DHA | 30 | 1,048 |
| | 60 | 999 |
| | 90 | 807 |
| | 120 | 1,003 |
| 3 mg lycopene per | 0 | 887 |
| 1 gram DHA | 120 | 862 |

Incorporation of lycopene into DHA preparation resulted in the reduction of the susceptibility of not only this fatty acid, but all others present therein, to the acidic oxidation/degradation caused by incubation at pH2.5 for 2 hours, table 8c.

TABLE 8b
Incubation at pH 2.5 for 2 hours led to acidic oxidation/degradation
of DHA, and other fatty acids present in its preparation;
gas chromatography analysis.

| | | | | After 2 hours at | |
|---|---|---|---|---|---|
| | | Baseline | | pH 2.5 | |
| Peak | Fatty acids | Area | Area, % | Area | Area, % |
| 1 | Caproic | 13,975 | 1.31 | n/d | n/d |
| 2 | Lauric | 1,718 | 0.16 | n/d | n/d |
| 3 | Myristoleic | 1,934 | 0.18 | 1,197 | 0.18 |
| 4 | Myristic | 73,096 | 6.23 | 43,118 | 6.42 |
| 5 | Pentadecanoic | 3,120 | 0.29 | 1,716 | 0.26 |
| 6 | Palmitoleic | 1,219 | 0.11 | 1,278 | 0.19 |
| 7 | Palmitic | 197,526 | 18.45 | 118,649 | 17.66 |
| 8 | gamma-Linolenic | 3,030 | 0.28 | 1,851 | 0.28 |
| 10 | Linoleic | 9,494 | 0.89 | 8396 | 1.25 |
| 11 | Linole/alpha- | 69,917 | 6.53 | 45112 | 6.72 |
| | linilenic | | | | |
| 12 | Elaidic | 2,108 | 0.20 | 1629 | 0.24 |
| 13 | Stearic | 7,051 | 0.66 | 4597 | 0.68 |
| 14 | Arachidonic | 8,907 | 0.83 | 5323 | 0.79 |
| 15 | cis-5,8,11,14,17- | 11,447 | 1.07 | 6679 | 0.99 |
| | eicosapentaenoic | | | | |
| 16 | cis-8,11,14,17- | 5,002 | 0.47 | 2800 | 0.42 |
| | eicosapentaenoic | | | | |
| 18 | Arachidic | 1,032 | 0.10 | n/d | n/d |
| 20 | cis-4,7,10,13,16,19- | 456,572 | 42.63 | 292,485 | 43.54 |
| | Docosahexaenoic | | | | |
| 22 | Behenic | 1,096 | 0.10 | n/d | n/d |
| 23 | Lignoceric | 1,518 | 0.14 | 1162 | 0.17 |
| Total | | 869,762 | 81.22 | 535,992 | 79.80 |

To confirm that the enlargement of lipid droplets, caused by incorporating of lycopene into their matrix, may not only reduce the degradation of their intact structures, as we observed in the above experiments, but also resulted in inhibitor of the whole oxidative cascade caused by acidic degradation, which we analysed by measuring the end products of this cascade, MDA groups.

TABLE 8c
Lycopene changes in folding of DHA, and other fatty
acids, reduced their susceptibility to the acidic
oxidation/degradation; lycopene concentration was
1 mg per 1 g of DHA; gas chromatography analysis.

| | | | | After 2 hours at | |
|---|---|---|---|---|---|
| | | Baseline | | pH 2.5 | |
| Peak | Fatty acids | Area | Area, % | Area | Area, % |
| 1 | Lauric | 1,354 | 0.14 | 1,605 | 0.15 |
| 2 | Myristoleic | 1,662 | 0.17 | 1,789 | 0.17 |
| 3 | Myristic | 61,814 | 6.42 | 69,816 | 6.56 |
| 4 | Pentadecanoic | 2,511 | 0.26 | 2,843 | 0.27 |
| 7 | Palmitoleic | 1,598 | 0.17 | 1,710 | 0.16 |
| 9 | Palmitic | 171,664 | 17.82 | 186,438 | 17.52 |
| 10 | gamma-Linolenic | 2,627 | 0.27 | 2,767 | 0.26 |
| 12 | Linoleic | 12,328 | 1.28 | 10,395 | 0.98 |
| 13 | Linole/alpha- | 63,846 | 6.63 | 68,330 | 6.42 |
| | linilenic | | | | |
| 14 | Elaidic | 2,537 | 0.26 | 2,205 | 0.21 |
| 15 | Stearic | 6,673 | 0.69 | 6,811 | 0.64 |
| 16 | Arachidonic | 7,859 | 0.82 | 8,565 | 0.81 |
| 17 | cis-5,8,11,14,17- | 9,937 | 1.03 | 10,759 | 1.01 |
| | eicosapentaenoic | | | | |
| 18 | cis-8,11,14,17- | 4,391 | 0.46 | 5,889 | 0.55 |
| | eicosapentaenoic | | | | |
| 23 | cis-4,7,10,13,16,19- | 409,974 | 42.55 | 464,051 | 43.60 |
| | Docosahexaenoic | | | | |
| 29 | Lignoceric | 1,613 | 0.17 | 1,775 | 0.17 |
| Total | | 762,388 | 79.13 | 846,908 | 79.58 |

Incubation of suspensions of preparations of DHA and Phosphatidylcholine in the same, as in the experiment above, 0.05M phosphoric acid buffer with pH2.5 for 2 hours resulted in a time-dependent accumulation of MDA. Blended into these lipids lycopene resulted in a significant reduction of this parameter, which indicates that this carotenoids changed lipids and made them less sensitive to the acidic oxidation (table 9).

TABLE 9
Lycopene changes in folding of PTC and DHA resulted in
their reduced susceptibility to the acidic oxidation/degradation;
all experiments were made in duplicates.

| | Incremental changes in MDA concentration | | | | |
|---|---|---|---|---|---|
| | at different incubation time, in μM/ml | | | | |
| Products | 0 min | 30 min | 60 min | 90 min | 120 min |
| 75% PTC control | 0 | 0 | 5.5 | 97 | 302.5 |
| 1 mg lycopene per | 0 | 9 | 14 | 12.5 | 85.5 |
| 1 g PTC | | | | | |
| 3 mg lycopene per | 0 | 16 | 34 | 1.5 | 0 |
| 1 g PTC | | | | | |
| 5 mg lycopene per | 0 | 11.5 | 0 | 0 | 0 |
| 1 g PTC | | | | | |
| 40% DHA control | 0 | 17.5 | 141.5 | 169.5 | 553 |
| 1 mg lycopene per | 0 | n/a* | n/a* | n/a | n/a |
| 1 g DHA | | | | | |
| 3 mg lycopene per | 0 | n/a* | n/a* | n/a* | 364 |
| 1 g DHA | | | | | |
| 5 mg lycopene per | 0 | 58.5 | 70 | 215 | 250.5 |
| 1 g DHA | | | | | |

*development of products absorption of which was overlapping and not related to MDA.

### Example 20

Ingestion of Improved by Carotenoids Acid-Resistant Lipid (Droplets) Resulted in a Significant Increase in their Bioavailability and Clinical Efficacy.

In the experiments above we demonstrated that carotenoids can significantly increase the size and the surface of lipid droplets/formations, including at the pH2.5 (FIGS. 6c and d), which results in a significant protection of lipid molecules from their acidic oxidation/degradation. This may result that a higher level of these unmodified and active molecules be present in the gastrointestinal tract and be absorbed. The increase in their bioavailability could consequently be translated into higher clinical efficacy of these lipids.

Clinical Trial DHA-Lycopene.

To validate this, a clinical trial was undertaken. 32 individuals aged 40-65 years old with a moderate hyperlipidemia (serum triglycerides, TG, >150 mg/dl and LDL from 130 to 160 mg/dl). They were randomised and split in four groups of 8:
The first was supplemented with a 250 mg of conventional formulation of DHA without lycopene (CF-DHA),
the second with 7 mg of lycopene,
the third with 250 mg of DHA and 7 mg of lycopene co-ingested at the same time but as two separate products, and
the fourth group received 7 mg lycopene formulated with 250 mg of DHA (LF-DHA).

All products were taken once a day with the main meal. The duration of the trial was for 4 weeks.

It was shown that ingestion of CF-DHA led to a significant, 19.5 mg/dl, p<0.05, reduction in LDL, which was accompanied by a corresponding decrease in total serum cholesterol, and a much stronger reduction in serum TG concentration, reduction of medians by 27.5 mg/dl (table 10).

TABLE 10
Comparative pharmacodynamics of DHA and DHA-Lycopene on elevated serum
LDL and triglycerides in 4-week trial (Medians with 95/5%% Cis)

| | LDL in mg/dL | | | | Triglycerides in mg/dL | | | |
|---|---|---|---|---|---|---|---|---|
| Time | | | Co- | DHA- | | | Co- | DHA- |
| point | DHA | Lycopene | Ingestion | Lycopene | DHA | Lycopene | Ingestion | Lycopene |
| 0 w | 155.5 | 156.0 | 155.5 | 153 | 191.5 | 203.5 | 199.0 | 194.5 |
| | 150/158 | 150/160 | 152/159 | 150/158 | 180/201 | 191/211 | 187/207 | 182/207 |
| 4 w | 153.5 | 155.5 | 151.5 | 133.5* | 185.5 | 190.0 | 189.5 | 167.0* |
| | 148/157 | 149/159 | 147/159 | 123/138 | 180/201 | 182/199 | 186/200 | 160/187 |

Since the main mode of action of DHA Omega 3 is the inhibition of triglyceride synthesis in the liver, the efficacy of these absorbed molecules could be assessed by their ability to affect this parameter in the blood. The clinical trial results demonstrated that the blend of DHA-Lycopene was 4.5 fold stronger to reduce elevated TG than the same dose of DHA alone. This would allow us to assume that the increase of the surface of DHA droplets caused by lycopene, which may result in their higher tolerance to the stomach/acidic degradation and better preservation of the fatty acid molecules in this environment, could be translated into higher bioavailability and ultimately efficacy of this DHA.

Clinical Trial PTC-Lycopene.

29 newly diagnosed individuals with Non-Alcoholic Fatty Liver Disease (NAFLD) remaining on habitual dietary regimen were supplemented orally on a daily basis either with 450 mg of regular formulations of phosphatidylcholine (PTC), or with a formulation containing 450 mg PTC with 7 mg of blended in Lycopene. The participants in both groups were randomised, and it was a double blind clinical study. After two months of this treatment the liver size, as well as serum levels of hepatic enzymes and markers of inflammation were evaluated by ultrasonography and biochemical analysis. It was shown that there was a statistically significant reduction of medians for the Mid-Clavicular liver size from 16.0 cm (95/5% CI: 17.1/15.5) to 15.1 cm (95/5% CI: 17.2/14.4, P=0.021) in participants ingesting the lycopene-formulated PTC (L-PTC) whereas regular formulation of PTC (R-PTC) had only a marginal effect on this parameter (P=0.044). A similar tendency was observed in the Mid-Sternal liver size (table 11a). Moreover, there was a reduction of medians for ALT values at the end point of the study (P=0.026) after ingestion of L-PTC, while R-PTC had no statistically significant effect (table 11b). On the other hand, ingestion of both formulations was accompanied by reductions in values for Inflammatory Oxidative Damage (IOD) and oxidized LDL in serum. C-reactive protein level was moderately decreased (reduction of medians from 6.5 [95/5% CI: 7.7/5.8] mg/L to 5.1 [95/5% CI: 5.6/4.3] mg/L) only after ingestion of L-PTC. The greater efficacy of L-PTC seen in NAFLD volunteers may reflect improved bioavailability of PTC owing to better protection of the enlarged droplets of PTC by lycopene from stomach acidity and possibly other gastro-intestinal factors, which may affect PTC integrity and ultimately its clinical efficacy.

TABLE 11a
Liver span parameters after treatment with R-PTC and L-PTC
(Medians with 95/5%% CI)

| DURATION OF | FORMULATIONS OF PTC | |
|---|---|---|
| TREATMENT | Regular PTC | Lycopene-PTC |
| | Mid-Clavicular Size (cm) | |
| Baseline | 16.1 (16.8/15.1) | 16.0 (17.1/15.5) |
| 1 Month | 16.0(16.8/14.7) | 15.7(17.2/15.0) |
| 2 Months | 15.6(16.4/14.5)* | 15.1(17.2/14.4)* |
| | Mid-Sternal Size (cm) | |
| Baseline | 7.2(7.7/6.5) | 7.2(7.5/6.9) |
| 1 month | 7.1(7.57/6.2) | 7.0(7.5/6.2) |
| 2 Months | 7.0(7.3/6.2)* | 6.8 (7.2/6.5)* |

TABLE 11b
Serum AST and ALT after treatment with R-PTC and L-PTC
(Medians with 95/5%% CI)

| | |
|---|---|
| DURATION OF | FORMULATIONS OF PTC |

| | | |
|---|---|---|
| TREATMENT | Regular | Lycosome |

AST (IU/ml)

| | | |
|---|---|---|
| Baseline | 38.5 (45.0/34.6) | 38.0 (43.0/35.0) |
| 1 Month | 40.5(44.3/36.0) | 37.5(42.4/35.0) |
| 2 Months | 39.0(43.0/36.0) | 36.0(38.7/35.0) |

ALT (IU/ml)

| | | |
|---|---|---|
| Baseline | 61.0(67.3/52.9) | 59.5(68.7/54.0) |
| 1 month | 59.0(65.3/51.6) | 57.0(66.4/51.2) |
| 2 Months | 58.0(63.3/50.6) | 53.5 (60.0/47.6)* |

*for the both tables - P < 0.05 as compared to baseline

b) New Food with Reduced Lipid Digestibility and Postprandial Lipidaemia

Disruption of lipid folding by carotenoids could lead, as it was seen in the previous experiments, to increase size of lipid droplets or fat globules. It is a basic physiological fact that the larger lipid particles are the longer time is needed for stomach and pancreatic lipases, released in duodenum, to break them down.

Therefore when a person ingests a lipid-based product, droplets or globules of which are increased by carotenoids, this would lead to a reduced rate of this product digestion. As a result of this, especially when the ingested amount of the product is significant, there maybe not enough time for all its lipid molecules to be enzymatically processed and subsequently absorbed.

### Example 21

Since the main limiting factor affecting rate of enzymatic digestion of lipids is the size of the their emulsion, colloid or other particles, we undertook the following experiment to verify whether carotenoid enlargement of fat/oil droplets or globules may be translated to the reduction of their digestibility by a pancreatic lipase. In brief, two similar size samples of solid cocoa butter, with or without blended-in lycopene were place in a Petri dish on the surface of PBS, which contained dissolved porcine pancreatic lipase Type II (Sigma) in concentration 100 units per 1 ml. After incubation of the samples at 37° C. for 24 hours it was found that the control sample was apparently fully digested while a significant part of the cocoa butter with lycopene was not (FIG. 45 A-B).

In addition, the changes in configuration, structural organisation, packing and interaction of the lipids caused by formation of their complexes with carotenoids, as it was seen in the described above experiments, resulted in reduction of the viscosity of edible oils and fats. This would accelerate their passage through the gastro-intestinal tract, GIT, and consequently reduce exposure of these lipids to stomach and pancreatic lipases, to bile and other digestive factors.

Therefore a combination of two changes in the configuration, structural organisation, packing and interaction of the molecules of a with each other, increase of the diameter of their droplets and acceleration of their transit time in the GIT, would lead to slower rate of their enzymatic digestion. This will reduce the amount of lipids, which can be absorbed and enter into the circulating blood. This also means that carotenoid modification of edible oils and fats would result in food products with reduced “digestible” calories, or reduced body calories intake.

### Example 22

Formation of Lipid—Carotenoid Complexes with Changes in Configuration, Structural Organisation, Packing and Interaction of Lipid Molecules with Each Other, Leads to a Reduction of Lipid Absorption and Postprandial Lipidaemia.

Four crossover clinical trials were undertaken to compare postprandial lipidaemia after ingestion of food products comprised either in full or in part of fat, and made without or with embedded lycopene. Full fat products were sunflower oil and dairy butter. One product containing about 30% of fat was dark chocolate, and the fourth product containing only about 15% of fat was ice cream.

Results

In the first crossover study the volunteers were asked to ingest 50 g of intact sunflower oil with one slice of white bread. The blood was taken before the experiment and every hour for 4 hours after its beginning. The same experiment was repeated a week later but this time the same volunteers were asked to ingest the same amount of this oil embedded with 30 mg of lycopene.

Results of this crossover study are presented in a double table 12a-b.

The mean of the Area Under the Curve, AUC, after ingestion of the control sunflower oil was for total cholesterol 56.4±5.9 mg/dL, for LDL-cholesterol 17±1.9 mg/dL and for triglycerides 25.5±2.8 mg/dL. When the same amount of oil was ingested but with embedded lycopene the mean AUC in the postprandial serum was significantly lower for two out of three lipid parameters, for the total cholesterol it was 32±3.5 p<0.01, for LDL cholesterol 14±1.6 p>0,05, and for triglycerides 11.5±1.6 p<0.01.

TABLE 12a
Postprandial lipidaemia after ingestion of control sunflower oil.

| | | | | | Δ = AUC 1-4 hours | | |
|---|---|---|---|---|---|---|---|
| 50 g Sunflower Oil Control | | | | | TC | TG | LDL |
| ID | Gender | Age | Height | Weight | mg/dL | mg/dL | mg/dL |
| VAK | m | 66 | 175 | 83 | 7 | 8 | 4 |
| IYR | f | 55 | 163 | 69 | 14 | 14 | 4 |
| NEC | f | 59 | 168 | 76 | 105 | 5 | 20 |
| INK | f | 66 | 161 | 72 | 141 | 93 | 23 |
| RYC | m | 33 | 180 | 78 | 40 | 31 | 8 |
| VYK | m | 59 | 172 | 85 | 29 | 0 | 45 |
| IOL | f | 61 | 162 | 67 | 33 | 18 | 9 |
| AAA | m | 37 | 165 | 73 | 82 | 35 | 23 |

TABLE 12b
Postprandial lipidaemia in volunteers after ingestion
of sunflower oil with embedded 30 mg of lycopene.

| | | | | | Δ = AUC 1-4 hours | | |
|---|---|---|---|---|---|---|---|
| 50 g Sunflower with 30 mg lycopene | | | | | TC | TG | LDL |
| ID | Gender | Age | Height | Weight | mg/dL | mg/dL | mg/dL |
| VAK | m | 66 | 175 | 83 | 10 | 1 | 13 |
| IYR | f | 55 | 163 | 69 | 19 | 22 | 17 |
| NEC | f | 59 | 168 | 76 | 68 | 0 | 16 |
| INK | f | 66 | 161 | 72 | 63 | 2 | 10 |
| RYC | m | 33 | 180 | 78 | 42 | 18 | 14 |
| VYK | m | 59 | 172 | 85 | 33 | 15 | 22 |
| IOL | f | 61 | 162 | 67 | 6 | 11 | 8 |
| AAA | m | 37 | 165 | 73 | 15 | 23 | 12 |

In the second crossover experiment it was shown that ingestion of 50 grams of dairy butter with embedded 30 mg lycopene accompanied significantly lower AUC for all three measured lipid parameters than after ingestion of the control butter samples, table 13a-b.

TABLE 13a
Postprandial lipidaemia after ingestion of control dairy butter.

| | | | | | Δ = AUC 1-4 hours | | |
|---|---|---|---|---|---|---|---|
| 50 g butter control | | | | | TC | TG | LDL |
| ID | Gender | Age | Height | Weight | mg/dL | mg/dL | mg/dL |
| VAK | m | 73 | 175 | 82 | 24 | 15 | 2 |
| TOL | m | 22 | 181 | 73 | 53 | 21 | 3 |
| FTA | f | 59 | 164 | 81 | 65 | 24 | 12 |
| KRD | f | 42 | 161 | 77 | 19 | 24 | 1 |
| NEC | m | 31 | 178 | 70 | 5 | 21 | 1 |
| REC | m | 45 | 169 | 85 | 20 | 26 | 4 |
| POR | f | 62 | 152 | 94 | 50 | 45 | 2 |
| AZR | f | 57 | 161 | 76 | 30 | 21 | 3 |

| | | | |
|---|---|---|---|
| mean: | 33.3 | 24.6 | 3.5 |

TABLE 13b
Postprandial lipidaemia after ingestion
of dairy butter with embedded lycopene.

| | | | | | Δ = AUC 1-4 hours | | |
|---|---|---|---|---|---|---|---|
| 50 g Butter with 30 mg lycopene | | | | | TC | TG | LDL |
| ID | Gender | Age | Height | Weight | mg/dL | mg/dL | mg/dL |
| VAK | m | 73 | 175 | 82 | 19 | 16 | 1 |
| TOL | m | 22 | 181 | 73 | 35 | 11 | 1 |
| FTA | f | 59 | 164 | 81 | 44 | 21 | 5 |
| KRD | f | 42 | 161 | 77 | 12 | 19 | 1 |
| NEC | m | 31 | 178 | 70 | 5 | 10 | 1 |
| REC | m | 45 | 169 | 85 | 11 | 24 | 3 |
| POR | f | 62 | 152 | 94 | 23 | 31 | 1 |
| AZR | f | 57 | 161 | 76 | 25 | 22 | 2 |

| | | | |
|---|---|---|---|
| mean: | 21.7 | 19.3 | 1.9 |

In the third crossover study volunteers were asked first to ingest 100 g of dark chocolate, 70% cocoa. Then their blood was taken every hour for three consecutive hours. After one week rest the experiment was repeated with the same chocolate, but this time lycopene was embedded into it.

Results of the experiment are presented in tables 14a-b and 15a-b. They show that the mean of the postprandial AUC for three measured lipids after ingestion of the lycopene chocolate was lower than in the control experiment. Moreover, the mean of AUC for serum scattering, which measures total size of the pool of freshly absorbed lipids, was a half of the value in the former than in the latter experiment. It was interesting to note that in dark chocolate fat comprises only about 30% of it. Therefore, observed results indicate that cacao lipid modification caused by lycopene remains “undiluted” by the rest of the chocolate matrix.

TABLE 14a
Postprandial lipidaemia after ingestion of control dark chocolate

| | | | | | Δ = AUC 1-3 hours | | |
|---|---|---|---|---|---|---|---|
| 100 g dark chocolate control | | | | | TC | TG | LDL |
| ID | Gender | Age | Height | Weight | mg/dL | mg/dL | mg/dL |
| LN | m | 23 | 176 | 69 | 0 | 4 | 1 |
| LC | m | 27 | 171 | 58.5 | 23 | 64 | 2 |
| WG | m | 33 | 182 | 82 | 5 | 13 | 0 |
| LD | f | 57 | 165 | 82 | 6 | 2 | 3 |
| GW | m | 58 | 174 | 119 | 12 | 2 | 0 |
| SM | f | 58 | 189 | 87 | 35 | 8 | 4 |
| UR | f | 40 | 156 | 100 | 9 | 16 | 3 |
| GB | f | 59 | 164 | 114 | 23 | 6 | 5 |
| BR | m | 61 | 171 | 85 | 3 | 9 | 3 |
| GJ | f | 61 | 153 | 72 | 14 | 6 | 1 |
| GR | m | 63 | 168 | 82 | 7 | 1 | 0 |

| | | | |
|---|---|---|---|
| mean: | 12.5 | 11.9 | 2 |

TABLE 14b
Postprandial chylomicron scattering after
ingestion of control dark chocolate
Chylomicron Light Scattering

| Increment in nephelometric units, Δ LSI | | | |
|---|---|---|---|
| 1 h | 2 h | 3 h | AUC 1-3 hours |
| 51 | 36 | 87 | 174 |
| 159 | 89 | 248 | 496 |
| 53 | 282 | 335 | 670 |
| 123 | 2 | 125 | 250 |
| 60 | 126 | 186 | 372 |
| 99 | 178 | 277 | 554 |
| 60 | 19 | 47 | 126 |
| 36 | 61 | 23 | 120 |
| 41 | 82 | 23 | 146 |
| 33 | 87 | 13 | 133 |
| 12 | 73 | 19 | 104 |
| 66.1 | 94.1 | 126 | 286 |

TABLE 15a
Postprandial lipidaemia after ingestion
of dark chocolate with embedded lycopene.

| | | | | | Δ = AUC 1-3 hours | | |
|---|---|---|---|---|---|---|---|
| 100 g Dark Chocolate with 30 mg lycopene | | | | | TC | TG | LDL |
| ID | Gender | Age | Height | Weight | mg/dL | mg/dL | mg/dL |
| LN | m | 23 | 176 | 69 | 0 | 7 | 1 |
| LC | m | 27 | 171 | 58.5 | 13 | 12 | 1 |
| WG | m | 33 | 182 | 82 | 2 | 5 | 0 |
| LD | f | 57 | 165 | 82 | 10 | 8 | 3 |
| GW | m | 58 | 174 | 119 | 26 | 4 | 0 |
| SM | f | 58 | 189 | 87 | 6 | 7 | 3 |
| UR | f | 40 | 156 | 100 | 5 | 14 | 2 |
| GB | f | 59 | 164 | 114 | 12 | 9 | 3 |
| BR | m | 61 | 171 | 85 | 9 | 20 | 1 |
| GJ | f | 61 | 153 | 72 | 15 | 0 | 2 |
| GR | m | 63 | 168 | 82 | 5 | 4 | 0 |

| | | | |
|---|---|---|---|
| mean: | 9.4 | 8.2 | 1.45 |

TABLE 15b
Postprandial chylomicron scattering after ingestion
of dark chocolate with embedded lycopene.
Chylomicron Light Scattering

| Increment in nephelometric units, Δ LSI | | | |
|---|---|---|---|
| 1 h | 2 h | 3 h | AUC 1-3 hours |
| 71 | −4 | 67 | 134 |
| 0 | 118 | 118 | 236 |
| 10 | 84 | 94 | 188 |
| 25 | 77 | 102 | 204 |
| 7 | 93 | 100 | 200 |
| 50 | 44 | 94 | 188 |
| 25 | 9 | 11 | 45 |
| 19 | 21 | 8 | 48 |
| 33 | 52 | 17 | 102 |
| 27 | 76 | 3 | 106 |
| 15 | 38 | 12 | 65 |
| 25.6 | 55.3 | 56.9 | 137.8 |

In the fourth crossover study volunteers were asked first to ingest 100 g of control vanilla ice cream. Then their blood was taken every hour for three consecutive hours. After one week of rest the experiment was repeated with the same ice cram, and with the same its amount, but this time lycopene was embedded into its fat fraction.

Results of the experiment are presented in tables 16a and 16b. They show that after ingestion of the lycopene ice cream the postprandial AUC was lower than in the control experiment not only for two main lipids, total cholesterol and triglycerides, but for glucose too.

It was interesting to note that in the ice cream used it only comprised about 15% of. Therefore, observed results indicate that dairy fat lipid droplet modification caused by lycopene remains “undiluted” by the rest of the ice cream mass.

TABLE 16a
Postprandial lipidaemia after ingestion of control ice cream

| | | | | | Δ = AUC 1-3 hours | | |
|---|---|---|---|---|---|---|---|
| 100 g vanilla ice cream control | | | | | TC | TG | Glucose |
| ID | Gender | Age | Height | Weight | mg/dL | mg/dL | mm/L |
| VK | m | 66 | 172 | 92 | 1.2 | 1.8 | 0.9 |
| IR | m | 49 | 174 | 85 | 5.3 | 3.0 | 1.2 |
| IY | f | 33 | 165 | 74 | 1.2 | 0.2 | 1.3 |
| BG | f | 62 | 161 | 69 | 1.0 | 0 | 2.1 |
| GW | m | 45 | 168 | 101 | 9.0 | 0.9 | 1.7 |
| FT | f | 59 | 159 | 82 | 0.8 | 0 | 1.4 |

| | | | |
|---|---|---|---|
| mean: | 3.0 | 0.75 | 1.4 |

TABLE 16b
Postprandial lipidaemia after ingestion
of ice cream with embedded lycopene

| | | | | | Δ = AUC 1-3 hours | | |
|---|---|---|---|---|---|---|---|
| 100 g vanilla ice cream control | | | | | TC | TG | Glucose |
| ID | Gender | Age | Height | Weight | mg/dL | mg/dL | mm/L |
| VK | m | 66 | 172 | 92 | 0 | 0 | 0.4 |
| IR | m | 49 | 174 | 85 | 0 | 0 | 0.9 |
| IY | f | 33 | 165 | 74 | 0 | 0 | 1.1 |
| BG | f | 62 | 161 | 69 | 0 | 0 | 0.8 |
| GW | m | 45 | 168 | 101 | 0 | 0 | 0.2 |
| FT | f | 59 | 159 | 82 | 0 | 0 | 0.4 |

| | | | |
|---|---|---|---|
| mean: | 0 | 0 | 0.6 |

### Conclusion

These results indicate that formation of physical complexes between food lipids and incorporated into them carotenoids, can increase the size of their droplets or globules and reduce their transit time in the GIT, which consequently reduces the rate of digestion of these structures and ultimately reduce the level of lipid absorption.

This effect can be observed not only for full fat products but also when fat is only a part, or even a smaller part, of the food matrix.

Therefore carotenoids can be used to make oil or fat based, or oil or fat containing food products with reduced level of lipid digestion and lipid absorption, hence food with reduced calories not ingestion but intake.

Carotenoids Increase Size and Molecular Oxygen Capacity, and Reduce Viscosity of Lipid and Sebum Droplets, and Serum Lipoproteins: Boost of Mitochondria and Tissue Oxygenation

Introduction of carotenoids can create physical complexes with lipids, which would result in changes of the configuration, structural organisation, packing and interaction of lipid molecules with each other both in vitro and in vivo. This may lead to an increase in size of lipid structures and their hydrophobicity, hence increase the structural capacity to transport, supply and to retain molecular oxygen.

This can also reduce viscosity of lipid-rich adipose or other tissues and also sebum, impairment of which may occur in ageing or in development of a number of pathological conditions like obesity or acne.

### Example 23

In Vitro Studies

Carotenoids Stimulate Formation of Lipid Droplets, which Results in Boost of Mitochondria Growth and Respiration.

Reagents

Lycopene was kept in oxygen-free containers at −80° C. until used in the experiments. Stock oil solutions of lycopene (15%) were prepared using olive oil and kept at −20° C. For studies in cultured cells the 15% lycopene in oil stock solution was dissolved in DMSO at concentrations of 0.75, 1.5 and 3.0 mg/ml. Water dispersible microencapsulated lycopene as 10% suspension was mixed with DMEM to give a final concentration of 5 mg/ml.

Cell Lines

B10.MLM, a cell line of alveolar macrophages, was obtained from Prof. AS Apt (Institute of Tuberculosis, Moscow, Russian Federation). McCoy cells were obtained from the European Collection of Cell Cultures (Salisbury, UK). Cells were grown in 5% CO2 in DMEM supplemented with 2 mM glutamine and 10% FCS.

Experiment Design

Cells were grown in 24 well plates until a confluence rate of 80% was reached. After 1 hour of incubation the cell monolayers were washed with DMEM and lycopene additions were made. Oil solutions of lycopene diluted with DMSO were tested at final lycopene concentrations of 0.75, 1.5 and 3.0 μg/mL in the medium. Lycopene microencapsulated in dextran was added in medium up to final lycopene concentrations of 0.125, 0.25 and 0.5 mg/ml of DMEM. Control cells received additions of solvent or microencapsulation material (DMSO, olive oil or cyclodextrin) as single ingredients.

Lycopene Toxicity Verification

Lycopene toxicity was controlled in MTT test for 24 hours after lycopene addition using 96 well plates.

Neutral Lipid Staining

For neutral lipid staining B10.MLM cells grown on coverslips were incubated with lycopene for 24, 30 and 42 hours. Cells were then washed twice with PBS, fixed with 3% formaldehyde/0.025% glutaraldehyde at room temperature for 20 mins and stained with BODIPY 493/503 (Molecular Probes, Invitrogen Life Technologies, Carlsbad, Calif., USA) according to manufacturer's instructions. Cells were visualized using a Nikon Eclipse 50i fluorescence microscope at ×1000 magnification.

Automatic Image Processing Method for the Quantitative Analysis of Lipid Droplets

To improve the objectivity and reproducibility of the image assessment, we developed in-house automatic immunofluorescent image processing software that allows the reception of quantitative data on intracellular lipid droplets (10). The software measures the lipid droplet area in each cell from digital images of cell cultures. To perform automatic quantification we collected photos of 20 random fields of each sample. All images were uploaded into the program, and the size of lipid droplet area in cells was automatically evaluated.

Transmission Electron Microscopy (TEM)

B10.MLM cells were cultured with or without lycopene for 48 hours and then harvested from the plates with trypsin-versene solution. Cell pellets obtained by centrifugation for 10 min at 1500 r.p.m. (Rotanta 460R; Hettich) were fixed with Ito-Karnovsky fixative solution, followed by post-fixation with OsO₄ and treatment with aqueous uranyl acetate to provide contrast. The specimens were subsequently dehydrated in an ascending series of alcohol concentrations (50, 70, 96 and 100% ethanol), infiltrated with a 1:1 (v/v) mixture of LR White resin and 100% ethanol for 1 h and in a pure resin for 12 h at 4° C. Resin polymerization was performed at 56° C. for 24 h. Ultrathin sections were prepared, treated with a lead solution to provide contrast (Reynolds, 1963) and analysed using a JEOL 100B transmission electron microscope with an accelerating voltage of 80 kV (Jeol, Japan).

Results

Lycopene Induces Formation of Lipid Droplets and Boosts Mitochondria

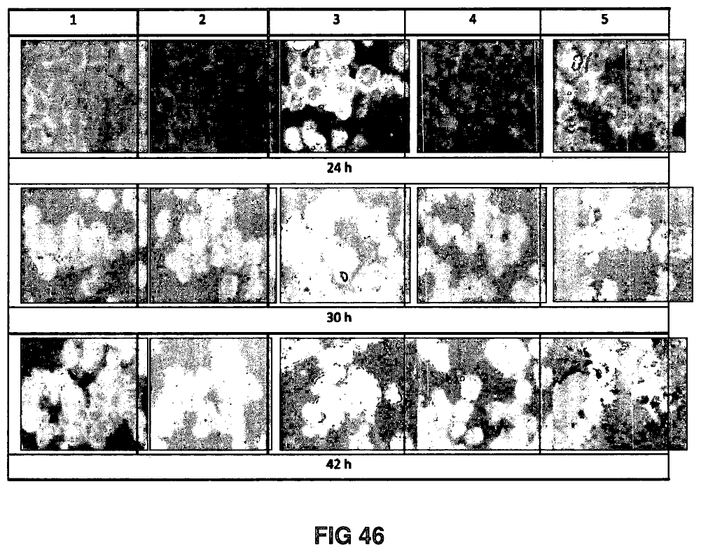

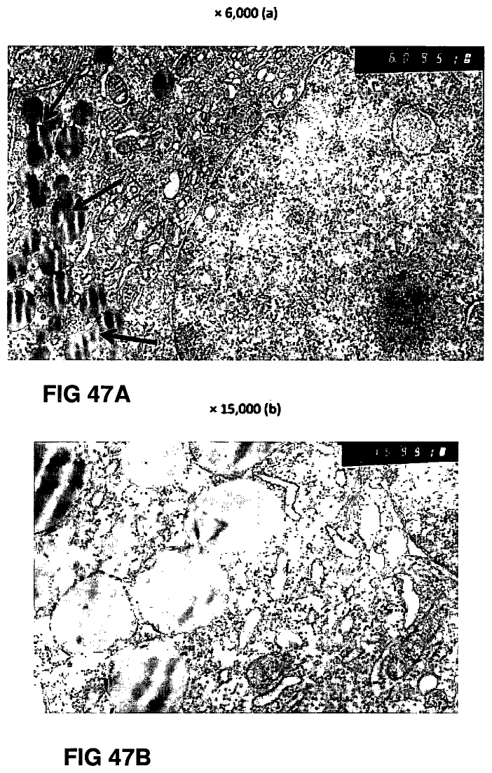

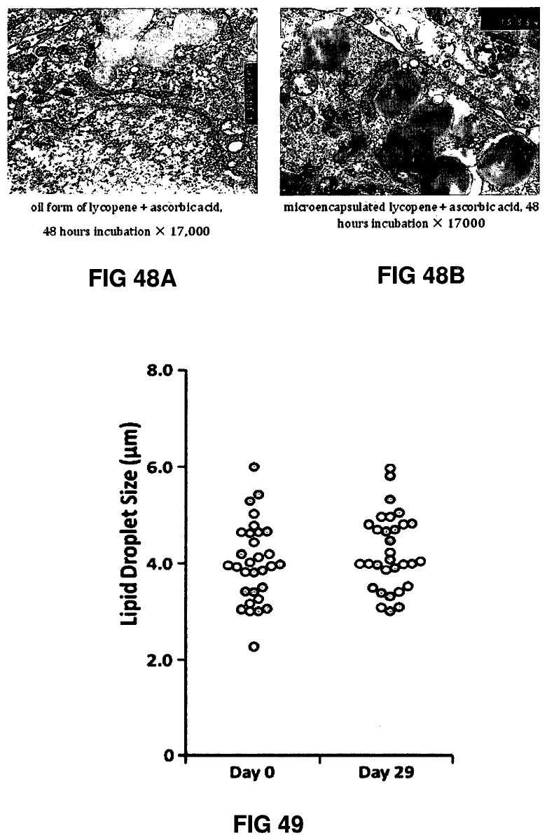

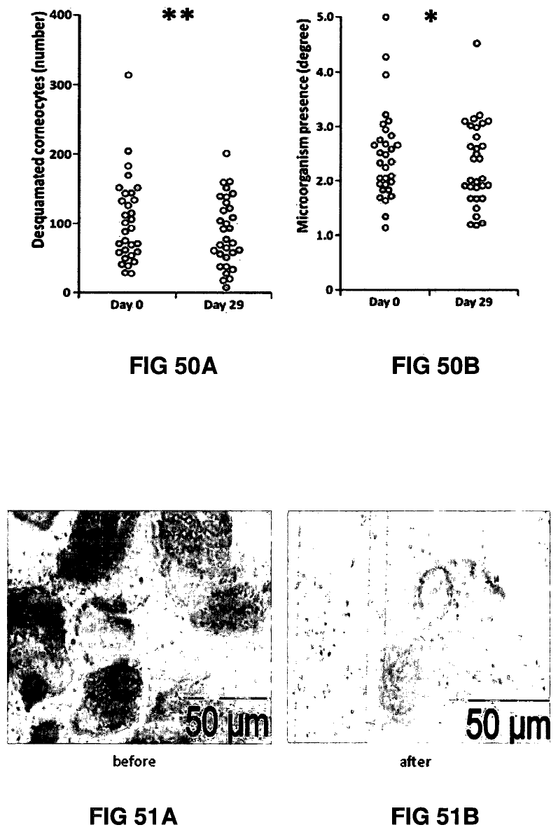

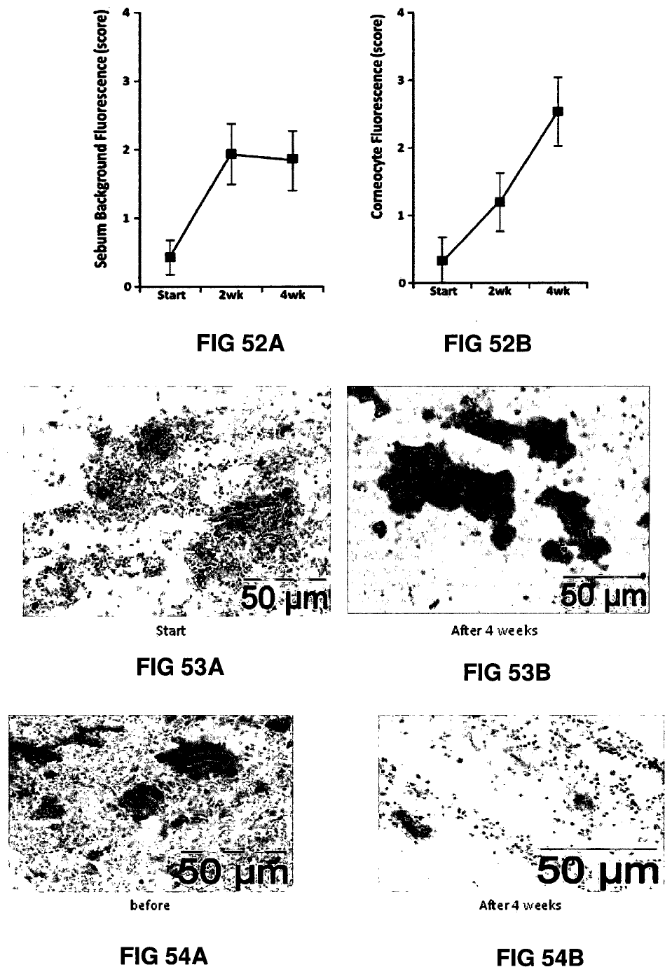

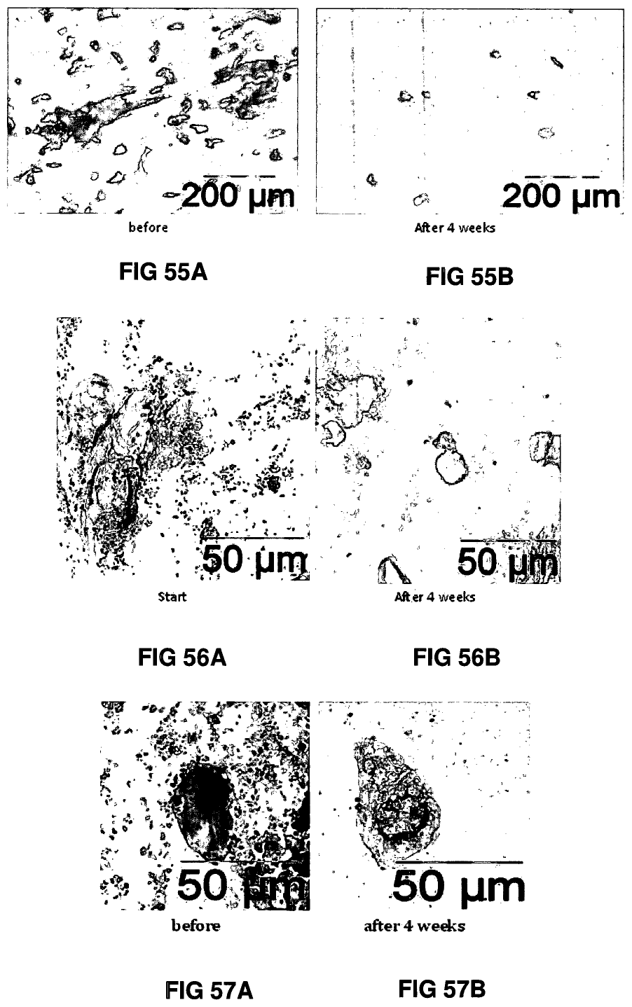

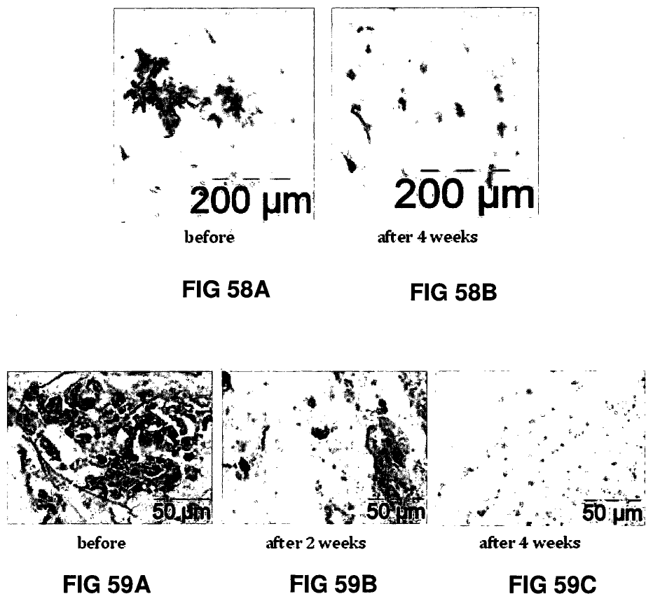

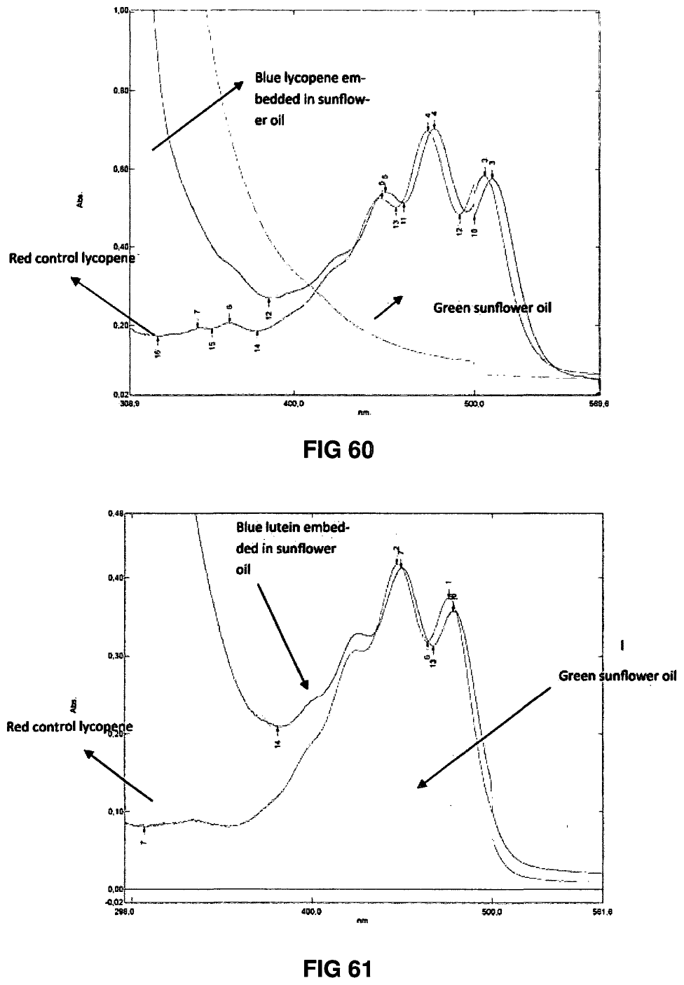

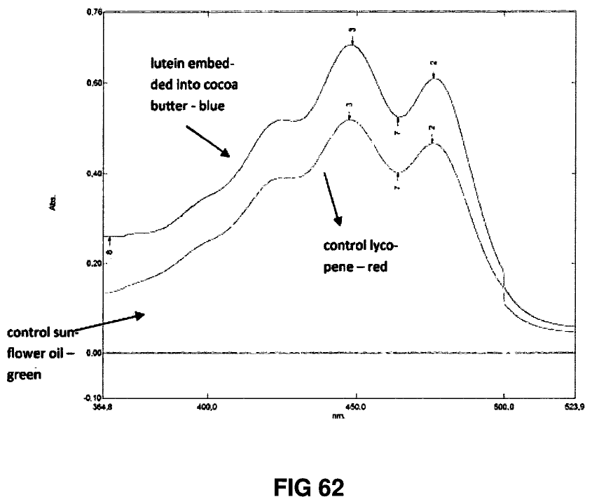

Formation of lipid droplets was evaluated in B10.MLM cells at 24, 30 and 42 hours following introduction into medium, oil-formulated or microencapsulated lycopene. Cell monolayers were stained with BODIPY fluorescent dye specific for lipids and staining results were evaluated using fluorescence microscopy. It was shown that inoculation with both forms of lycopene caused lipid droplet formation in B10.MLM cells only 24 hours after lycopene addition to the medium, FIG. 46. The number of cells, which were positive for lipid droplet formations, was progressively increased over the observation period. In the control cell monolayers, with starch and olive oil, there was no lipid droplet formation or inhibition of chlamydial growth, table 17.

TABLE 17
Stimulation of Lipid Droplets formation by incubation
cells with different lycopene formulations.

| | Total square of lipid droplets per 1 cell, | |
|---|---|---|
| | in micron² | |
| Alveolar macrophage cell line | 24 hs incubation | 42nd incubation |
| Control cells | 0 | 0 |
| Cells + oil formulated lycopene | 4.5 ± 0.9 | 21.9 ± 2.9 |
| | 2.9 ÷ 7.4 | 9.9 ÷ 27.8 |
| Cells + microencapsulated | 1.5 ± 0.8 | 8.3 ± 1.9 |
| lycopene | 0.7 ÷ 3.1 | 1.0 ÷ 12.1 |

Lipid droplet formation in the alveolar macrophage cell line was investigated by electron microscopy. The cells, following addition of oil form or microencapsulated lycopene had visible lipid particles, which were integrated in the membrane structure, FIG. 47A-B, 48A-B. Lipid particles were of moderate electron density with osmiophilic granules. There were a significant number of lipid droplets of moderate electronic density. These droplets contained multiple osmiophilic granules. The cisterns of the endoplasmic reticulum were extended and widened.

Mitochondrial structures were significantly increased both in size and their numbers, and they had a round-shaped or oval shape, FIG. 48A-B. This indicates not only on increased growth of mitochondria but also on the boost of their respiration activity.

### Example 24

In Vivo Studies

a) Carotenoids stimulate formation of larger particles of circulating lipoproteins, which results in increase of their capacity to transport molecular oxygen.

b) Carotenoids have anti-hypoxia activity and stimulate tissue oxygen saturation.

Material and Methods

Study Design

The study was conducted at the Institute of Cardiology, the Ministry of Health of the Russian Federation (Saratov, RF), during 2010-2011. The protocol was approved by the local ethics committee. All patients were informed about the purpose of the study and have given written consent regarding their participation in the study.

56 participants were recruited, 26 female and 30 male, of 45 to 72 years old.

Inclusion Criteria
(i) Caucasian male or female subjects 45-73 years old.
(ii) Signed informed consent.
(iii) Non- or light-to-moderate smokers 10 cigarettes daily).
(iv) Willingness and ability to comply with the protocol for the duration of the study.

Exclusion Criteria
(i) Unwillingness to sign informed consent.
(ii) Unable to comply with the protocol for the duration of the study.
(iii) History of MI in the 3 months preceding the study.
(iv) Ejection fraction (EF)<45%.
(v) Significant medical condition that would impact safety considerations (e.g., significantly elevated LFT, hepatitis, severe dermatitis, uncontrolled diabetes, cancer, severe GI disease, fibromyalgia, renal failure, recent CVA (cerebrovascular accident), pancreatitis, respiratory diseases, epilepsy, etc.).
(vi) Compulsive alcohol abuse (>10 drinks weekly), or regular exposure to other substances of abuse.
(vii) Participation in other nutritional or pharmaceutical studies.
(viii) Resting heart rate of >100 beats per minute or <45 beats per minute.
(ix) Positive test for tuberculosis, HIV, or hepatitis B.
(x) Did not tolerate phlebotomy.
(xi) On a special diet in the 4 weeks prior to the study (e.g., liquid, protein, raw food diet).
(xii) Tomato, green leaf vegetables or seafood intolerance.

Supplementation

All participants were randomised into 7 groups with 8 persons each.

Three lycopene products were chosen: Lyc-O-Red, LR (LycoRed, Switzerland), lacto-lycopene, L-lycopene (Nestle, Switzerland) and formulated lycopene (Lycotec., UK). The dose of each of them was 7 mg, and it was taken on a daily basis in 1 capsule with main meal.

Two lutein products were chosen: one from Holland & Barrett, UK, and formulated lutein from Lycotec, UK. The daily dose for the former was 12 mg, and for the latter 7 mg. The product was taken in a single capsule with main meal of the day.

Two astaxanthin products were chosen: one from Solgar, UK, and formulated astaxanthin from Lycotec, UK. The daily dose for the former was 10 mg, and for the latter 7 mg. The product was taken in a single capsule with main meal of the day.

The period of supplementation for all products lasted for 4 weeks.

Molecular Oxygen Capacity of Serum Lipoproteins

This parameter, as a function of their Hydrophobicity, was measured as described above.

Tissue Oxygenation

As a tissue target for the assessment of oxygen saturation, StO₂, or combined level of oxygenated haemoglobin and myoglobin, we used the Nar eminence and forearm muscles of the patients. StO₂ was analysed by continuous wavelength near-infrared spectroscopy, NIRS, with wide-gap second-derivative (In Spectra, Hutchinson Technology, MN, USA). The measurements were made at different time points. The recording was started after 15 min of rest in a supine position before occlusion of the brachial artery. It was then continued during stagnant ischemia induced by rapidly inflating the cuff to 50 mmHg above systolic BP. The ischemia lasted for 3 min, and the recording period lasted for another 5 min after that until StO₂ was stabilized [8, 9]. Then the area under the hyperaemic curve, AUC, of the recorded signal for the settling time in the post-occlusion period was calculated as described earlier in % O₂/minute [10, 11].

Blood Collection

Blood was collected in the morning after night fast from arm veins of the patients. The plasma was separated from the rest of the clotted mass by centrifugation, then aliquots were stored at −80-C prior to analysis.

Statistics

For the assessment of normally distributed parameters, the Shapiro-Wilkinson method was used. Student's t-test was then applied both for paired and unpaired samples.

In cases where parameters were not normally distributed Mann-Whitney U test and Kruskal-Wallis test were used.

ANOVA and ANCOVA were used with post hoc analysis (Statistica9 suit, StatSoft; Inc.). Statistical significance between two-tailed parameters was considered to be P<0.05.

Results

It was shown in that supplementation of people with different carotenoids, lycopene, lutein or astaxanthin could increase serum lipoprotein hydrophobicity and therefore the capacity for molecular oxygen. These changes in serum accompanied changes in peripheral tissue oxygenation, tables 18-20. Observed differences in changes of these parameters between groups who ingested even the same carotenoid, and even the same dose, could probably be explained by different formulations made by different suppliers of the products.

It was interesting to observe that the higher increase of oxygen carrying capacity of serum lipoproteins was the higher incremental tissue oxygenation was.

TABLE 18
Changes in the molecular oxygen capacity of serum lipoproteins and tissue oxygen
saturation caused by supplementation by different lycopene products for 4 weeks.

| | | O₂ Capacity of Serum Lipoproteins as | | | |
|---|---|---|---|---|---|
| | | a function of their Hydrophobicity, | | Tissue Oxygen Saturation StO₂, in | |
| | | in Eλ560hm × 10−3 | | AUC mm | |
| Products | n | 0 w | 4 w | 0 w | 4 w |
| L-Lycopene 7 mg | 8 | 842 ± 94 | 887 ± 91 | 73 ± 7.1 | 74 + 7.2 |
| | | | (Δ = +45) | | (Δ = +1) |
| | | | p > 0.05 | | p > 0.05 |
| LR Lycopene 7 mg | 8 | 652 ± 67 | 794 ± 83 | 69 ± 7.2 | 76.5 + 7.8 |
| | | | (Δ = +142) | | (Δ = +7.5) |
| | | | p < 0.05 | | p < 0.05 |
| GA Lycopene 7 mg | 8 | 1,011 ± 98 | 1,359 ± 95 | 63 ± 6.4 | 88 ± 8.9 |
| | | | (Δ = +348) | | (Δ = +25) |
| | | | p < 0.01 | | p < 0.01 |

TABLE 19
Changes in the molecular oxygen capacity of serum lipoproteins and tissue oxygen
saturation caused by supplementation by different lutein products for 4 weeks.

| | | O₂ Capacity of Serum Lipoproteins as | | | |
|---|---|---|---|---|---|
| | | a function of their Hydrophobicity, | | Tissue Oxygen Saturation StO₂, in | |
| | | in Eλ560hm × 10−3 | | AUC mm | |
| Products | n | 0 w | 4 w | 0 w | 4 w |
| Lutein 12 mg | 8 | 675 ± 73 | 804 ± 76 | 66 ± 7.4 | 69 + 7.1 |
| | | | (Δ = +129) | | (Δ = +3) |
| | | | p > 0.05 | | p > 0.05 |
| GA Lutein 10 mg | 8 | 710 ± 73 | 1,107 ± 103 | 50 ± 4.9 | 66 ± 6.2 |
| | | | (Δ = +397) | | (Δ = +16) |
| | | | p < 0.01 | | p < 0.01 |

TABLE 20
Changes in the molecular oxygen capacity of serum lipoproteins and tissue oxygen saturation
caused by supplementation by different astaxanthin products for 4 weeks.

| | | O₂ Capacity of Serum Lipoproteins as | | | |
|---|---|---|---|---|---|
| | | a function of their Hydrophobicity, | | Tissue Oxygen Saturation StO₂, in | |
| | | in Eλ560hm × 10−3 | | AUC mm | |
| Products | n | 0 w | 4 w | 0 w | 4 w |
| Astaxanthin 10 mg | 8 | 622 ± 59 | 714 ± 71 | 63 ± 6.2 | 65 + 6.1 |
| | | | (Δ = +92) | | (Δ = +2) |
| | | | p > 0.05 | | p > 0.05 |
| GA Astaxanthin 7 mg | 8 | 603 ± 65 | 789 ± 93 | 68 ± 5.7 | 75 ± 6.9 |
| | | | (Δ = +186) | | (Δ = +7) |
| | | | p < 0.01 | | p < 0.01 |

### Example 24a

In Vivo Studies

Carotenoids increase size of sebum droplets on the skin, reduce its viscosity and stimulate declined sebum production and functions: from skin lubrication and prevention of its dehydration to skin defense and ability to control its microbiota.

Methods and Materials

Study Design This collaborative study was undertaken by Lycotec Ltd (Cambridge, UK) and the Institute of Cardiology of the Ministry of Health of the Russian Federation (Saratov, Russian Federation). The enrolment of study participants was carried out in Saratov, from the existing pool of healthy volunteers. The study protocol was approved by the local Ethics Committee (FGBU SarNIIK 18.02.2014) guaranteeing that the study conformed to the European Medicines Agency Guidelines for Good Clinical Practice. All volunteers who agreed to participate in the study were informed about the purpose of the study and provided written informed consent. All samples collected during the study were analysed in the laboratory of Lycotec Ltd in Cambridge, UK.

Inclusion and Exclusion Criteria

The following inclusion criteria were applied to all individuals who volunteered to take part in the study:

i) Only clinically healthy adult Caucasian males and females over the age of 40 were considered for recruitment;

ii) Signed informed consent forms;

iii) Non-smokers or light/moderate smokers 10 cigarettes per day);

iv) Not under drug treatment;

v) Willing and able to comply with the protocol for the duration of the study.

The following exclusion criteria were applied:

i) Unwillingness to sign informed consent forms;

ii) Inability to comply with the protocol for the duration of the study;

iii) The presence of a significant medical condition (diagnosed cardiovascular or cerebrovascular disease, diabetes mellitus, oncological conditions etc) or a disorder affecting skin (such as psoriasis, pronounced acne and allergic skin conditions);

iv) On-going drug treatment (especially hormonal therapy);

v) Excessive alcohol consumption (over 35 UK Units per week);

vi) Simultaneous participation in other studies involving dietary or pharmaceutical interventions.

Study Subjects and Dietary Intervention

The total number of volunteers recruited for the study was 54.

Group I—31 (17 male and 14 female subjects, 40-80 years of age) for astaxanthin intervention;

Group II—15 middle-aged (7 males and 8 females, 48-65 years old) for lycopene intervention and

Group III—8 middle-aged (4 males and 4 females, 50-67 years old) for lutein intervention.

All study participants initially had their body mass index (BMI) determined by measuring their body mass and height in the morning and then calculating the index in kg/m². Individuals with BMI below 25 kg/m² were classified as subjects with normal weight, those with BMI in the range between 25 kg/m² and 30 kg/m² were classified as overweight, and BMI values over 30 kg/m² indicated obesity.

Group I—participants received 4 mg daily doses of formulated astaxanthin,

Group II—participants received 7 mg formulated lycopene,

Group III—participants received a combination of 7 mg GA Lutein and 1.4 mg Zeaxanthin.

Lycotec Ltd, Cambridge, UK, manufactured all products.

Daily dose of each carotenoid product was taken in one capsule with the main evening meal for 4 weeks (from day 1 to day 28 of the study).

Sample Collection and Preparation

For RSSC sample collection all participants of the study were requested to avoid facial hygienic manipulations for 24 hours before material collection, which was carried out in the morning. These samples were collected only before initiation of astaxanthin supplementation (day 0) and at the end of the study (day 29). RSSC sample collection and preparation was performed according to previously described procedures [25,28]. Briefly, RSSC samples were collected using polyester swabs from the surface of the facial skin (the sides of the nose). During the procedure two samples were taken (one swab per side). Each collected sample was placed on the surface of a microscope slide. A second microscope slide was pressed against the surface of the first one. This procedure provided a pair of identical smears, which did not require fixation. All slides with collected samples (i.e. four slides per study participant per time point) were coded to provide sample anonymity for blinded analysis. All collected samples were sent to the laboratory of Lycotec Ltd for further processing and eventual microscopic examination.

Sample Analysis

For morphological analysis of RSSC samples one slide of the first pair was stained with hematoxylin and eosin in order to identify any cells or cell remnants. The second slide was stained using Oil Red O (Lipid Stain, ab150678, Abcam, Cambridge, UK) for lipid visualization and lipid droplet size evaluation. One slide from the other pair was stained with crystal violet solution (Gram staining) to assess the level of microorganism presence. The remaining slides were kept unstained for possible future use.

All stained smears were examined microscopically by a highly experienced cytopathologist. Microscopy was performed using Olympus BX41 laboratory microscope (Olympus, Japan) at ×1000 magnification.

The analysis included examining 40 fields of view within one smear. Dark field microscopy was applied for lipid crystal visualization. Photomicrographs were prepared using Olympus DP71 camera.

The analysis of typical structural elements of the RSSC comprised lipid droplet measurements, counting characteristic lipid crystals and desquamated corneocytes, and evaluation of bacterial presence as previously described [12, 13]. The indicated micro objects/structures were quantified using Cell\hat{ }B imaging software (Digital Imaging Solutions, Olympus, Japan). All microscopic samples were blinded before their examinations.

Lycopene Detection in Facial Sebum and Desquamated Corneocytes by Immunofluorescent Staining

Smears collected from the surface of the facial skin were fixed with methanol. Permeabilised corneocytes and sebum present in the material were stained for direct immunofluorescence using fluorescein isothiocyanate-conjugated monoclonal antibody against lycopene recently generated by our group [14]. Fluorescent staining was visualised using Nikon Eclipse 50i microscope with fluorescence attachment. The semi-quantitative analysis was based on visually assessing fluorescence levels in corneocytes and surrounding sebum in 20 random fields of view at ×200 magnification. Fluorescence intensity in the samples was classified using the following scoring system: 0—no fluorescence; 0.5—traces of fluorescence; 1—weak fluorescence; 2—moderate fluorescence; 3—strong fluorescence of some cells or areas of sebum background; 4—extremely strong fluorescence (confluent fluorescent elements within corneocytes). Fluorescence assessment in each sample was repeated blindly three times.

Data Analysis

All quantitative results were calculated for the whole study population as well as for male and female subjects separately. Separate result assessments were also carried out for subgroups of subjects defined according their BMI (normal weight, overweight, and obese).

In the morphological analysis of the RSSC quantitatively assessed parameters comprised lipid droplet size, numbers of desquamated corneocytes and lipid crystals and bacterial presence estimates (using Bacterial Presence Assessment Scale as described in our previous paper [12]).

Results of all quantitative measurements (or counting) were compared between the time points of the study. For MDA analysis comparisons were made between days 0, 15, and 29. For RSSC assessment only initial and final time points (i.e. days 0 and 29) were compared. Descriptive statistics were used. Mean, standard deviation, median and range values as well as 95% confidence intervals were determined. Paired t-test (two-sided P-values calculated) was applied to determine statistical significance for the differences between time points. T-test for independent means was used for comparisons between subgroups. Scatter diagrams were employed for presenting individual results for all measurements in the RSSC assessment. Bar charts were used for presenting comparisons of group means. All data handling and statistical analyses were carried out using IBM SPSS 19.0 statistical package (IBM Inc., Armonk, N.Y., USA).

Results

Astaxanthin Trial

Lipid Droplet Size

FIG. 49 demonstrates the results of lipid droplet size measurements before astaxanthin intake initiation (day 0) and at the end of the study (day 29). Despite some variability of the results, it appeared that there was a relatively weak trend to increasing lipid droplet size at the end of the study. The difference between the two time points tested using paired t-test, however, has not reached statistical significance (P=0.0683). The results also showed that there was no gender-dependent difference in lipid droplet size. Separate analysis of subgroups with different body composition revealed no statistically significant effect of astaxanthin in volunteers with normal weight or overweight individuals, whereas in the subgroup of obese subjects a statistically significant (P=0.0214) increase of lipid droplet size was detected.

Corneocyte Desquamation

Evaluation of corneocyte desquamation in samples taken before astaxanthin consumption (day 0) and after four weeks of astaxanthin intake (day 29) has revealed a clear trend that is evident from FIG. 50A. Corneocyte desquamation levels at the end of the study were clearly reduced in most volunteers. This phenomenon was convincingly confirmed by paired t-test, which was highly significant (P=0.0075). In some volunteers the difference in desquamation levels was easy to see during microscopic analysis (FIG. 51A-B). When BMI-defined subgroups were assessed separately, the outcome of paired t-test was statistically significant only for obese individuals (P=0.0473). No gender-related difference was revealed.

Microbial Presence—Gram-Positive Microbiota

The analysis of the microbial presence in RSSC samples was conducted using our previously described [12] semi-quantitative scale. The obtained results are presented in FIG. 50B. We were able to find a statistically significant (P=0.0367) decrease of this parameter at the end of the study for the whole volunteer group. In addition, a statistically significant (P=0.0312) decrease in microbial presence was detected among obese volunteers. No gender-related differences could be found.

Lycopene Trial

Lycopene Measurement in the Serum and Material Collected from the Surface of the Skin During Dietary Supplementation with Lycopene

Results of the study that included 15 ‘middle-aged’ volunteers receiving dietary supplementation with lycopene for 4 weeks are presented in FIGS. 52A-B and 53A-B.

Gradual accumulation of lycopene during supplementation was evident in the material collected from the surface of the facial skin. However, there was a difference between desquamated corneocytes and unstructured sebum. Lycopene presence in desquamated corneocytes significantly increased for the whole period of supplementation (FIG. 52B). In contrast, sebum assessment could reveal lycopene amount growth only during the first two weeks of supplementation. No further increase in lycopene presence in the sebum was observed by the end of the study (FIG. 52A).

The observed increase of lycopene in sebum was accompanied by a significant increase of the sebum production, which is typically depressed in ageing or stressed skin [12, 13]. Moreover, not just the quantity of the sebum improved but its quality too; the diameter of the sebum droplets was enlarged and its viscosity decreased, FIG. 53A-B. It seems that these positive changes in sebum resulted in improvement in its skin defensive properties, which was manifested in the observed significant reduction of the bacterial load on the surface of the skin, FIG. 54A-B.

Combination of these positive developments, caused by lycopene incorporation into the sebum, resulted in improving health of corneocytes. Their rate of desquamation was noticeably lower, and clusters of these cells, which present cross-linked damaged corneocytes, observed in FIG. 55A, were not detectable in the analysed material in the end of the trial FIG. 55B.

Lutein Trial

Supplementation of middle-aged people with a combination of lutein and zeaxanthin for 4 weeks resulted in a similar improvement of quantity and quality of their sebum, FIG. 56A-B.

### Example 24b

In a Separate Study, the Following was Used:

Materials and Methods

Study Design. The total number of volunteers recruited to take part in the study was 18 (9 male and 9 female subjects) Caucasians 40-67 years old.

Inclusion Criteria were:
ability to sign an informed consent,
light-to-moderate smokers 0 cigarettes daily),
moderately obese with BMI between 30 and 35 kg/m2,
with elevated serum markers of inflammatory oxidative damage, IOD ≥40 μM/mL and oxidative stress, LDL-Px, ELISA×10³≥200,
no participation in other dietary trials during the last 3 months before enrolment and duration of study,
willingness and ability to comply with the study protocol for the duration of the study.

Exclusion criteria were:
unwillingness to sign informed consent,
unable to comply with the protocol for the duration of the study,
history of myocardial infarction in the 3 months preceding the study, ejection fraction (EF)<45%,
significant medical condition that would impact safety considerations (e.g., significantly elevated LFT, hepatitis, severe dermatitis, uncontrolled diabetes, cancer, severe GI disease, fibromyalgia, renal failure, recent CVA (cerebrovascular accident), pancreatitis, respiratory diseases, epilepsy, etc.),
compulsive alcohol abuse (>10 drinks weekly),
or regular exposure to other substances of abuse,
participation in other nutritional or pharmaceutical studies,
resting heart rate of >100 beats per minute or <50 beats per minute,
positive test for tuberculosis, HIV, or hepatitis B,
unable to tolerate phlebotomy,
special diets in the 4 weeks prior to the study (e.g., liquid, protein, raw food diet),
tomato intolerance.

Products Formulated with Lycopene was developed and made by Lycotec Ltd. (Cambridge, United Kingdom). The daily dose was 1 capsule of 20 mg of lycopene, and it was taken with the main meal of the day. The period of administration was 1 month.

Methods.

BMI, Pulse Rate, and BP.

Measurements of body mass index, BMI, body mass of the patients and their height were carried out in the morning and BMI was calculated in kg/m2. Pulse rate, systolic and diastolic blood pressure, SBP and DBP, were recorded three times on the left arm of the seated patient after 15 min of rest. The time between measurements was greater than 2 minutes. The mean result for each parameter was calculated. All body and vascular parameters were recorded in the morning between 8 and 10 am.

Tissue Oxygenation.

Thenar eminence and forearm muscles of the patients were used as a tissue target for the assessment of oxygen saturation, StO₂, or combined level of oxygenated haemoglobin and myoglobin. StO₂ was assessed by continuous wavelength near-infrared spectroscopy, NIRS, with wide-gap second-derivative (In Spectra, Hutchinson Technology, MN, USA). The measurements were taken at different time points.

The recording was initiated after 15 min of rest in a supine position before occlusion of the brachial artery. It was then continued during stagnant ischemia induced by rapidly inflating the cuff to 50 mm Hg above systolic BP. The ischemia lasted for 3 min, and the recording period lasted for another 5 min after that until StO2 was stabilized. The area under the hyperaemic curve, AUC, of the recorded signal for the settling time in the post-occlusion period was then calculated as described earlier in % O₂/minute [34, 35].

Samples Collection.

Blood was collected by phlebotomy in the morning, in the hospital, from the arm veins of patients following night fast. The serum was separated from the rest of the clotted mass by centrifugation; aliquots were then stored in code-labelled tubes for blinded analysis and stored at −80° C. until use.

For sample collection from the surface of the facial skin and samples of the cerumen all study participants were requested to avoid facial and ear hygienic manipulations for 24 hours before sampling, which was carried out in the morning in parallel with blood sample collection. Skin surface sample collection and preparation was performed as previously described. Briefly, samples were collected using polyester swabs from the surface of the facial skin (the sides of the nose). During the procedure two samples were taken (one swab per side). Each collected sample was placed on the surface of a microscope slide. A second microscope slide was pressed against the surface of the first one. This procedure provided a pair of identical smears. All slides with collected samples were coded to provide sample anonymity for blinded analysis and stored at −20° C. until further analysis.

Lycopene Quantitative Analysis.

The lycopene concentration in all serum samples was measured in duplicate by high-performance liquid chromatography with modifications. Briefly, 400 μl of serum was mixed with 400 μl of ethanol and was extracted twice with 2 ml hexane. The combined hexane layers were evaporated to dryness in a vacuum (Scan Speed 32 centrifuge) and the residue reconstituted to a volume of 100 μl in sample solution (absolute ethanol—methylene chloride, 5:1, v/v). The specimens were centrifuged again (15 minutes at 10,000 g) and clear supernatant was transferred to HPLC vials. Five microliters of the extract was injected into an Acquity HSS T3 75×2.1 mm 1.8 μm column (Waters, USA) preceded by a Acquity HSS T3 1.8 μm VanGuard precolumn (Waters, USA) and eluted isocratically at 45° C. with the mobile phase (acetonitrile—0.08% phosphoric acid solution—tert-Butyl methyl ether, 70:5:25, v/v/v) at a flow rate of 0.5 ml/min. The lycopene peak was detected by a Photodiode Array Detector (Waters, USA) at 474 nm. The peak area was measured using Empower 3 software (Waters, Mass.). The lycopene concentration in serum samples was calculated by reference to an analytical standard (lycopene from tomato, L9879, Sigma, USA).

Inflammatory Oxidative Damage (IOD).

Serum samples were incubated overnight in 0.05 M PBS acetate buffer (pH 5.6) to imitate the type of oxidative damage, which occurs during the release of lysosomes following neutrophil degranulation. The following morning the reaction was stopped using trichloroacetic acid. The concentration of the end products such as malonic dialdehyde (MDA), and other possible thiobarbituric acid reactive substances (TBARS), was then measured by colorimetry using reagents and kits from Cayman Chemical (MC, USA).

LDL-Px and Lipoprotein O2. Activity of serum LDL peroxidase proteins, which include IgG with superoxide dismutase activity, was measured as described previously. Plasma oxygen, which carried by blood lipids/lipoproteins was measured by catalymetry.

Statistics.

For the assessment of normally distributed parameters the Shapiro-Wilk method was used. Student's t-test was then applied for both paired and unpaired samples. In cases where parameters were not normally distributed the Mann-Whitney test and Kruskal-Wallis test were used. ANOVA and ANCOVA were used with post hoc analysis (Statistica 9 suite, StatSoft; Inc.). Statistical significance between two-tailed parameters was considered to be P<0.05.

Results

General Characteristics of the Study Population:

Baseline characteristics of the participants are presented in table 21. Apart of their increased BMI all other measured parameters, from cardiovascular to blood biochemistry, were within the norm.

TABLE 21
BASELINE CHARACTERISTICS OF THE ENROLLED VOLUNTEERS
(Mean +/− SD)

| | |
|---|---|
| Number of Patients | 18 |
| Males | 9 |
| Females | 9 |
| Age | 54.8 ± 5.6 |
| Light/Moderate Smokers | 3 |
| Body Mass Index in kg/m² | 32.5 ± 3.1 |
| Fasting Glucose mmol/dL | 5.6 ± 0.42 |
| Total Cholesterol mg/dL | 183 ± 15.1 |
| Triglycerides mg/dl | 136 ± 13.8 |
| LDL, in mg/dL | 134 ± 11.3 |
| HDL, in mg/dL | 49.2 ± 2.9 |
| Pulse rate per min | 67.8 ± 3.6 |
| Blood Pressure, in mm Hg | |
| Systolic | 121.3 ± 7.8 |
| Diastolic | 77.7 ± 4.9 |

Blood and Tissue Parameters

Supplementation with 20 mg formulated lycopene for one month resulted in more than 2 fold increase in its serum concentration and 3 fold in the cerumen (table 22).

TABLE 22
Changes in blood and tissue parameters after supplementation with

| | Daily dose of lycopene 20 mg (n = 18) | |
|---|---|---|
| Parameters | before | after |
| Lycopene in serum, in ng/ml | 137 ± 14.5 | 310 ± 29.9 |
| | | p < 0.001 |
| Lycopene in cerumen, in ng/mg | 0.29 ± 0.05 | 0.90 ± 0.12 |
| | | p < 0.001 |
| Triglycerides mg/dL | 136 ± 10.1 | 128 ± 9.7 |
| | | p < 0.05 |
| LDL, in mg/dL | 134 ± 11.3 | 121 ± 10.5 |
| | | p < 0.05 |
| HDL, in mg/dL | 49.2 ± 2.9 | 49.9 ± 3.0 |
| | | p > 0.05 |
| IOD, in μM MDA | 107 ± 11.6 | 60 ± 5.8 |
| | | p < 0.001 |
| LDL-Px, in ELISA × 10³ | 545 ± 49 | 286 ± 29 |
| | | p < 0.001 |
| Lipoprotein O₂, in μM | 2.94 ± 0.25 | 3.44 ± 0.27 |
| | | p < 0.001 |
| StO₂, in AUC mm | 64.1 ± 4.9 | 80.4 ± 5.3 |
| | | p < 0.05 |

This increase was accompanied by significant changes in the lipid profile. Concentration of LDL and triglycerides was reduced by 13 mg/dL and 8 mg/dL accordingly. However, level of HDL was not significantly affected. Concentrations of glucose and liver enzymes, ALT and AST, were also not changed by the end of the trial (results are not presented).

However, supplementation by formulated lycopene resulted in a significant inhibition of markers of oxidative damage and inflammation. By the end of the month, both parameters were reduced by about 50%.

There were also improvements in the molecular oxygen metabolism. By the end of the trial its transportation by blood lipoproteins were increased by 17% and tissue oxygenation in skeletal muscle was boosted by 25% (table 22).

Skin Parameters

Supplementation of the participants with 20 mg of formulated lycopene for one month resulted in significant rejuvenation of sebum and corneocytes. The viscosity of the former, in terms of the size of the lipid droplets collected from the surface of the skin, was increased during this trial. The rate of corneocyte exfoliation was reduced by about 17%. Moreover, the level of their damage, in terms of the number of the cross-linked clusters of these cells, was reduced by 70% (table 23).

It was interesting to observe that these improvements of the sebum and corneocyte parameters were accompanied by a significant reduction of the total load of the gram-negative bacteria on the surface of the skin (table 22).

TABLE 23
Changes in bacteria, sebum and corneocyte parameters
of the skin after supplementation with 20 mg
formulated lycopene for one month.

| | Daily dose of lycopene 20 mg (n = 18) | |
|---|---|---|
| Parameters | before | after |
| Sebum droplet size, in μm | 3.87 ± 0.27 | 4.09 ± 0.40 |
| | | p < 0.05 |
| Corneocyte exfoliation rate* | 84 ± 8.9 | 72 ± 11.1 |
| | | p < 0.01 |
| Corneocyte damage** | 4.70 ± 1.53 | 2.79 ± 0.84 |
| | | p < 0.01 |
| Bacteria load*** | 2.34 ± 0.29 | 2.11 ± 0.21 |
| | | p < 0.05 |

*as an average number of single corneocytes in stratum cornea,
**as an average number of cross-linked damaged corneocyte clusters in stratum cornea;
***score of gram negative bacteria staining, each parameter was calculated in 40 randomly selected microscopic areas (×1,000)

The fact that lycopene can be secreted to the surface of the human body either with the cerumen, which we are reporting here, or sebum (results are not presented), have, to the best of our knowledge, not been published.

Sebum is essential not only for skin lubrication, which prevents it from dehydration, but also is an important part of its immune system and its anti-bacterial Acid Mantle, but also supplying antioxidants and perhaps other beneficial molecules to the surface of the skin. It has been reported that with ageing the quality of the sebum, and in particular its viscosity, is increased, which is accompanied by accelerated corneocyte desquamation and an increase of the bacterial load on the surface of the skin. In our study we observed that supplementation of the skin with lycopene of the middle-aged persons resulted in the restoration of the sebum viscosity, reduction of the corneocyte damage and desquamation, and also by reducing the skin bacteria overgrowth.

### Example 25

Clinical Case

Carotenoids increase quantity size of sebum droplets on the skin, improve its quantity and quality which translates into better protection of corneocytes and more effective treatment of skin inflammation and the damage caused by bacteria infection.

Clinical Case

A micro-abscess was identified on the face of a man who was 62 years old, with a body mass 80 kg and height 178. A supplementation with formulated lycopene 7 mg in 1 capsule per day with main meal for 4 weeks was proposed.

Serum and skin parameters we analysed as described above. The results were obtained before, on the 2^{nd} and at the end of supplementation.

Results

Blood Changes

After two weeks of supplementation lycopene concentration in serum was increased from 340 nm/ml to 470 nm/ml. By the end of the trial it reached 580 nm/ml.

This was accompanied by steady reduction of the level of inflammatory oxidative damage from 311 nm of MDA/ml, which detected in the beginning of the treatment, to 274 nm MDA/ml at the week 2 and to 199 nm MDA/ml by the end of the trial.

Skin Changes

These blood changes were accompanied by increase in the sebum production and improvement of its quality—enlargement of its droplets, which indicates the reduction of its peroxidation and as a result of this its viscosity—appearance of red droplets FIG. 57A-B (darker spots on a light grey background).

The positive changes in blood and improvement of the sebum during lycopene supplementation translated into reduction of the rate of the exfoliation of corneocytes and disappearance of clusters of their damaged forms, which can be FIG. 58A compared to FIG. 58B (no damaged forms).

The monitoring of the level of the gram-positive bacteria and neutrophils, inflammatory response of the body on the skin infection, demonstrated the positive therapeutic dynamics of lycopene treatment, which resulted in complete recovery of the skin by the 4^{th} week, FIG. 59A-C.

In this clinical case lycopene supplementation resulted not only in the improvement of the quality and the quantity of the sebum and better protection of the corneocytes but also in effective treatment of the skin bacteria infection and its accompanying inflammatory damage.

### Conclusion

These results indicate that formation of complexes of lipids with incorporated of carotenoids can stimulate formation of lipid droplets, which can not only provide mitochondria with increased amounts of molecular oxygen but with energy-rich fatty acids. This may boost mitochondria growth and respiration which is important to stimulate:
non-shivering thermogenesis, and help to activate formation of beige fat cells;
ATP/energy production, and help to stimulate a range of cellular and tissues functions from regeneration and immunity to physical and cognitive performance;
shift from glycolysis to aerobic metabolism.

In addition, increase of hydrophobicity and consequently molecular oxygen capacity of serum/plasma lipoproteins can stimulate delivery of O₂ to the peripheral tissues to boost their respiration and have anti-hypoxia effect.

Formation of sebum fat complexes with carotenoids may result in an increase in its droplet size and overall sebum production, which is reduced in ageing and in skin stress. Restoration of the quantity and quality of the sebum may lead to improvement in its lubricating, prevention from dehydration and skin protective properties. As a consequence of this:
the health of corneocytes may improve and the rate of their desquamation may reduce,
the antibacterial defence of the skin may increase and its bacterial load may decrease,
more effective control of inflammatory responses to the skin damage.

Moreover, reduction of the viscosity of the sebum could help to prevent and treat acne and other conditions when high viscosity sebum cannot pass through skin pores, which may result in their clogging.

Reduction of the viscosity of adipose tissues, by increasing carotenoid incorporation into their lipid storage, may help to encourage patients to follow their liposuction treatment.

Moreover, this reduction of the viscosity of these tissues may help to improve their microcirculation, supply them with nutrients and oxygen, and hence improve their metabolism and respiration. This may reduce sub-clinical inflammation and sub-clinical hypoxia in these tissues as a part of prevention and treatment of obesity and cellulite.

### REFERENCES

1. G. Bacic, T. Walczak, F. Demsar, H. M. Swartz—Magnetic Resonance in Med. (1988), 8, 20-219.
2. D. A. Windrem and W. Z. Plachy—Biochem. Biophys. Acta (1980), 600, 655-665.
3. L. C. Clark Jr—Trans. Amer. Soc. Ant. Int. Organs 2 (1956), 4-57.
4. C. Pourplauche, V. Larreta-Garde and D. Thomas—Anal. Biochem. (1991), 198, 160-164.
5. R. R. Wise and A. W. Nayler—Anal. Biochem. (1985), 146, 260-269.
6. I. M. Petyaev and J. V. Hunt—Biochim. Biophys. Acta (1997), 1345, 293-305.
7. I. M. Petyaev, Vuylsteke A., Bethune D. W, Hunt J. V. —Plasma oxygen during cardiopulmonary bypass: a comparison of blood oxygen levels with oxygen present in plasma lipid. —Clinical Science (1997), v.94, 35-41.
8. A. Siafaka, E. Angelopoulos, K. Kritikos et al., “Acute effects of smoking on skeletal muscle microcirculation monitored by near-infrared spectroscopy,” Chest, vol. 131, no. 5, pp. 1479-1485, 2007.
9. P. L. Madsen and N. H. Secher, “Near-infrared oximetry of the brain,” Progress in Neurobiology, vol. 58, no. 6, pp. 541-560, 1999.
10. R. Bezemer, A. Lima, D. Myers et al., “Assessment of tissue oxygen saturation during a vascular occlusion test using nearinfrared spectroscopy: the role of probe spacing and measurement site studied in healthy volunteers,” Critical Care, vol. 13, supplement 5, pp. 1-7, 2009.
11. H. Gomez, J. Mesquida, P. Simon et al., “Characterization of tissue oxygen saturation and the vascular occlusion test: influence of measurement sites, probe sizes and deflation thresholds,” Critical Care, vol. 13, supplement 3, pp. 1-7, 2009.
12. N. E. Chalyk, T. Y. Bandaletova, N. H. Kyle and I. M. Petyaev “Age-related differences in morphological characteristics of residual skin surface components collected from the surface of facial skin of healthy male volunteers”—Skin Research and Technology 2017; 23: 212-220.
13. N. E. Chalyk, T. Y. Bandaletova, N. H. Kyle and I. M. Petyaev “Morphological characteristics of residual skin surface components collected from the surface of facial skin in women of different age”—Annals of Dermatology 2017 (4)—in press.
14. Valeriy V. Tsibezov, Yuriy K. Bashmakov, Dmitry V. Pristenskiy, Naylia A. Zigangirova, Ludmila V. Kostina, Natalya E. Chalyk, Alexey Y. Kozlov, Elena Y. Morgunova, Marina P. Chernyshova, Marina V. Lozbiakova, Nigel H. Kyle, and Ivan M. Petyaev Generation and Application of Monoclonal Antibody Against Lycopene. —MONOCLONAL ANTIBODIES IN IMMUNODIAGNOSIS AND IMMUNOTHERAPY, 2017, 36 (2), 62-67.

1. A particle or complex comprising hydrocarbon molecules and carotenoids wherein said carotenoids are embedded in the hydrocarbon molecules.

2. (canceled)

3. The particle or complex according to claim 1 wherein said hydrocarbon is a lipid.

4. The particle or complex according to claim 3, wherein the lipid is selected from a fatty acid, monoglyceride, diglyceride, triglyceride or other glycerolipid, phosphatic acid, phosphatidylethanolamine, phosphatidylcholine, phosphatidylserine, phosphatidylinositol or other glycerophospholipids, ceramide, sphingolipid, sterol, wax, fat-soluble vitamin, prenol, saccharolipid, polyketide and derivative thereof.

5. The particle or complex according to claim 1, wherein the carotenoid is selected from a lycopene, lutein, zeaxanthin, meso-zeaxanthin, astaxanthin, b-carotene and other carotenes, cryptoxanthins, flavoxanthin, neoxanthin and a tetraterpenoid.

6. The particle or complex according to claim 3, wherein the ratio carotenoid:lipid is from 1:100 to 1:1,000,000.

7. The particle or complex according to claim 3, wherein the lipid is in the form of droplets, globules or assembled proteins, and wherein the size of said droplets in the particle is increased at least by 50% compared to the size of a control particle that does not include a carotenoid.

8. The particle or complex according to claim 7, wherein the size of said droplets, globules or assembled proteins on the surface of an aqueous solution is increased at least by 20% compared to the size of a control particle that does not include a carotenoid.

9. (canceled)

10. The particle or complex according to claim 3, having one or more of the following properties altered compared to a control hydrocarbon that does not include a carotenoid: molecular packing density, thermodynamic properties, increase in enthalpy by at least 1%, increase in entropy by at least 1%, red-shift by at least 1 nm and hyperchromism by at least 2%.

11. (canceled)

12. The particle or complex of claim 3, being in a form of an emulsion, a cream, or colloid dispersion.

13. A food product comprising the particle or complex according to claim 2.

14. The food product according to claim 13, selected from a diary product.

15-16. (canceled)

17. The food product according to claim 13, comprising at least 1% fat (w/w).

18. A method for forming particles or complex, each comprising hydrocarbon and carotenoid molecules, wherein said carotenoid molecules are embedded in the hydrocarbon molecules, the method comprising the step of providing hydrocarbon and carotenoid molecules, disrupting folding of the hydrocarbon molecules by embedding the carotenoid molecules in the hydrocarbon molecules, thereby obtaining said particles.

19. The method according to claim 18 wherein each hydrocarbon molecule is a lipid.

20-21. (canceled)

22. The method according to claim 18, wherein the ratio of carotenoid:lipid is from 1:100 to 1:1,000,000.

23. The method according to claim 18, further comprising transferring the particles or complex to a hydrocarbon-containing matrix or to a hydrocarbon-free matrix.

24. The method according to claim 18, wherein said disrupting comprises one or more of disrupting the configuration of the hydrocarbon, changing the structural organization of the hydrocarbon and packing the hydrocarbon.

25-28. (canceled)

29. The method of claim 18, wherein providing hydrocarbon molecules comprises providing cell culture, and wherein said embedding the carotenoid molecules in the hydrocarbon molecules comprises culturing the cell culture with the carotenoid, thereby stimulating cellular lipid droplet formation.

30. The method of claim 29, wherein stimulating cellular lipid droplet formation comprises at least one of stimulating mitochondria respiration, stimulating oxidative phosphorylation and growth, increasing intracellular aerobiosis, suppressing intracellular facultative or obligate anaerobic infection, stimulating formation of beige adipocytes, stimulating formation of brown adipocytes, stimulating thermogenesis, reducing glycolysis, reducing tissue hypoxia, preconditioning hypoxic tissues to reperfusion, reducing reperfusion oxidative damage, preconditioning hypoxic cancer cells and tissues for increased efficacy of chemotherapy and radiotherapy, preconditioning tissues to hypoxic conditions for increase efficacy of hypoxia, preconditioning tissues to hypoxic conditions for increase efficacy of endurance exercise therapy, preconditioning tissues to hypoxic conditions for increase efficacy of physical performance, preventing sarcopenia, treating sarcopenia, restoring depressed production of lipid containing secretions, restoring properties of lipid containing secretions, improving the immune system, protecting of the surface of the skin against dryness, protecting mucosa lining of organs, improving microbiota profile on the skin, preventing pathology-associated changes, preventing age-associated changes, preventing stress-associated changes, preventing metabolic-associated changes, attenuating pathology-associated changes, attenuating age-associated changes, attenuating stress-associated changes, attenuating metabolic-associated changes, attenuating pathology-associated changes, reversing age-associated changes, reversing stress-associated changes and reversing metabolic-associated changes.

31. The method according to claim 18, wherein said embedding comprises forming a thermodynamically favorable complex between the carotenoid molecules and the hydrocarbon molecules, that induces increase in enthalpy and/or entropy by at least 1%.

32. The particle or complex according to claim 1, wherein one or more of the following properties are altered compared to a control hydrocarbon that does not include a carotenoid:reduction of viscosity, reduction of density, reduction in chocolate bloom, increase in spreadability of fat; decreased rate of hydrocarbon itself, and/or pharmaceutical, nutraceutical or other bioactive molecules blended/incorporated into this hydrocarbon, for example lipids, by acidic oxidation/modification or degradation, improved bioavailability and bio-efficacy; decreased rate of digestibility and/or absorption of the hydrocarbon itself, and/or pharmaceutical, nutraceutical or other bioactive molecules blended/incorporated into this hydrocarbon, increased permeability and fluidity, increase in greasing and lubrication properties, reduction of freezing and melting time, use as an antifreeze itself or increased anti-freezing properties, increase in hydrophobicity and molecular gas, for example O₂, carrying/storing capacity, increase in thermal conductivity, heat dissipation, thermal energy/heat capacity and storage, accelerated heating and cooling time, reducing of cooking time and preserving thermo-sensitive vitamins and essential nutrients, increase of burning time and heat generation without increased, or with reduced, fuel consumption.
